(19) United States
(12) Patent Application Publication
(10) Pub. No.: US 2026/0284235 A1
(43) Pub. Date: Sep. 24, 2026

(54) FAP-ACTIVATED RADIOTHERANOSTICS AND USES RELATED THERETO

(71) Applicant: Trustees of Tufts College, Medford, MA (US)

(72) Inventors: William W. Bachovchin, Cambridge, MA (US); Hung-Sen Lai, Andover, MA (US); Wengen Wu, Winchester, MA (US)

(21) Appl. No.: 19/548,503

(22) Filed: Feb. 24, 2026

Related U.S. Application Data
(63) Continuation of application No. 17/554,823, filed on Dec. 17, 2021, now abandoned.
(60) Provisional application No. 63/126,617, filed on Dec. 17, 2020.

(51) Int. Cl. A61K 51/04

(52) U.S. Cl. CPC A61K 51/0482

(57) ABSTRACT

The present disclosure relates to fibroblast-activation protein (FAP)-activated theranostic prodrugs, pharmaceutical compositions comprising them, and methods of treating a disorder characterized by FAP upregulation, such as cancer.

### CROSS-REFERENCE TO RELATED APPLICATIONS

This application is a continuation of U.S. application Ser. No. 17/554,823, filed Dec. 17, 2021, which claims the benefit of priority to U.S. Provisional Patent Application No. 63/126,617, filed on Dec. 17, 2020; which is hereby incorporated by reference in its entirety.

### BACKGROUND

Fibroblast-activation protein a (FAP), also known as Seprase, is a type II integral membrane serine peptidase. FAP belongs to the dipeptidyl peptidase IV family. It is a 170 kDa homodimer containing two N-glycosylated subunits with a large C-terminal extracellular domain, in which the enzyme's catalytic domain is located. FAP, in its glycosylated form, has both post-prolyl dipeptidyl peptidase and gelatinase activities. Homologues of human FAP were found in several species, including mice and cynomolgus monkeys.

FAP is expressed selectively in reactive stromal fibroblasts of more than 90% of epithelial malignancies (primary and metastatic) examined, including lung, colorectal, bladder, ovarian and breast carcinomas, and in malignant mesenchymal cells of bone and soft tissue sarcomas, while it is generally absent from normal adult tissues (Brennen et al., Mol. Cancer Ther. 11 (2): 257-266 (2012); Garin-Chesa et al., Proc Natl Acad Sci USA 87, 7235-7239 (1990); Rettig et al., Cancer Res. 53:3327-3335 (1993); Rettig et al., Proc Natl Acad Sci USA 85, 3110-3114 (1988)). FAP is also expressed on certain malignant tumor cells.

Due to its expression in many common cancers and its restricted expression in normal tissues, FAP has been considered a promising antigenic target for imaging, diagnosis and therapy of a variety of cancers. Various approaches have been devised to exploit the selective expression of FAP in tumor stroma for clinical benefit, including monoclonal antibodies against FAP, small-molecule inhibitors of FAP enzymatic activity, FAP-activated prodrugs of cytotoxic compounds and FAP-specific CAR-T cells.

### SUMMARY

FAP-activated radiotheranostics are disclosed that will enable the selective delivery of radiodiagnostics and radiotherapeutics selectively to the tumor microenvironment. This includes radiotherapeutics designed to target other molecules or receptors in the tumor microenvironment, such as prostate specific membrane antigen, folate receptors, and somatostatin. The FAP-activation will enable the mitigation of adverse side effects by reducing exposure to normal cells and tissues that express or contain significant levels of the primary receptor or molecule being targeted, and therefore improve the therapeutic window and efficacy.

One aspect of the invention relates to FAP-activated theranostic prodrugs, and compositions comprising them. Another aspect of the invention is a method of treating a disorder characterized by fibroblast activation protein (FAP) upregulation using the prodrugs and compositions comprising them.

In certain embodiments, the subject FAP-activated theranostic prodrug agents can be represented in the general formula I:

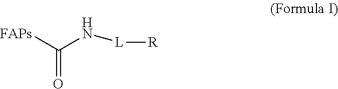

or a pharmaceutically acceptable salt thereof, wherein:
   “FAPs” represents a moiety that includes an FAPα substrate (“FAP substrate moeity”) which is cleaved by FAPα to release FAPs-C(═O)OH and NH₂-L-R;
   L is a bond, or after cleavage by FAP to release NH₂-L-R, is a self-eliminating linker; and
   R represents a ligand-targeted theranostic moiety, including a ligand for binding to a cellular target and one or more of a radioactive moiety and/or a chelating agent for chelating a radioactive moiety.

In some embodiments, enzymatic cleavage of the prodrug by fibrolast activation protein (FAP) leads to the release of the ligand-targeted theranostic moiety either as an activated ligand-targeted theranostic moiety (i.e., its pharmacologically active form) or in a form that is readily metabolized to its active form; and when released from the prodrug by FAP cleavage, the activated ligand-targeted theranostic moiety binds to the cellular target with a Kd for binding to the cellular target that is less (i.e., has a higher affinity for the cellular target) than the Kd for the prodrug binding to the cellular target.

In some embodiments, the FAP-activated theranostic prodrug is represented by Formula II:

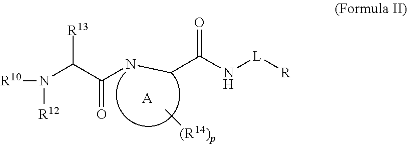

or a pharmaceutically acceptable salt thereof, wherein:
   R represents a ligand-targeted theranostic moiety, including a ligand for binding to a cellular target and one or more of a radioactive moiety and/or a chelating agent for chelating a radioactive moiety;
   A represents a 5 to 8 membered heterocycle ring;
   X is O or S;
   R¹⁰ is an amino terminal blocking group
   R¹² is hydrogen or (C₁-C₆)alkyl;
   R¹³ is hydrogen, a (C₁-C₆)alkyl (which may be straight or branched chain) or a (C₁-C₆);
   R¹⁴ is, independently for each occurrence, —(C₁-C₆)alkyl, —OH, —NH₂, or halogen;
   p is an integer from 0-6; and
   L is a bond, or after cleavage by FAP to release NH₂-L-R, is a self-eliminating linker;
and wherein
   enzymatic cleavage of the prodrug by fibrolast activation protein (FAP) leads to the release of the ligand-targeted theranostic moiety either as an activated ligand-targeted theranostic moiety (i.e., its pharmacologically active form) or in a form that is readily metabolized to its active form; and
   when released from the prodrug by FAP cleavage, the activated ligand-targeted theranostic moiety binds to the cellular target with a Kd for binding to the cellular target that is less (i.e., has a higher affinity for the cellular target) than the Kd for the
   prodrug binding to the cellular target.
      In certain preferred embodiments of Formula II, R¹² is H.

In certain embodiments of the structure of Formula II, the R¹⁰ forms an amide OR thioamide with the nitrogen to which it is attached, and the prodrug is represented by Formula IIa:

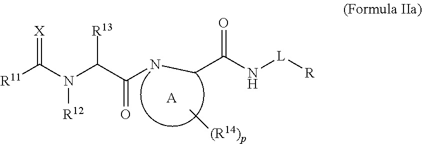

or a pharmaceutically acceptable salt thereof, wherein:
   X is O or S;
   R¹¹—(C═X) taken together represents an acyl N-terminal blocking group; or
   R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, -aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl,
wherein R¹¹ is optionally substituted with one or more substituents independently selected from the group consisting of halo, hydroxy, alkoxy, carboxy, cyano, amino, nitro, and thio; or
   R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁-C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
   AA is, independently for each occurrence, an amino acid residue;
   n is integer from 1 to 5, and
   R, A, R¹² R¹³ R¹⁴ p and L are as set forth for Formula II.

In certain preferred embodiments of Formula IIa: X is O; and/or R¹² is H. In certain preferred embodiments X is O and R¹² is H.

In certain aspects, preferred FAP-activated theranostic prodrugs represented by Formula III:

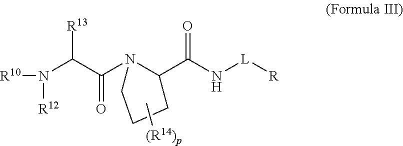

or a pharmaceutically acceptable salt thereof, wherein, R, R¹⁰, R¹², R¹³, R¹⁴, L, X and p are as defined for Formula II above.

In certain embodiments of the structure of Formula III, R¹⁰ forms an amide OR thioamide with the nitrogen to which it is attached, and the prodrug is represented in the formula IIIa

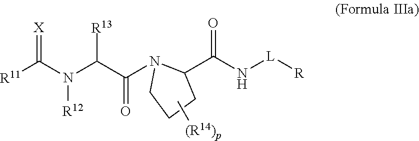

or a pharmaceutically acceptable salt thereof, wherein:
   X is O or S;
   R¹¹—(C═X) taken together represents an acyl N-terminal blocking group; or
   R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, -aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl,
   wherein R¹¹ is optionally substituted with one or more substituents independently selected from the group consisting of halo, hydroxy, alkoxy, carboxy, cyano, amino, nitro, and thio, or
   R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁-C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
   AA is, independently for each occurrence, an amino acid residue;
   n is integer from 1 to 5, and
   R, R¹² R¹³ R¹⁴ p and L are as set forth for Formula II.

In certain embodiments, the FAP-activated theranostic prodrug is represented by Formula IV:

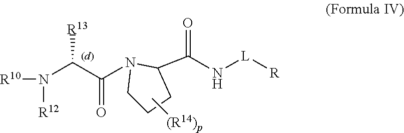

or a pharmaceutically acceptable salt thereof, wherein, R¹³ is a (C₁-C₆)alkyl (which may be straight or branched chain) or a (C₁-C₆), and R, R¹⁰, R¹², R¹³, R¹⁴, L, X and p are as defined for Formula II above.

In certain embodiments of the structure of Formula IV, R¹⁰ forms an amide OR thioamide with the nitrogen to which it is attached, and the prodrug is represented by formula Iva:

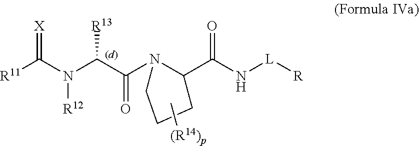

or a pharmaceutically acceptable salt thereof, wherein:
   X is O or S;
   R¹¹—(C═X) taken together represents an acyl N-terminal blocking group; or
   R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, -aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl,
   wherein R¹¹ is optionally substituted with one or more substituents independently selected from the group consisting of halo, hydroxy, alkoxy, carboxy, cyano, amino, nitro, and thio, or
   R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁-C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
   AA is, independently for each occurrence, an amino acid residue;
   n is integer from 1 to 5, and
   R, R¹² R¹³ R¹⁴ p and L are as set forth for Formula II.

In certain embodiments, R¹³ is a C₁-C₆ alkyl, such as methyl. In other embodiments, R¹³ is hydrogen.

In certain embodiments, R¹² is H.

In certain embodiments, p is 1 or 2, and R¹⁴ for each occurrence is halo. In other embodiments, p is 0.

In some embodiments, the FAP-activated radiopharmaceutical is represented by formula V:

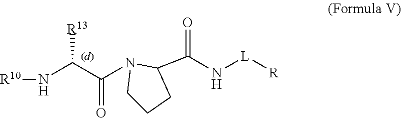

or a pharmaceutically acceptable salt thereof, or

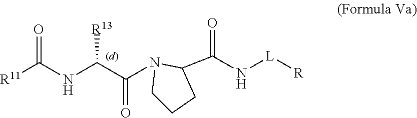

or a pharmaceutically acceptable salt thereof, wherein,
   R, R¹⁰, R¹¹, R¹³ and L are as set forth for Formula II, and

In certain embodiments, the FAP-activated theranostic prodrug is represented by formula VI:

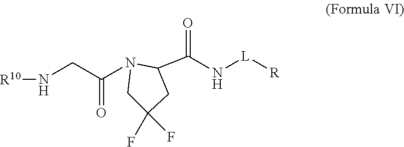

or a pharmaceutically acceptable salt thereof, or

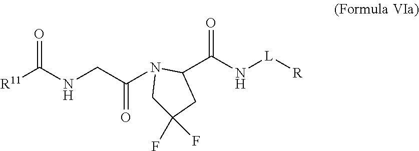

or a pharmaceutically acceptable salt thereof, wherein,
   R, R¹⁰, R¹¹ and L are as set forth for Formula II

In still other embodiments, the FAP-activated theranostic prodrug is represented by formula VII:

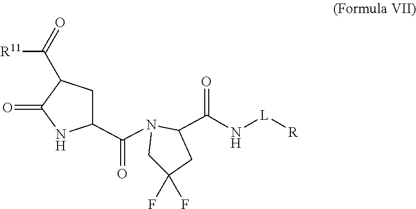

or a pharmaceutically acceptable salt thereof, wherein, R, and L are as defined for Formula II above, and —C(═O)R¹¹ forms an acyl group.

In some embodiments, X is O.

In some embodiments, R¹¹ is —(C₁-C₁₀)alkyl, —(C₁-C₁₀)alkoxy, —(C₃-C₈)cycloalkyl, —(C₆-C₁₄)aryl, aryl(C₁-C₁₀)alkyl, or 5-10-membered heteroaryl.

In some embodiments, wR¹¹ is

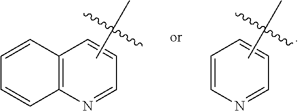

In some embodiments, n equals 1, and AA is a serine residue. In other embodiments,
n is 1 or 2.

In some embodiments, R¹¹ is (C₁-C₁₀)alkyl, (C₁-C₁₀)alkoxy, (C₁-C₁₀)alkyl-C(O)—(C₁-C₁₀)alkyl, (C₃-C₈)cycloalkyl, (C₃-C₈)cycloalkyl(C₁-C₁₀)alkyl, (C₆-C₁₄)aryl, aryl(C₁-C₁₀)alkyl, 5-10-membered heteroaryl, or 5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio,
R¹² is hydrogen;
R¹³ is a (C₁-C₆)alkyl;
R¹⁴ is absent or p is 2 and R¹⁴ is a halogen for each occurrence; and
L is a bond, or —N(H)-L- is a self-eliminating linker.

In some embodiments, —C(O)—R¹¹ forms an acyl of a carboxylic acid, such as formyl, acetyl, propionyl, butryl, oxalyl, malonyl, succinyl, glutaryl, adipoyl, acryloyl, maleoyl, fumaroyl, glycoloyl, lactoyl, pyruvoyl, glyceroyl, maloyl, oxaloacetyl, benzoyl, trifluoroacetyl or methoxysuccinyl group.

In other embodiments, R¹¹ is —(CH₂)ₘ—R^{11a}, where R^{11a} is a 5-10-membered aryl or heteroaryl group, preferably a 6-membered aryl or heteroaryl group, and m is an integer from 1 to 6, preferably 1 or 2. In some embodiments, the aryl is selected from the group consisting of benzyl, naphthalenyl, phenanthrenyl, phenolyl, and anilinyl. In other embodiments, the heteroaryl is selected from the group consisting of pyrryl, furyl, thiophenyl (a/k/a thienyl), imidazolyl, oxazolyl, thiazolyl, triazolyl, pyrazolyl, pyridinyl, pyrazinyl, pyridazinyl, and pyrimidinyl.

In some embodiments, L is a bond, while in other embodiments, L is a self-eliminating linker. The self-eliminating linker may be selected from the group consisting of

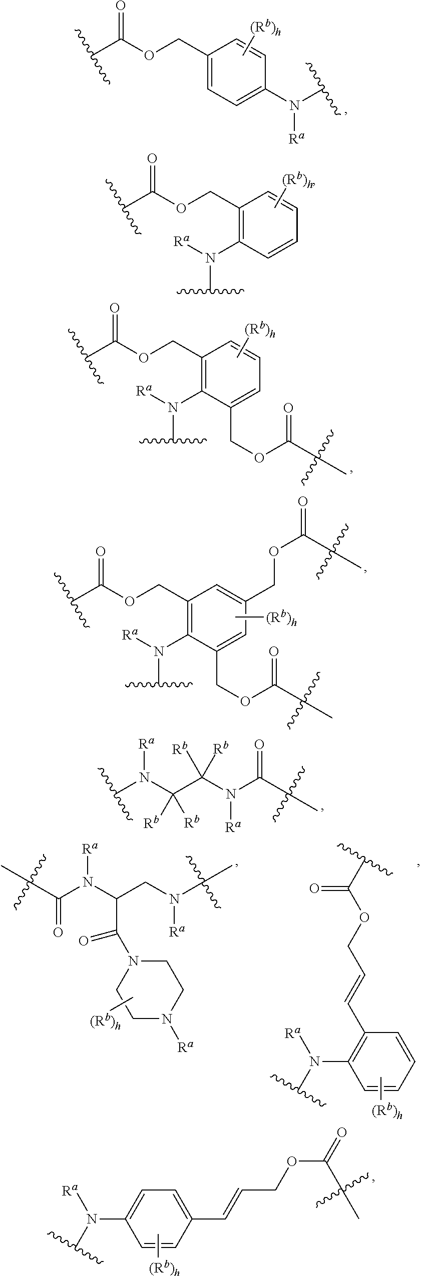

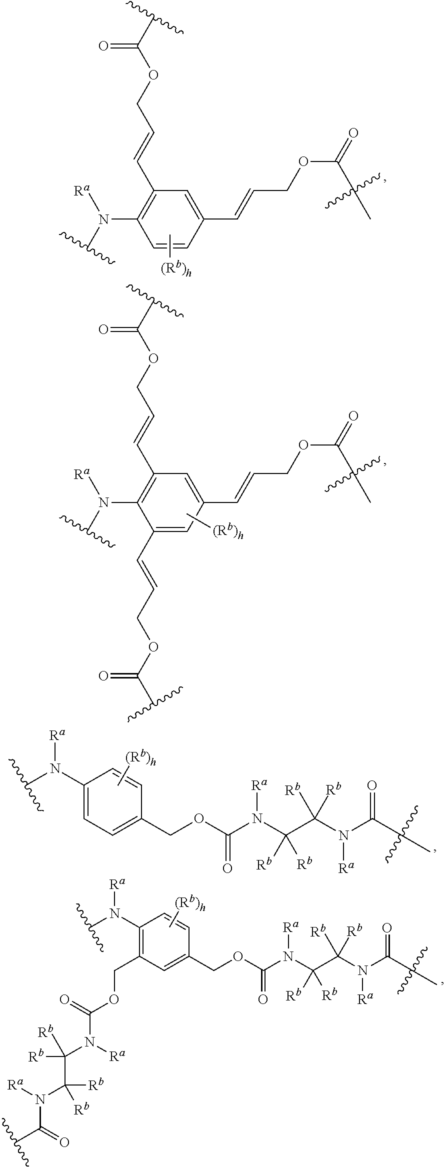

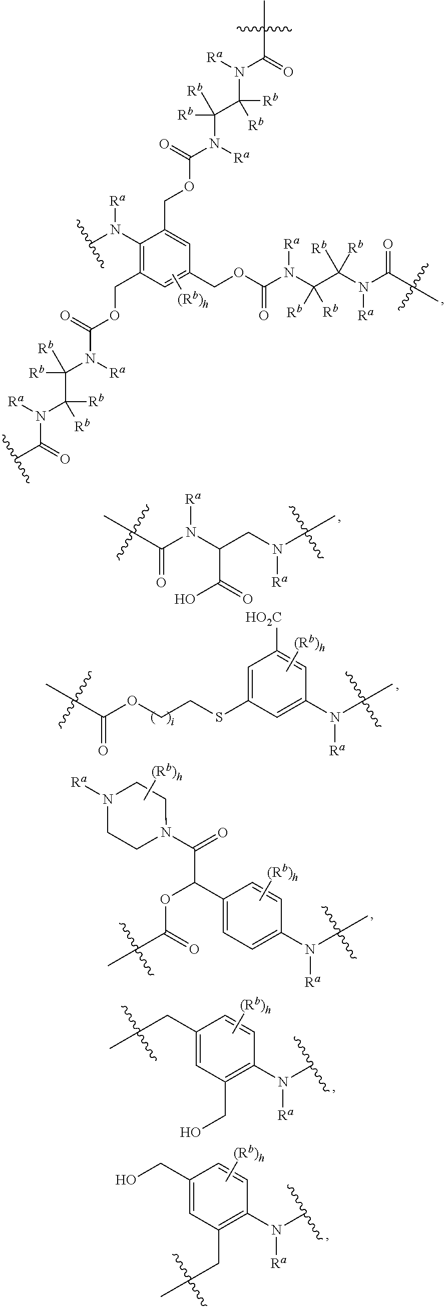

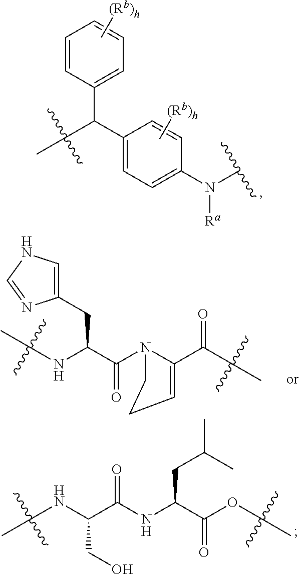

wherein
R^{a} is hydrogen, alkyl, alkenyl, alkynyl, carbocyclyl, heterocyclyl, aryl, or heteroaryl;
R^{b} is halogen, alkyl, alkenyl, alkynyl, carbocyclyl, heterocyclyl, aryl, or heteroaryl;
h is an integer from 0-8, as valency permits; and
i is an integer from 1-6.

In other embodiments, L is

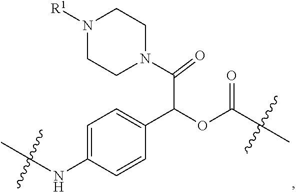

wherein R¹ is hydrogen, unsubstituted or substituted C₁₋₃ alkyl, or unsubstituted or substituted heterocyclyl.

In still other embodiments, L is selected from

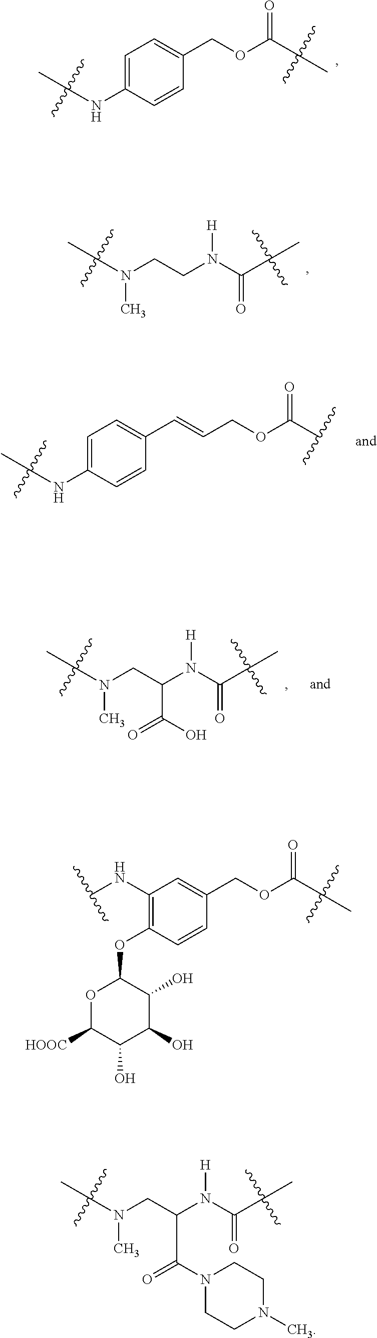

In some embodiments L is selected from

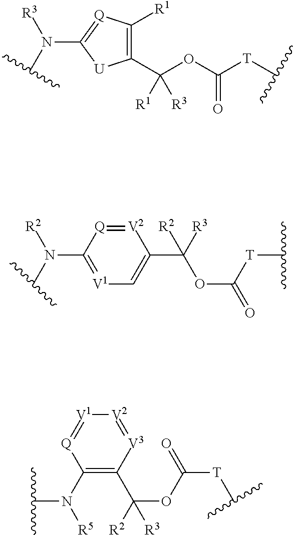

wherein
U is O, S or NR⁶;
Q is CR⁴ or N;
V¹, V² and V³ are independently CR⁴ or N provided that for formula II and III at least one of Q, V¹ and V² is N;
T is NH, NR⁶, O or S pending from said therapeutic moiety;
R¹, R², R³ and R⁴ are independently selected from H, F, Cl, Br, I, OH, —N(R⁵)₂, —N(R⁵)₃⁺, C₁-C₈ alkylhalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ halosubstituted alkyl, polyethyleneoxy, phosphonate, phosphate, C₂-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀aryl, C₆-C₂₀ substituted aryl, C₁-C₂₀ heterocycle, and C₁-C₂₀ substituted heterocycle; or when taken together, R² and R³ form a carbonyl (═O), or spiro carbocyclic ring of 3 to 7 carbon atoms; and
R⁵ and R⁶ are independently selected from H, C₁-C₈ alkyl, C₁-C₈substituted alkyl, C₂-C₈ alkenyl, C₂-C₈ substituted alkenyl, C₂-C₈alkynyl, C₂-C₈ substituted alkynyl, C₆-C₂₀aryl, C₆-C₂₀ substituted aryl, C₁-C₂₀ heterocycle, and C₁-C₂₀ substituted heterocycle;
where C₁-C₈ substituted alkyl, C₂-C₈ substituted alkenyl, C₂-C₈substituted alkynyl, C₆-C₂₀ substituted aryl, and C₂-C₂₀ substituted heterocycle are independently substituted with one or more substituents selected from F, Cl, Br, I, OH, —N(R⁵)₂, —N(R⁵)₃⁺, C₁-C₈ alkylhalide, carboxylate, sulfate, sulfamate, sulfonate C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate.

In some embodiments, L is selected from —NH—(CH₂)₄—C(═O)— or —NH—(CH₂)₃—C(═O)—, p-aminobenzyloxycarbonyl (PABC), 2,4-bis(hydroxymethyl)aniline, or benzyloxycarbonyl.

In some embodiments, the ligand-targeted theranostic moiety (R) is represented by

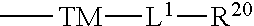

wherein:
TM represents a ligand targeting moiety that selectively binds to a cell surface feature on a target cell;
L¹ represents a bond or a linker; and
R²⁰ represents a radioactive moiety, a chelating agent, a fluorescent moeity, a photoacoustic reporting molecule, a Raman-active reporting molecule, a contrast agent, or a detectable nanoparticle.

In some embodiments, the ligand targeting moiety is a folate receptor ligand, preferably folic acid or folic acid analogs, preferably etarfolatide, vintafolide, leucovorin and methotrexate.

In some embodiments, the ligand targeting moiety is a somatostatin or a somatostatin analogs, preferably octreotate, octreotide pentetreotide, lanreotide, vapreotide, pasireotide, seglitide, benereotide, KE-108, SDZ-222-100, Sst3-ODN-8, CYN-154806, JR11, J2156, SRA-880, ACQ090, P829, SSTp-58, SSTp-86, BASS or somatoprim.

In still other embodiments, the ligand targeting moiety is an αIIbβ3-targeted ligand, such as RGD or an RGD analog, preferably cyclo(-Arg-Gly-Asp-D-Phe Val-) [“c(RGDfV)” ], c(RGDfK), c(RGDfC), c(RADfC), c(RADfK), c(RGDfE), c(RADfE), RGDSK, RADSK, RGDS, c(RGDyC), c(RADyC), c(RGDyE), c(RGDyK), c(RADyK), H-E[c(RGDyK)]₂, EMD 12194, DMP728, DMP757 and SK&F107260.

In some embodiments, the targeted theranostic moiety (R) is

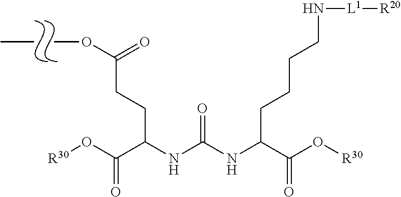

wherein
R³⁰ represents, independently for each occurrence, a hydrogen or a lower alkyl.

In some embodiments, -L¹-R²⁰ is represented by

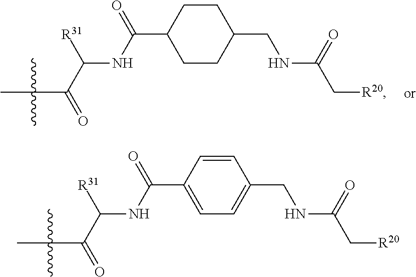

wherein and R³¹ is —(CH₂)ₚ-aryl or is —(CH₂)ₚ-heteroaryl, and p is 0, 1, 2, 3 or 4.

In some embodiments, R³¹ is —CH₂-aryl, where the aryl group is a C₆ to C₁₂ aryl, and is a monocyclic or bicyclic fused ring, preferably napthalene, for example, -L¹-R²⁰ can be represented by

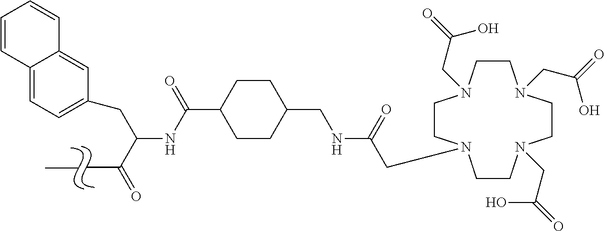

In some embodiments, R²⁰ is a F¹⁸ containing moiety, for example, -L-R²⁰ can be selected from

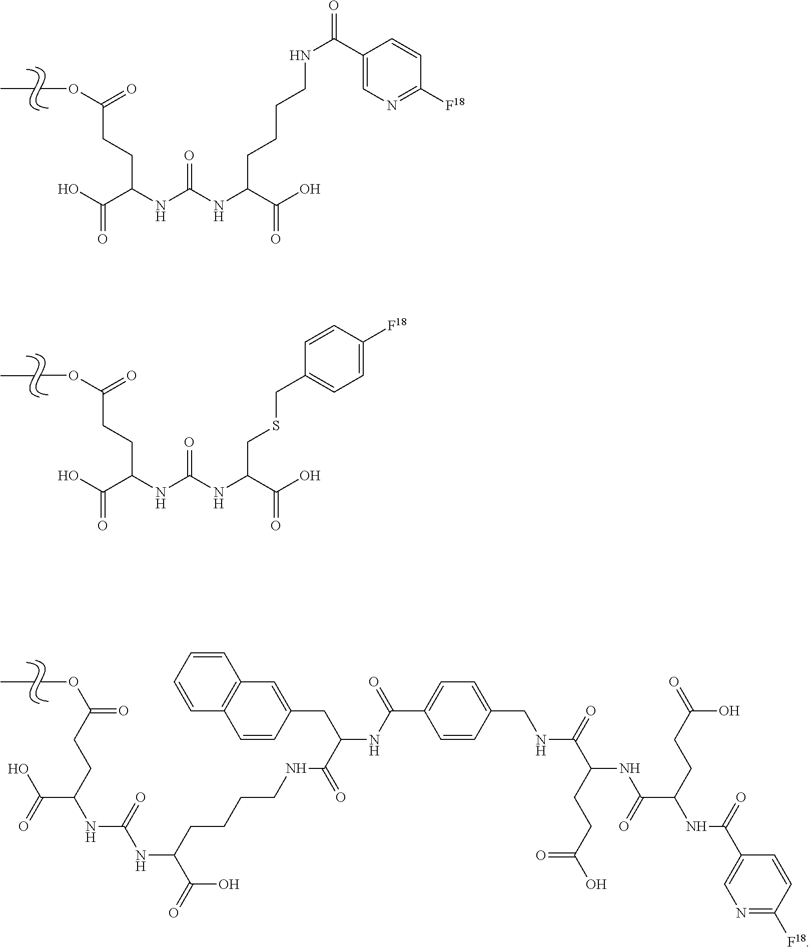

In some embodiments, the ligand-targeted theranostic moiety (R) includes folic acid or a folic acid analog chosen from

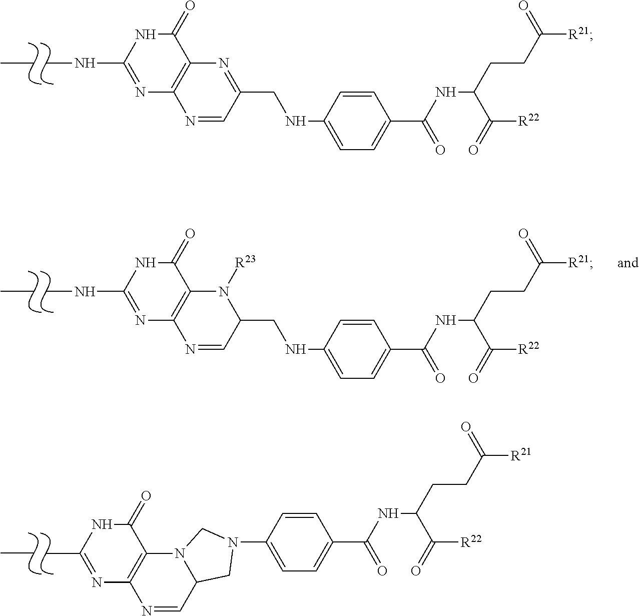

wherein
R²¹ represents H, and R²² represents —NH—(CH₂)_{q}—R²⁰, —NH—(CH₂)_{q}—NH—C(O)—(CH₂)_{q}—R²⁰ or —NH—(CH₂)_{q}—C(O)—(CH₂)_{q}—R²⁰; or
R²² represents H, and R²¹ represents —NH—(CH₂)_{q}—R²⁰ or —NH—(CH₂)_{q}—C(O)—(CH₂)_{q}—R²⁰ or
one of R²¹ or R²² represents H, and the other is selected from the group

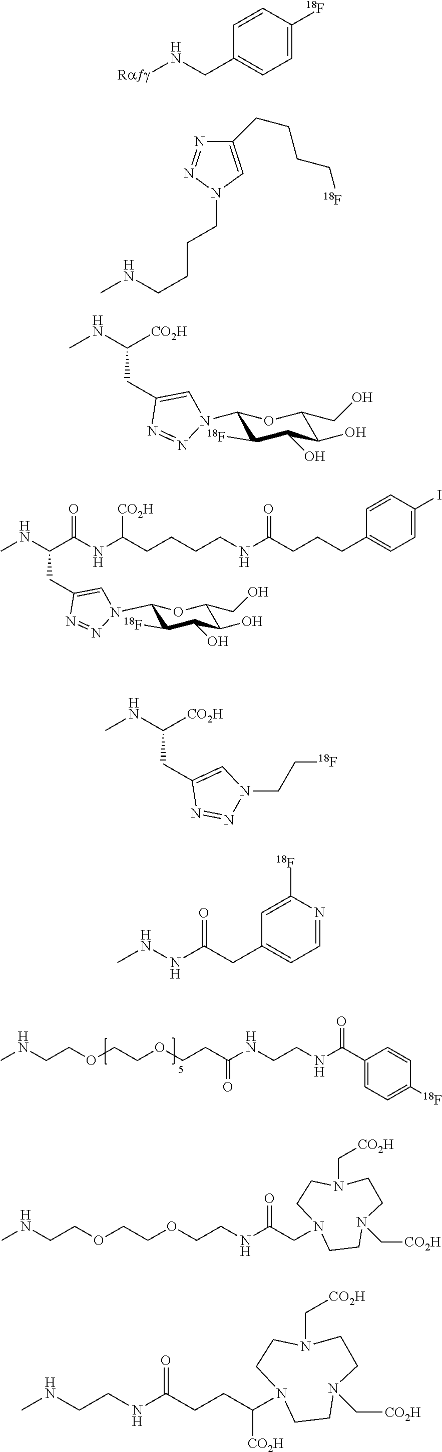

R²³ represents H, —CH₃, —CH₂CH₃, or —CO₂H;
and
q, independently for each occurrence, is 0, 1, 2, 3 or 4.

In other embodiments, R²¹ represents —NH—CH₂—R²⁰, —NH—CH₂—C(O)—R²⁰, —NH—C(O)—CH₂—R²⁰, —NH—CH₂—C(O)—CH₂—R²⁰ or —NH—(CH₂)₂—NH—C(O)—CH₂—R²⁰ and R²² represents H.

In still other embodiments, R²¹ represents H, and R²² represents —NH—CH₂—R²⁰, —NH—CH₂—C(O)—R²⁰, —NH—C(O)—CH₂—R²⁰, —NH—CH₂—C(O)—CH₂—R²⁰, or —NH—(CH₂)₂—NH—C(O)—CH₂—R²⁰.

In some embodiments, the ligand-target theranostic moiety (R) is

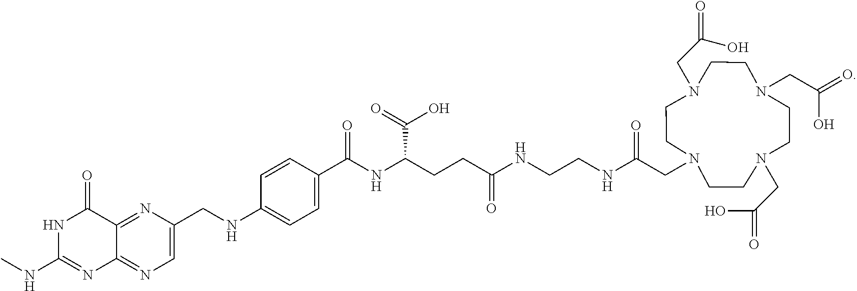 [SMILES: CNc1nc2ncc(CNc3ccc(C(=O)N[C@@H](CCC(=O)NCCNC(=O)CN4CCN(CC(=O)O)CCN(CC(=O)O)CCN(CC(=O)O)CC4)C(=O)O)cc3)nc2c(=O)[nH]1]

In still other embodiments, the ligand-targeted theranostic moiety (R) includes folic acid or a folic acid analog labeled with a radioistope chosen from

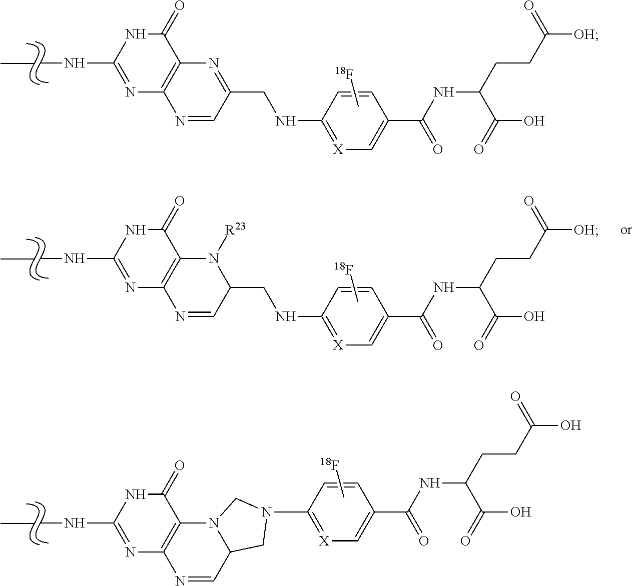

wherein R²³ represents H, —CH₃, —CH₂CH₃, or —CO₂H and X represents CR⁴⁰ or N, wherein R⁴⁰ is H or lower alkyl.

In some embodiments, R is chosen from

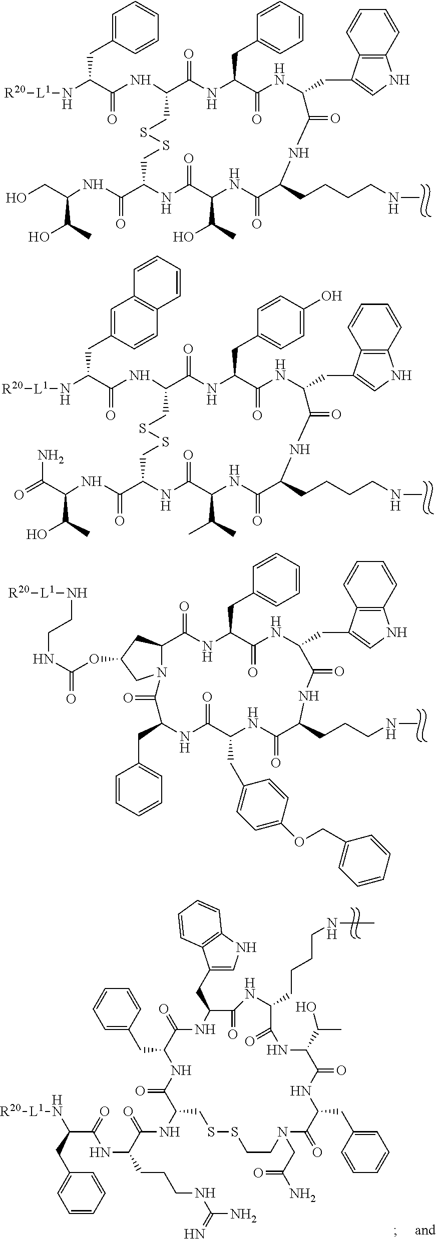

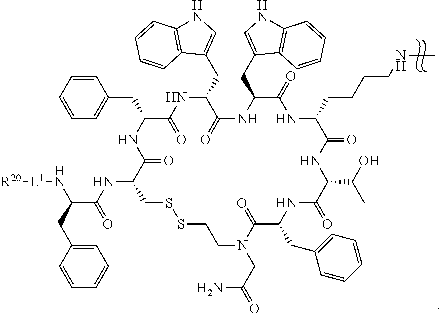

In some embodiments, R²⁰ is chosen from

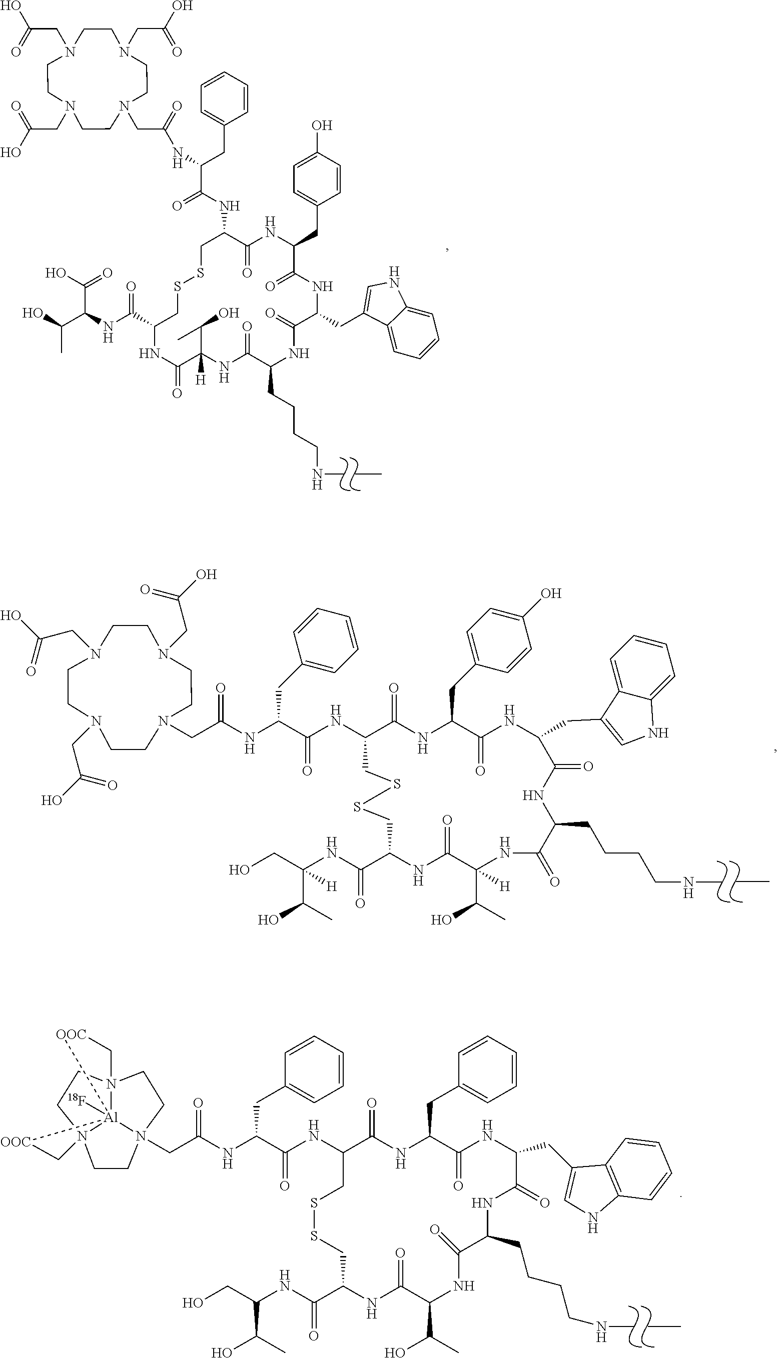

In still other embodiments, R²⁰-L¹- is

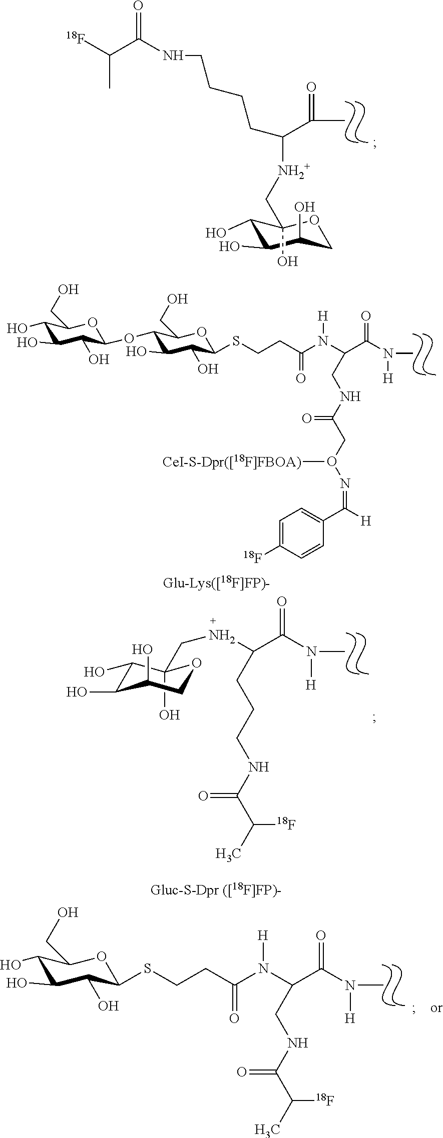

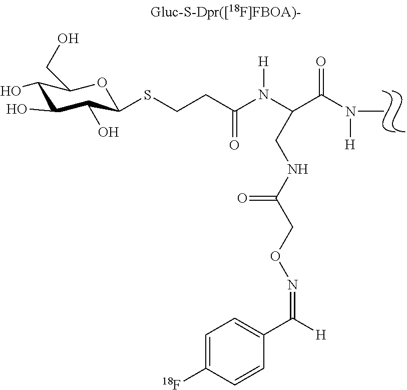

In some embodiments, R is a ligand for an extracellular receptor. For example, R is a ligand for an extracellular receptor that undergoes intracellular internalization and can transport R, when released from the prodrug, into one or more intracellular compartments of cells that express the extracellular receptor. In still other embodiments, the cellular target is expressed by cells in a tissue in which FAP expression is upregulated. In further embodiments, the tissue in which FAP expression is upregulated is a tumor.

In some embodiments, R is an analog, such as a peptide analog, that binds to a peptide hormone receptor. For example, R can be a peptide analog, of somatostatin, bombesin, calcitonin, oxytocin, EGF, α-melanocyte-stimulating hormone, minigastrin, neurotesin or neuropeptide Y (NPY).

In some embodiments, R is ligand that binds to integrin αvβ3, a gastrin-releasing peptide receptor (GRPR), a somatostatin receptor (such as somatostatin receptor subtype 2), a melanocortin receptor, a cholecystokinin-2 receptor, a neuropeptide Y receptor or a neurotensin receptor.

In some embodiments, R is a ligand that binds to a type II membrane protein, such as a prostate-specific membrane antigen (PSMA). For example, the theranostic prodrug may have a structure selected from:

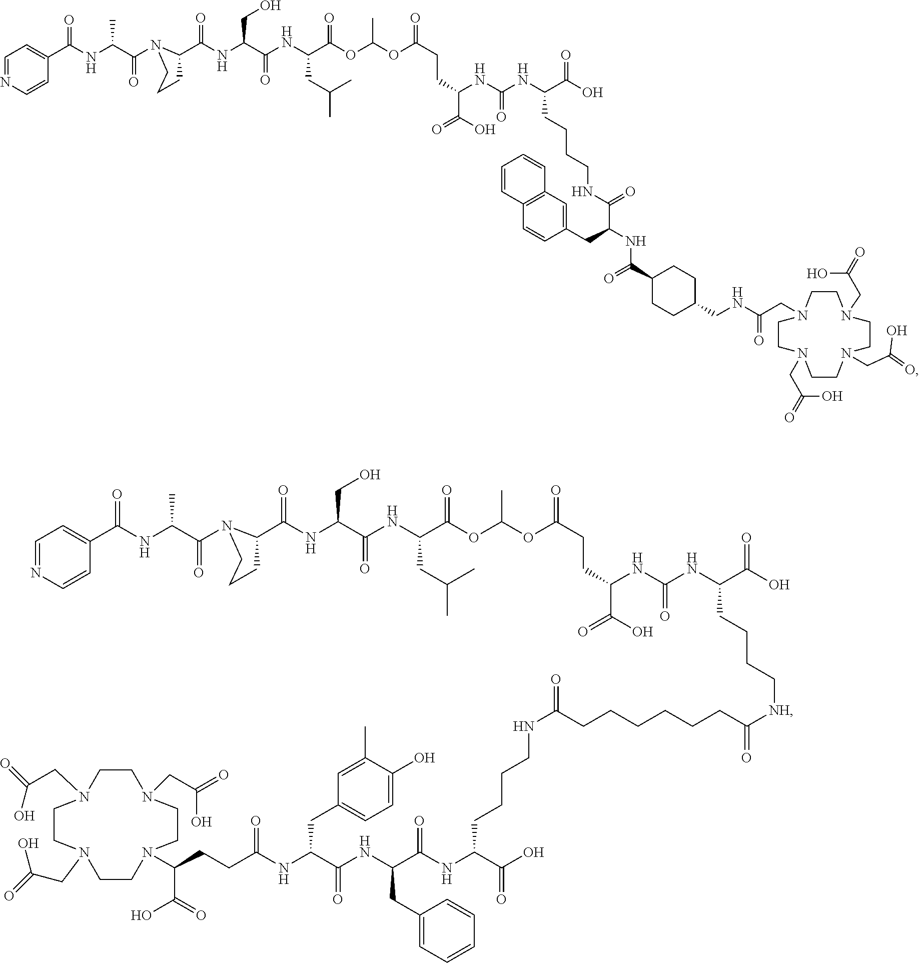

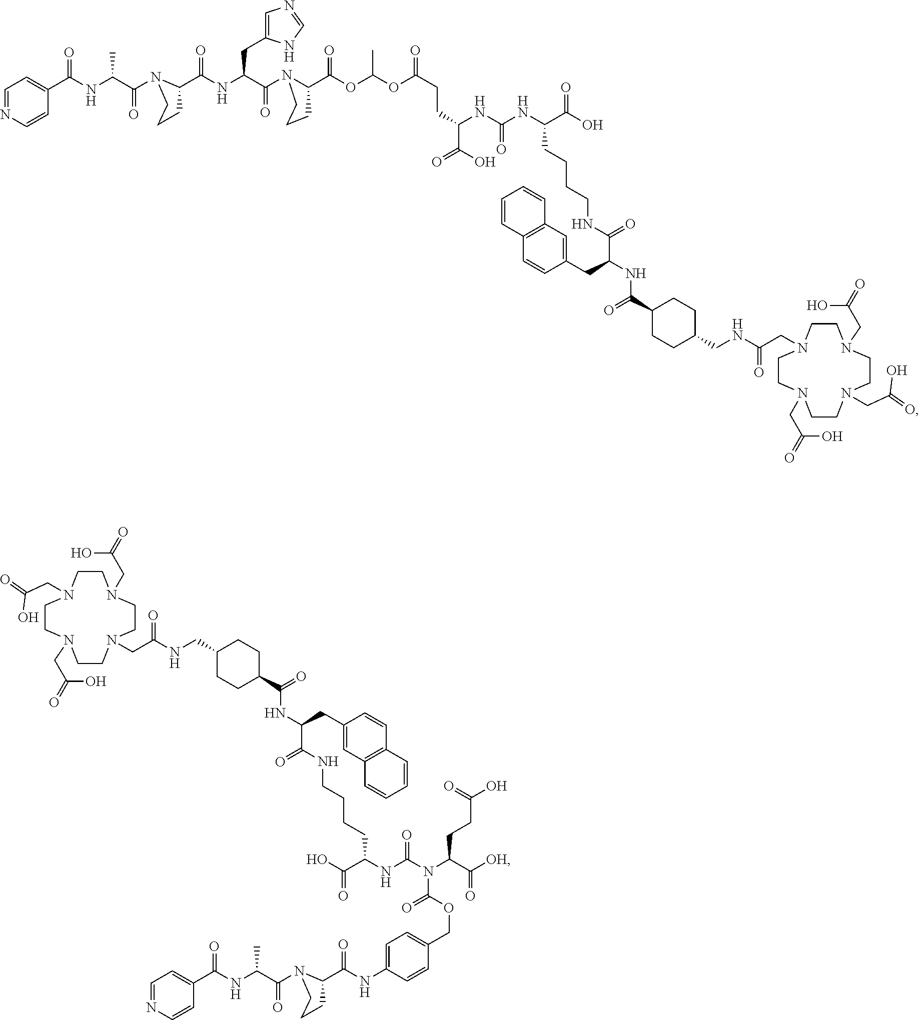

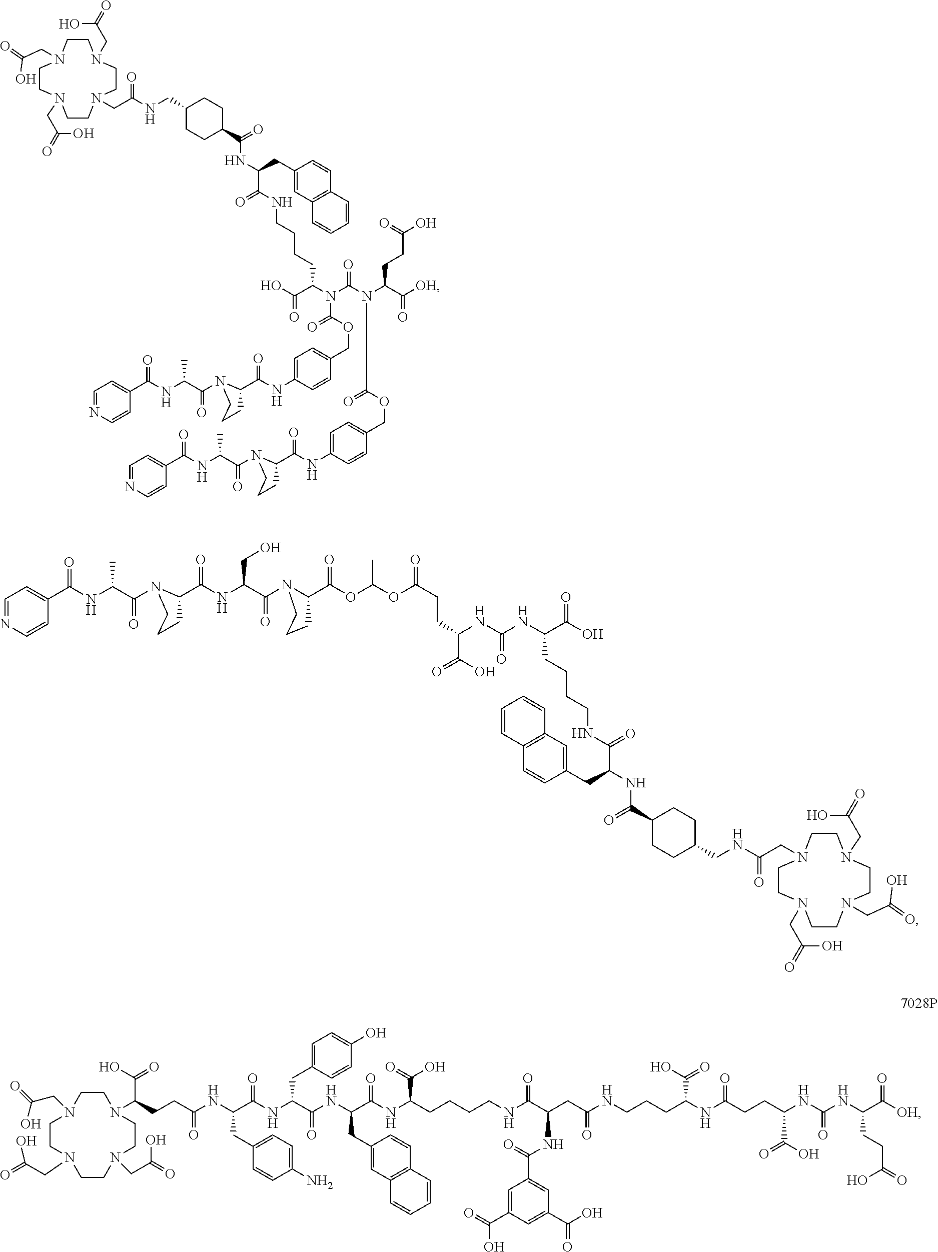

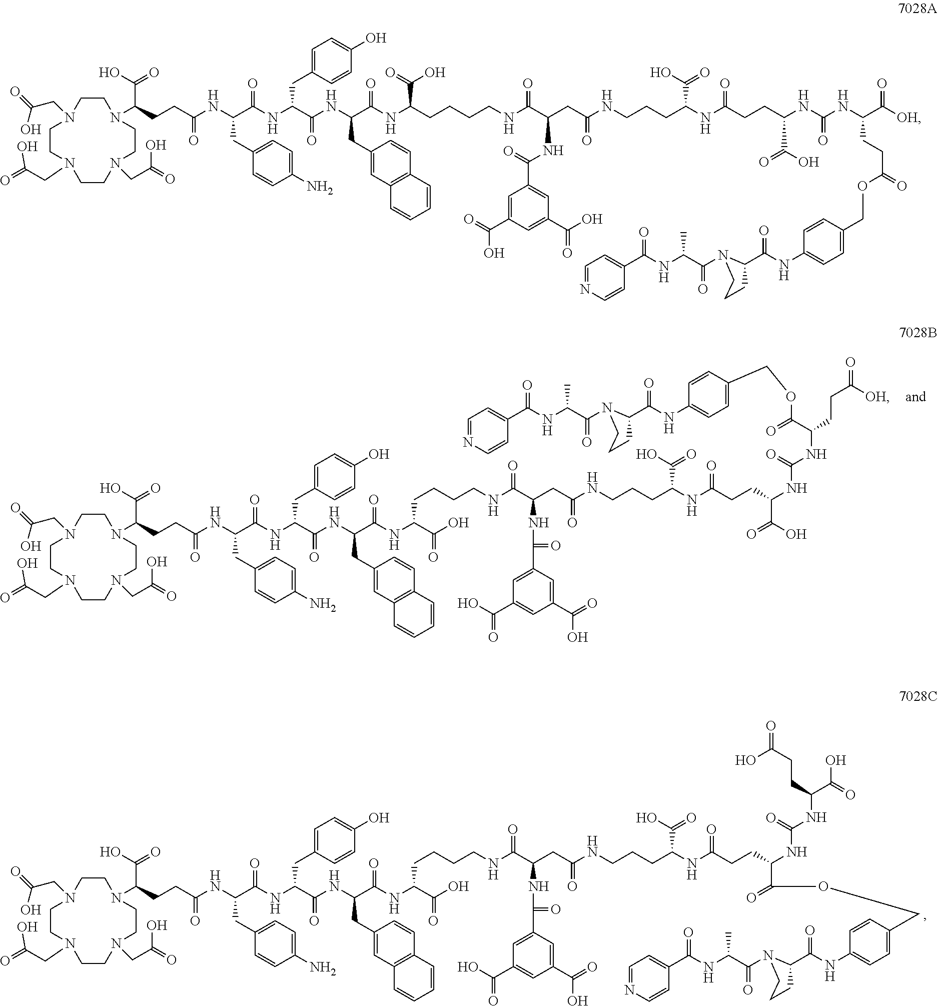

or a pharmaceutically acceptable salt thereof,
which may optionally include a radioisotope chelated thereto.
   In some embodiments, R is a ligand that binds to a somatostatin receptor, such as:

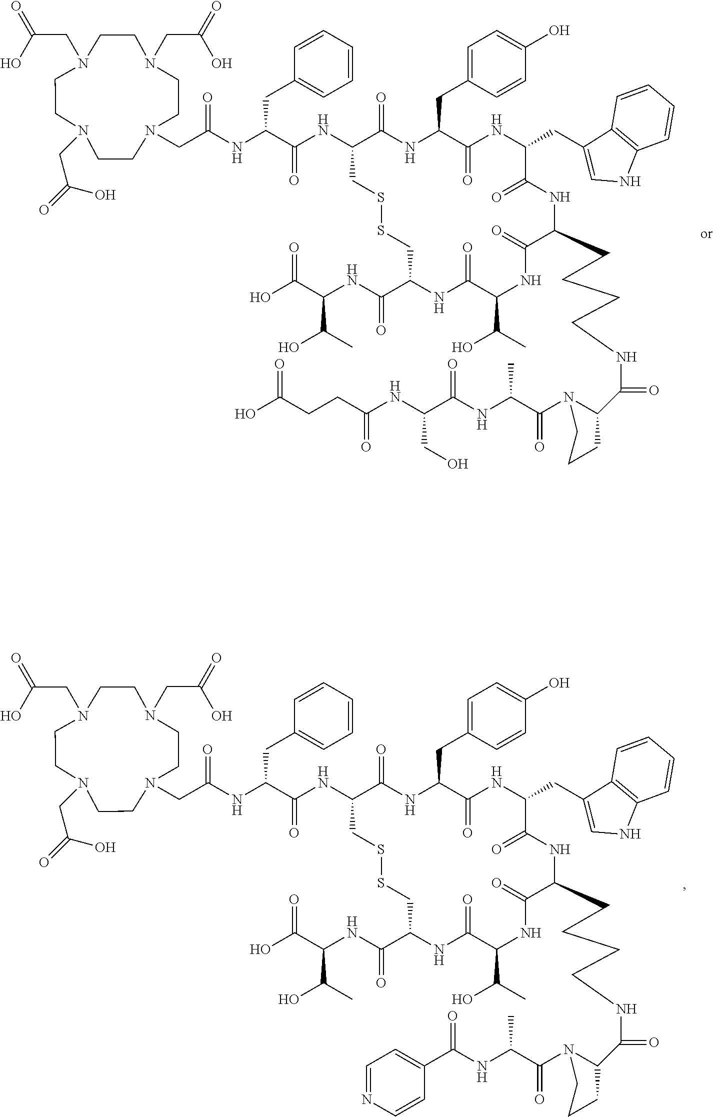

or a pharmaceutically acceptable salt thereof,
which may optionally include a radioisotope chelated thereto.

In some embodiments, the ligand includes a chelating agent which is, or is capable of, chelating a radioactive metal or semi-metal isotopes, such as ¹⁸F, ⁴³K, ⁴⁷Sc, ⁵¹Cr, ⁵⁷Co, ⁵⁸Co, ⁵⁹Fe, ⁶⁴Cu, ⁶⁷Cu, ⁶⁷Ga, ⁶⁸Ga, ⁷¹Ge, ⁷²As, ⁷²Se, ⁷⁵Br, ⁷⁶Br, ⁷⁷As, ⁷⁷Br, ⁸¹Rb, ⁸⁸Y, ⁹⁰Y, ⁹⁷Ru, ^{99m}Tc, ¹⁰⁰Pd, ^{101m}Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, ¹²³I, ¹²⁴I, ¹²⁵I, ¹²⁷Cs, ¹²⁸Ba, ¹²⁹Cs, ¹³¹Cs, ¹³¹I, ¹³⁹La, ¹⁴⁰La, ¹⁴²Pr, ¹⁴³Pr, ¹⁴⁹Pm, ¹⁵¹Eu, ¹⁵³Eu, ¹⁵³Sm, ¹⁵⁹Gr, ¹⁶¹Tb, ¹⁶⁵Dy, ¹⁶⁶Ho, ¹⁶⁹Eu, ¹⁷⁵Yb, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁸Au, ¹⁹⁹Ag, ¹⁹⁹Au, ²⁰¹Tl, ²⁰³Pb, ²¹¹At, ²¹²Bi, ²¹²Pb, ²¹³Bi, ²²⁵Ac and ²²⁷Th.

In some embodiments, the prodrug has a k_{cat}/Kₘ for cleavage by FAP at least 10-fold greater than for cleavage by prolyl endopeptidase.

The present disclosure also provides a pharmaceutical composition, comprising an FAP-activated theranostic prodrug of any one of the preceding claims, or a pharmaceutically acceptable salt thereof; and a pharmaceutically acceptable carrier.

The present disclosure also provides a method of treating a disorder characterized by fibroblast activation protein (FAP) upregulation, comprising administering to a subject in need thereof a therapeutically effective amount of the FAP-activated theranostic prodrug of any one of the preceding claims, or a pharmaceutically acceptable salt thereof. In some embodiments, the disorder characterized by FAP upregulation is cancer.

In further aspects, methods of treating a subject having prostate cancer are provided which may suitably comprise administering to a subject in need thereof a therapeutically effective amount of the FAP-activated theranostic prodrug of any one of the preceding claims, or a pharmaceutically acceptable salt thereof.

Other aspects of the invention are disclosed below.

### BRIEF DESCRIPTION OF DRAWINGS

FIG. 1 depicts a synthetic scheme for compound 7885.

FIG. 2 depicts a synthetic scheme for compound 6885.

FIG. 3 depicts a synthetic scheme for compound 6879.

FIG. 4 depicts a synthetic scheme for compound 6880.

FIG. 5 depicts a synthetic scheme for compound 6886.

FIG. 6 is a graph depicting the results of an assay of PSMA activity for compounds 7028P-7028A/B/C. Data are the average of three readings (standard error of the mean plitted but not visible).

FIG. 7 depicts a synthetic scheme for compound 6970B ester.

FIG. 8 depicts a synthetic scheme for compound 7014.

FIG. 9 depicts a synthetic scheme for compound 7366P5.

FIG. 10 depicts a synthetic scheme for compound 7366.

FIG. 11 is a graph depicting the results of FAP Activation of 6970B Isomer 1&2, 7366 Crude using 100 uM Substrate, 50 nM FAP

FIG. 12 depicts LC/MS spectra of 6970B Isomer 1.

FIG. 13 depicts LC/MS spectra of 6970B Isomer 2.

FIG. 14 depicts LC/MS spectra of 6970B Isomer 1&2.

FIG. 15 depicts LC/MS spectra of 7366.

### DETAILED DESCRIPTION

### I. Overview

Targeting the tumor microenvironment with an FAP-activated radiopharmaceutical prodrug is believed to have multiple modes of anti-tumor action, but principally relies on the induction of DNA damage in tumor cells by ionizing radiation emitted locally from neighboring CAFs targeted by the therapy. FAP-activated radiotherapy can deliver ionizing radiation to cancer cells and the tumor stroma. Combining α- and β-emitters may improve these dual antitumor effects via short-range α-radiation to CAFs and mid- to long-range β-radiation to cancer cells.

FAP-positive CAFs are found in more than 90% of epithelial cancers, therefore representing a potential pan-cancer prodrug activating enzyme. Targeting ligand-directed radiopharmaceuticals (and other theranostic agents) to tumors by generating FAP-activated prodrug versions is a means to deliver the activated ligand-directed radiopharmaceutical, i.e., selectively into a tumor in a form which, after cleavage by FAP in the tumor, is able to bind to the cellular target to which the native ligand would bind. The circulating prodrug form, as its binding to the cellular target is greatly reduced relative to the ligand released from the prodrug by FAP cleavage, is taken up to a lesser extent in non-tumor tissues (such as salivary glands, kidneys, etc) than the activated ligand-directed radiopharmaceutical and can result in an enhanced therapeutic index for the prodrug (relative to the activated ligand-directed radiopharmaceutical if administered in that form), better efficacy or both.

In certain embodiments, the subject FAP-activated theranostic prodrug agents can be represented in the general formula:

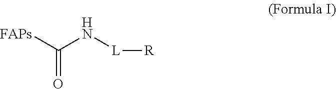

or a pharmaceutically acceptable salt thereof, wherein:
   “FAPs” represents a moiety that includes an FAPα substrate (“FAP substrate moeity”) which is cleaved by FAPα to release FAPs-C(═O)OH and NH₂-L-R;
   L is a bond, or after cleavage by FAP to release NH₂-L-R, is a self-eliminating linker; and
   R represents a ligand-targeted theranostic moiety, including a ligand for binding to a cellular target and one or more of a radioactive moiety and/or a chelating agent for chelating a radioactive moiety; and
      wherein enzymatic cleavage of the prodrug by fibrolast activation protein (FAP) leads to the release of the ligand-targeted theranostic moiety either as an activated ligand-targeted theranostic moiety (i.e., its pharmacologically active form) or in a form that is readily metabolized to its active form; and when released from the prodrug by FAP cleavage, the activated ligand-targeted theranostic moiety binds to the cellular target with a Kd for binding to the cellular target that is less (i.e., has a higher affinity for the cellular target) than the Kd for the prodrug binding to the cellular target.

In certain embodiments, the FAP substrate moiety has a kcat/Km for cleavage by FAPα at least 10-fold, at least 100-fold, 1000-fold, 5000-fold, or 10,000-fold greater than a kcat/Km for cleavage by prolyl endopeptidase (EC 3.4.21.26; PREP).

In certain embodiments, the activated ligand-targeted theranostic moiety (i.e., when released from the prodrug), has a Kd for binding the cellular target that is at least 2 times less than the Kd for the prodrug binding to the cellular target, and more preferably at least 5, 10, 20, 50, 100, 500 or even 1000 times less.

In certain embodiments, the prodrug may be further characterized by one or more of the following features:
the prodrug has a therapeutic index that is at least 2 times greater than the therapeutic index of the activated ligand-targeted theranostic moiety itself (i.e., if administered in its active form), and more preferably at least 5, 10, 50, 100, 250, 500, 1000, 5000, or even 10,000 times greater;
the activated ligand-targeted theranostic moiety is present at a higher local concentration in the target tissue, i.e., tumor or other target tissue expressing FAP, relative to the concentration of circulating activated ligand-targeted theranostic moiety, for instance at concentrations at least 2 times greater, and more preferably at least 5, 10, 100, or even 1000 times greater;
the maximum tolerated dose of the prodrug is at least 2 times greater than the maximum tolerated dose of the activated ligand-targeted theranostic moiety if administered alone in its active form, and even more preferably at least 5, 10, 100, or even 1000 times greater;
the receptor-mediated uptake of the prodrug is at least 50% less than the receptor-mediated uptake of the activated ligand-targeted theranostic moiety, and even more preferably at least 60%, 70%, 80%, 90%, 95%, 98%, 99%, or even 99.9% less; and/or
the cell permeability of the prodrug is at least 50% less than the cell permeability of the activated ligand-targeted theranostic moiety, and even more preferably at least 60%, 70%, 80%, 90%, 95%, 98%, 99%, or even 99.9% less; and/or
the prodrug has a molecular weight of less than 5000 amu;
the circulating half-life of the prodrug is at least 25% longer than the circulating half-life of the activated ligand-targeted theranostic moiety alone, and even more preferably at least 50%, 75%, 100%, 150%, 200%, 500%, 750%, or even 1000% longer.

### II. Definitions

The term “fibroblast activation protein (FAP)” as used herein is also known under the term “seprase”. Both terms can be used interchangeably herein. Fibroblast activation protein is a homodimeric integral protein with dipeptidyl peptidase IV (DPPIV)-like fold, featuring an alpha/beta-hydrolase domain and an eight-bladed beta-propeller domain.

As used herein the term “SPECT” is an abbreviation for single photon emission computed tomography.

As used herein the term “PET” is an abbreviation for positron emission tomography.

As used herein the term “CT” is an abbreviation for computed tomography.

As used herein the term “MRI” is an abbreviation for magnetic resonance imaging.

As used herein the term “SIRT” is an abbreviation for selective internal radiation therapy.

As used herein the term “EDTA” is an abbreviation for ethylenediaminetetraacetic acid.

As used herein the term “DOTA” is an abbreviation for 1,4,7,10-tetraazacyclododecane-1,4,7,10-N,N′,N″,N′″-tetraacetic acid.

As used herein the term “DTPA” is an abbreviation for diethylenetriaminepentaacetic acid.

As used herein the term metal “chelating agent” or “chelator” refers to a polydentate ligand that forms two or more separate coordinate bonds with a single central atom, in particular with a radioactive isotope.

The term “therapeutically effective amount” as used herein includes within its meaning a non-toxic but sufficient amount of a compound or composition for use in the invention to provide the desired therapeutic effect. The exact amount required will vary from subject to subject depending on factors such as the species being treated, the age, weight and general condition of the subject, co-morbidities, the severity of the condition being treated, the particular agent being administered and the mode of administration and so forth. Thus, for any given case, an appropriate “effective amount” may be determined by one of ordinary skill in the art using only routine methods.

The term “radioactive moiety” as used herein refers to a molecular assembly which carries a radioactive nuclide. The nuclide is bound either by covalent or coordinate bonds which remain stable under physiological conditions. Examples are [1311]-3-iodobenzoic acid or 68GaDOTA.

A “fluorescent isotope” as used herein emits electromagnetic radiation after excitation by electromagnetic radiation of a shorter wavelength.

A “radioisotope” as used herein is a radioactive isotope of an element (included by the term “radionuclide”) emitting α-, β- or γ-radiation.

The term “radioactive drug” is used in the context of the present invention to refer to a biologic active compound which is modified by a radioisotope. Especially intercalating substances can be used to deliver the radioactivity to direct proximity of DNA (e.g. a ¹³¹I carrying derivative of Hoechst-33258).

The term “chelating agent” or “chelate” are used interchangeably in the context of the present invention and refer to a molecule, often an organic one, and often a Lewis base, having two or more unshared electron pairs available for donation to a metal ion. The metal ion is usually coordinated by two or more electron pairs to the chelating agent. The terms, “bidentate chelating agent”, “tridentate chelating agent, and “tetradentate chelating agent” refer to chelating agents having, respectively, two, three, and four electron pairs readily available for simultaneous donation to a metal ion coordinated by the chelating agent. Usually, the electron pairs of a chelating agent forms coordinate bonds with a single metal ion; however, in certain examples, a chelating agent may form coordinate bonds with more than one metal ion, with a variety of binding modes being possible.

The term “fluorescent dye” is used in the context of the present invention to refer to a compound that emits visible or infrared light after excitation by electromagnetic radiation of a shorter and suitable wavelength. It is understood by the skilled person, that each fluorescent dye has a predetermined excitation wavelength.

The term “contrast agent” is used in the context of the present invention to refer to a compound which increases the contrast of structures or fluids in medical imaging. The enhancement is achieved by absorbing electromagnetic radiation or altering electromagnetic fields.

The term “paramagnetic” as used herein refers to paramagnetism induced by unpaired electrons in a medium. A paramagnetic substance induces a magnetic field if an external magnetic field is applied. Unlike diamagnetism the direction of the induced field is the same as the external field and unlike ferromagnetism the field is not maintained in absence of an external field.

The term “therapeutically effective amount” as used herein includes within its meaning a non-toxic but sufficient amount of a compound or composition for use in the invention to provide the desired therapeutic effect. The exact amount required will vary from subject to subject depending on factors such as the species being treated, the age, weight and general condition of the subject, co-morbidities, the severity of the condition being treated, the particular agent being administered and the mode of administration and so forth. Thus, for any given case, an appropriate “effective amount” may be determined by one of ordinary skill in the art using only routine methods.

The term “alkyl” refers to a saturated straight or branched carbon chain. Preferably, the chain comprises from 1 to 10 carbon atoms, i.e. 1, 2, 3, 4, 5, 6, 7, 8, 9 or 10 e.g. methyl, ethyl, methyl, ethyl, propyl, iso-propyl, butyl, iso-butyl, tert-butyl, pentyl, hexyl, pentyl, or octyl. Alkyl groups are optionally substituted.

The term “heteroalkyl” refers to a saturated straight or branched carbon chain. Preferably, the chain comprises from 1 to 9 carbon atoms, i.e. 1, 2, 3, 4, 5, 6, 7, 8, 9 e.g. methyl, ethyl, propyl, iso-propyl, butyl, iso-butyl, sec-butyl, tert-butyl, pentyl, hexyl, pentyl, octyl, which is interrupted one or more times, e.g. 1, 2, 3, 4, 5, with the same or different heteroatoms. Preferably the heteroatoms are selected from O, S, and N, e.g. —O—CH₃, —S—CH₃, —CH₂—O—CH₃, —CH₂—O—CH₂—CH₃, —CH₂—S—CH₃, —CH₂—S—CH₂—CH₃, —CH₂—CH₂—O—CH₃, —CH₂—CH₂—O—CH₂—CH₃, —CH₂—CH₂—S—CH₃, —CH₂—CH₂—S—CH₂—CH₃ etc. Heteroalkyl groups are optionally substituted.

The terms “cycloalkyl” and “heterocycloalkyl”, by themselves or in combination with other terms, represent, unless otherwise stated, cyclic versions of “alkyl” and “heteroalkyl”, respectively, with preferably 3, 4, 5, 6, 7, 8, 9 or 10 atoms forming a ring, e.g. cyclopropyl, cyclobutyl, cyclopentyl, cyclohexyl, cycloheptyl, cyclooctyl etc. The terms “cycloalkyl” and “heterocycloalkyl” are also meant to include bicyclic, tricyclic and polycyclic versions thereof. The term “heterocycloalkyl” preferably refers to a saturated ring having five of which at least one member is a N, O or S atom and which optionally contains one additional O or one additional N; a saturated ring having six members of which at least one member is a N, O or S atom and which optionally contains one additional O or one additional N or two additional N atoms; or a saturated bicyclic ring having nine or ten members of which at least one member is a N, O or S atom and which optionally contains one, two or three additional N atoms. “Cycloalkyl” and “heterocycloalkyl” groups are optionally substituted. Additionally, for heterocycloalkyl, a heteroatom can occupy the position at which the heterocycle is attached to the remainder of the molecule. Examples of cycloalkyl include cyclopropyl, cyclobutyl, cyclopentyl, cyclohexyl, 1-cyclohexenyl, 3-cyclohexenyl, cycloheptyl, spiro[3,3]heptyl, spiro[3,4]octyl, spiro[4,3]octyl, spiro[3,5]nonyl, spiro[5,3]nonyl, spiro[3,6]decyl, spiro[6,3]decyl, spiro[4,5]decyl, spiro[5,4]decyl, bicyclo[2.2.1]heptyl, bicyclo[2.2.2]octyl, adamantyl, and the like. Examples of heterocycloalkyl include 1-(1,2,5,6-tetrahydropyridyl), 1-piperidinyl, 2-piperidinyl, 3-piperidinyl, 4-morpholinyl, 3-morpholinyl, 1,8 diazo-spiro-[4,5]decyl, 1, 7 diazo-spiro-[4,5]decyl, 1,6 diazo-spiro-[4,5]decyl, 2,8 diazo-spiro[4,5]decyl, 2, 7 diazo-spiro[4,5]decyl, 2,6 diazo-spiro[4,5]decyl, 1,8 diazo-spiro-[5,4]decyl, 1,7 diazo-spirotetrahydrofuran-3-yl, tetrahydrothien-2-yl, tetrahydrothien-3-yl, 1-piperazinyl, 2-piperazinyl, and the like.

The term “aryl” preferably refers to an aromatic monocyclic ring containing 6 carbon atoms, an aromatic bicyclic ring system containing 10 carbon atoms or an aromatic tricyclic ring system containing 14 carbon atoms. Examples are phenyl, naphtyl or anthracenyl. The aryl group is optionally substituted.

The term “aralkyl” refers to an alkyl moiety, which is substituted by aryl, wherein alkyl and aryl have the meaning as outlined above. An example is the benzyl radical. Preferably, in this context the alkyl chain comprises from 1 to 8 carbon atoms, i.e. 1, 2, 3, 4, 5, 6, 7, or 8, e.g. methyl, ethyl methyl, ethyl, propyl, iso-propyl, butyl, iso-butyl, sec-butenyl, tert-butyl, pentyl, hexyl, pentyl, octyl. The aralkyl group is optionally substituted at the alkyl and/or aryl part of the group.

The term “heteroaryl” preferably refers to a five or six-membered aromatic monocyclic ring wherein at least one of the carbon atoms are replaced by 1, 2, 3, or 4 (for the five membered ring) or 1, 2, 3, 4, or 5 (for the six membered ring) of the same or different heteroatoms, preferably selected from O, N and S; an aromatic bicyclic ring system wherein 1, 2, 3, 4, 5, or 6 carbon atoms of the 8, 9, 10, 11 or 12 carbon atoms have been replaced with the same or different heteroatoms, preferably selected from O, N and S; or an aromatic tricyclic ring system wherein 1, 2, 3, 4, 5, or 6 carbon atoms of the 13, 14, 15, or 16 carbon atoms have been replaced with the same or different heteroatoms, preferably selected from 0, N and S. Examples are oxazolyl, isoxazolyl, 1,2,5-oxadiazolyl, 1,2,3-oxadiazolyl, pyrrolyl, imidazolyl, pyrazolyl, 1,2,3-triazolyl, thiazolyl, isothiazolyl, 1,2,3,-thiadiazolyl, 1,2,5-thiadiazolyl, pyridinyl, pyrimidinyl, pyrazinyl, 1,2,3-triazinyl, 1,2,4-triazinyl, 1,3,5-triazinyl, 1-benzofuranyl, 2-benzofuranyl, indoyl, isoindoyl, benzothiophenyl, 2-benzothiophenyl, 1H-indazolyl, benzimidazolyl, benzoxazolyl, indoxazinyl, 2, 1-benzosoxazoyl, benzothiazolyl, 1,2-benzisothiazolyl, 2, 1-benzisothiazolyl, benzotriazolyl, quinolinyl, isoquinolinyl, quinoxalinyl, quinazolinyl, quinolinyl, 1,2,3-benzotriazinyl, or 1,2,4-benzotriazinyl.

The term “heteroaralkyl” refers to an alkyl moiety, which is substituted by heteroaryl, wherein alkyl and heteroaryl have the meaning as outlined above. An example is the 2-alklypyridinyl, 3-alkylpyridinyl, or 2-methylpyridinyl. Preferably, in this context the alkyl chain comprises from 1 to 8 carbon atoms, i.e. 1, 2, 3, 4, 5, 6, 7, or 8, e.g. methyl, ethyl methyl, ethyl, propyl, iso-propyl, butyl, iso-butyl, sec-butenyl, tert-butyl, pentyl, hexyl, pentyl, octyl.

The heteroaralkyl group is optionally substituted at the alkyl and/or heteroaryl part of the group.

The terms “alkenyl” and “cycloalkenyl” refer to olefinic unsaturated carbon atoms containing chains or rings with one or more double bonds. Examples are propenyl and cyclohexenyl. Preferably, the alkenyl chain comprises from 2 to 8 carbon atoms, i.e. 2, 3, 4, 5, 6, 7, or 8, e.g. ethenyl, 1-propenyl, 2-propenyl, iso-propenyl, 1-butenyl, 2-butenyl, 3-butenyl, iso-butenyl, sec-butenyl, 1-pentenyl, 2-pentenyl, 3-pentenyl, 4-pentenyl, hexenyl, pentenyl, octenyl. Preferably the cycloalkenyl ring comprises from 3 to 8 carbon atoms, i.e. 3, 4, 5, 6, 7, or 8, e.g. 1-cyclopropenyl, 2-cyclopropenyl, 1-cyclobutenyl, 2-cylcobutenyl, 1-cyclopentenyl, 2-cyclopentenyl, 3-cyclopentenyl, cyclohexenyl, cyclopentenyl, cyclooctenyl.

The term “alkynyl” refers to unsaturated carbon atoms containing chains or rings with one or more triple bonds. An example is the propargyl radical. Preferably, the alkynyl chain comprises from 2 to 8 carbon atoms, i.e. 2, 3, 4, 5, 6, 7, or 8, e.g. ethynyl, 1-propynyl, 2-propynyl, 1-butynyl, 2-butynyl, 3-butynyl, 1-pentynyl, 2-pentynyl, 3-pentynyl, 4-pentynyl, hexynyl, pentynyl, octynyl.

In one embodiment, carbon atoms or hydrogen atoms in alkyl, heteroalkyl, cycloalkyl, aryl, aralkyl, alkenyl, cycloalkenyl, alkynyl radicals may be substituted independently from each other with one or more elements selected from the group consisting of O, S, N or with groups containing one or more elements selected from the group consisting of O, S, N.

Embodiments include alkoxy, cycloalkoxy, arykoxy, aralkoxy, alkenyloxy, cycloalkenyloxy, alkynyloxy, alkylthio, cycloalkylthio, arylthio, aralkylthio, alkenylthio, cycloalkenylthio, alkynylthio, alkylamino, cycloalkylamino, arylamino, aralkylamino, alkenylamino, cycloalkenylamino, alkynylamino radicals.

Other embodiments include hydroxyalkyl, hydroxycycloalkyl, hydroxyaryl, hydroxyaralkyl, hydroxyalkenyl, hydroxycycloalkenyl, hydroxyalinyl, mercaptoalkyl, mercaptocycloalkyk, mercaptoaryl, mercaptoaralkyl, mercaptoalkenyl, mercaptocycloalkenyl, mercaptoalkynyl, aminoalkyl, aminocycloalkyl, aminoaryl, aminoaralkyl, aminoalkenyl, aminocycloalkenyl, aminoalkynyl radicals.

In another embodiment, hydrogen atoms in alkyl, heteroalkyl, cycloalkyl, aryl, aralkyl, alkenyl, cycloalkenyl, alkynyl radicals may be substituted independently from each other with one or more halogen atoms. One radical is the trifluoromethyl radical.

If two or more radicals or two or more residues can be selected independently from each other, then the term “independently” means that the radicals or the residues may be the same or may be different.

As used herein a wording defining the limits of a range of length such as, e. g., “from 1 to 6” means any integer from 1 to 6, i.e. 1, 2, 3, 4, 5 and 6. In other words, any range defined by two integers explicitly mentioned is meant to comprise and disclose any integer defining said limits and any integer comprised in said range.

The term “halo” as used herein refers to a halogen residue selected from the group consisting of F, Br, I, and Cl. Preferably, the halogen is F.

The phrase “protecting group” as used herein, means temporary substituents which protect a potentially reactive functional group from undesired chemical transformations.

The term “amino-protecting group” or “N-terminal protecting group” refers to those groups intended to protect the α-N-terminal of an amino acid or peptide or to otherwise protect the amino group of an amino acid or peptide against undesirable reactions during synthetic procedures. Commonly used N-protecting groups are disclosed in Greene, Protective Groups In Organic Synthesis, (John Wiley & Sons, New York (1981)), which is hereby incorporated by reference. Additionally, protecting groups can be used as pro-drugs which are readily cleaved in vivo, for example, by enzymatic hydrolysis, to release the biologically active parent. α-N-Protecting groups comprise lower alkanoyl groups such as formyl, acetyl (“Ac”), propionyl, pivaloyl, t-butylacetyl and the like; other acyl groups include 2-chloroacetyl, 2-bromoacetyl, trifluoroacetyl, trichloroacetyl, phthalyl, o-nitrophenoxyacetyl, -chlorobutyryl, benzoyl, 4-chlorobenzoyl, 4-bromobenzoyl, 4-nitrobenzoyl and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-ethoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyoxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; arylalkyl groups such as benzyl, triphenylmethyl, benzyloxymethyl, 9-fluorenylmethyloxycarbonyl (Fmoc) and the like and silyl groups such as trimethylsilyl and the like. Still other examples include theyl, succinyl, methoxysuccinyl, subery, adipyl, azelayl, dansyl, benzyloxycarbonyl, methoxyazelaly, methoxyadipyl, methoxysuberyl, and 2,4-dinitrophenyl. The term “linker” as used herein refers to any chemically suitable linker. Preferably, linker are not or only slowly cleaved under physiological conditions. Thus, it is preferred that the linker does not comprise recognition sequences for proteases or recognition structures for other degrading enzymes. Since it is preferred that the compounds of the invention are administered systemically to allow broad access to all compartments of the body and subsequently enrichment of the compounds of the invention wherever in the body the tumor is located, it is preferred that the linker is chosen in such that it is not or only slowly cleaved in blood. The cleavage is considered slowly, if less than 50% of the linkers are cleaved 2 h after administration of the compound to a human patient. Suitable linkers, for example, comprises or consists of optionally substituted alkyl, heteroalkyl, cycloalkyl, cycloheteroalkyl, aryl, heteroaryl, aralkyl, heteroaralyl, alkenyl, heteroalkenyl, cycloalkenyl, cycloheteroalkenyl, alkynyl, sulfonyl, amines, ethers, thioethers phosphines, phosphoramidates, carboxamides, esters, imidoesters, amidines, thioesters, sulfonamides, 3-thiopyrrolidine-2,5-dion, carbamates, ureas, guanidines, thioureas, disulfides, oximes, hydrazines, hydrazides, hydrazones, diaza bonds, triazoles, triazolines, tetrazines, platinum complexes and amino acids, or combinations thereof. Preferably, the linker comprises or consists of 1,4-piperazine, 1,3-propane and a phenolic ether or combinations thereof.

The expression “optionally substituted” refers to a group in which one, two, three or more hydrogen atoms may have been replaced independently of each other by the respective substituents.

As used herein, the term “amino acid” refers to any organic acid containing one or more amino substituents, e.g. α-, β- or γ-amino, derivatives of aliphatic carboxylic acids.

The term “conventional amino acid” refers to the twenty naturally occurring amino acids, and encompasses all stereomeric isoforms, i.e. D, L-, D- and L-amino acids thereof.

The term “N-containing aromatic or non-aromatic mono or bicyclic heterocycle” as used herein refers to a cyclic saturated or unsaturated hydrocarbon compound which contains at least one nitrogen atom as constituent of the cyclic chain.

Illustrative examples of pharmaceutically acceptable salts include but are not limited to: acetate, adipate, alginate, ascorbate, aspartate, benzenesulfonate, benzoate, bicarbonate, bisulfate, bitartrate, borate, bromide, butyrate, calcium edetate, camphorate, camphorsulfonate, camsylate, carbonate, chloride, citrate, clavulanate, cyclopentanepropionate, digluconate, dihydrochloride, dodecylsulfate, edetate, edisylate, estolate, esylate, ethanesulfonate, formate, fumarate, gluceptate, glucoheptonate, gluconate, glutamate, glycerophosphate, glycolylarsanilate, hemisulfate, heptanoate, hexanoate, hexylresorcinate, hydrabamine, hydrobromide, hydrochloride, hydroiodide, 2-hydroxy-ethanesulfonate, hydroxynaphthoate, iodide, isothionate, lactate, lactobionate, laurate, lauryl sulfate, malate, maleate, malonate, mandelate, mesylate, methanesulfonate, methylsulfate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, 3-phenylpropionate, phosphate/diphosphate, picrate, pivalate, polygalacturonate, propionate, salicylate, stearate, sulfate, subacetate, succinate, tannate, tartrate, teoclate, tosylate, triethiodide, undecanoate, valerate, and the like (see, for example, Berge, S. M., et al, “Pharmaceutical Salts”, Journal of Pharmaceutical Science, 1977, 66, 1-19). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide a compound of formula (I). A prodrug is an active or inactive compound that is modified chemically through in vivo physiological action, such as hydrolysis, metabolism and the like, into a compound of this invention following administration of the prodrug to a patient. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme. The suitability and techniques involved in making and using prodrugs are well known by those skilled in the art. For a general discussion of prodrugs involving esters see Svensson and Tunek Drug Metabolism Reviews 16.5 (1988) and Bundgaard Design of Prodrugs, Elsevier (1985).

Examples of a masked carboxylate anion include a variety of esters, such as alkyl (for example, methyl, ethyl), cycloalkyl (for example, cyclohexyl), aralkyl (for example, benzyl, p-methoxybenzyl), and alkylcarbonyloxyalkyl (for example, pivaloyloxymethyl). Amines have been masked as arylcarbonyloxymethyl substituted derivatives which are cleaved by esterases in vivo releasing the free drug and formaldehyde (Bungaard J. Med. Chem. 2503 (1989)). Also, drugs containing an acidic NH group, such as imidazole, imide, indole and the like, have been masked with N-acyloxymethyl groups (Bundgaard Design of Prodrugs, Elsevier (1985)).

Hydroxyl groups have been masked as esters and ethers. EP 0 039 051 (Sloan and Little, Apr. 11, 1981) discloses Mannich base-hydroxamic acid prodrugs, their preparation and use.

Certain compounds of the present invention can exist in unsolvated forms as well as in solvated forms, including hydrated forms. In general, the solvated forms are equivalent to unsolvated forms and are intended to be encompassed within the scope of the present invention.

Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention, while including an unnatural proportions of atomic isotopes at one or more of the atoms that constitute such compounds, still have less than 100% of the molecules including the radioisotopic version of the atom.

The term “pharmaceutical composition” as used in the present application refers to a substance and/or a combination of substances being used for the identification, prevention or treatment of a tissue status or disease. The pharmaceutical composition is formulated to be suitable for administration to a patient in order to prevent and/or treat disease. Further a pharmaceutical composition refers to the combination of an active agent with a carrier, inert or active, making the composition suitable for therapeutic use. Pharmaceutical compositions can be formulated for oral, parenteral, topical, inhalative, rectal, sublingual, transdermal, subcutaneous or vaginal application routes according to their chemical and physical properties. Pharmaceutical compositions comprise solid, semisolid, liquid, transdermal therapeutic systems (TTS). Solid compositions are selected from the group consisting of tablets, coated tablets, powder, granulate, pellets, capsules, effervescent tablets or transdermal therapeutic systems. Also comprised are liquid compositions, selected from the group consisting of solutions, syrups, infusions, extracts, solutions for intravenous application, solutions for infusion or solutions of the carrier systems of the present invention. Semisolid compositions that can be used in the context of the invention comprise emulsion, suspension, creams, lotions, gels, globules, buccal tablets and suppositories.

“Pharmaceutically acceptable” means approved by a regulatory agency of the Federal or a state government or listed in the U.S. Pharmacopeia or other generally recognized pharmacopeia for use in animals, and more particularly in humans.

The term “carrier”, as used herein, refers to a diluent, adjuvant, excipient, or vehicle with which the therapeutic agent is administered. Such pharmaceutical carriers can be sterile liquids, such as saline solutions in water and oils, including those of petroleum, animal, vegetable or synthetic origin, such as peanut oil, soybean oil, mineral oil, sesame oil and the like. A saline solution is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Examples of suitable pharmaceutical carriers are described in “Remington's Pharmaceutical Sciences” by E. W. Martin.

### III. Overview of FAP-Activated Radiopharmaceutical Structures

In some embodiments, the FAP-activated theranostic prodrug is represented by:

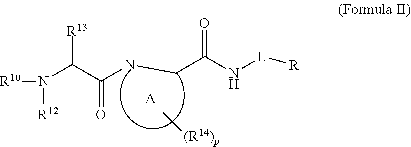

or a pharmaceutically acceptable salt thereof, wherein:
   R represents a ligand-targeted theranostic moiety, including a ligand for binding to a cellular target and one or more of a radioactive moiety and/or a chelating agent for chelating a radioactive moiety;
   A represents a 5 to 8 membered heterocycle ring;
   X is O or S;
   R¹⁰ is an amino terminal blocking group
   R¹² is hydrogen or (C₁-C₆)alkyl;
   R¹³ is hydrogen, a (C₁-C₆)alkyl (which may be straight or branched chain) or a (C1-C6);
   R¹⁴ is, independently for each occurrence, —(C₁-C₆)alkyl, —OH, —NH₂, or halogen;
   p is an integer from 0-6; and
   L is a bond, or after cleavage by FAP to release NH₂-L-R, is a self-eliminating linker;
and wherein
   enzymatic cleavage of the prodrug by fibrolast activation protein (FAP) leads to the release of the ligand-targeted theranostic moiety either as an activated ligand-targeted theranostic moiety (i.e., its pharmacologically active form) or in a form that is readily metabolized to its active form; and
   when released from the prodrug by FAP cleavage, the activated ligand-targeted theranostic moiety binds to the cellular target with a Kd for binding to the cellular target that is less (i.e., has a higher affinity for the cellular target) than the Kd for the prodrug binding to the cellular target.

In certain preferred embodiments of Formula II, R¹² is H.

In certain embodiments of the structure of Formula II, the R¹⁰ forms an amide OR thioamide with the nitrogen to which it is attached, and the prodrug is represented in the formula

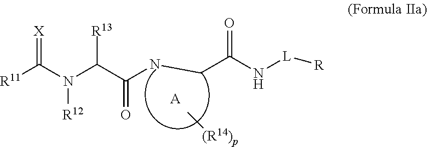

or a pharmaceutically acceptable salt thereof, wherein:
   X is O or S;
   R¹¹—(C═X) taken together represents an acyl N-terminal blocking group; or
   R¹¹ is —(C₁-C₁₀)alkyl, —(C₁-C₁₀)alkoxy, —(C₁-C₁₀)alkyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, -aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio; or
   R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁-C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
   AA is, independently for each occurrence, an amino acid residue;
   n is integer from 1 to 5, and
   R, A, R¹² R¹³ R¹⁴ p and L are as set forth for Formula II.

In certain preferred embodiments of Formula IIa: X is O; and/or R¹² is H. In certain preferred embodiments X is O and R¹² is H.

In certain preferred embodiments, X is O, and R¹¹ is —(C₁-C₁₀)alkyl-CO₂H, —(C₁-C₁₀)alkenyl-CO₂H or —(C₁-C₁₀)aryl-CO₂H.

In certain preferred embodiments, X is O, and —C(═O)—R¹¹ forms an acyl of a carboxylic acid, such as, to illustrate, a formyl, acetyl, propionyl, butryl, oxalyl, malonyl, succinyl, glutaryl, adipoyl, acryloyl, maleoyl, fumaroyl, glycoloyl, lactoyl, pyruvoyl, glyceroyl, maloyl, oxaloacetyl, benzoyl, trifluoroacetyl or methoxysuccinyl group.

In certain preferred embodiments, X is O, and R¹¹ is —(CH₂)ₘ—R^{11a}, where R^{11a} is a 5-10-membered aryl or heteroaryl group, and m is an integer from 1 to 6. In certain embodiments, m is 1 or 2. In certain embodiments, R^{11a} is a 6-membered aryl or heteroaryl group.

In certain embodiments, the FAP-activated theranostic prodrug is represented:

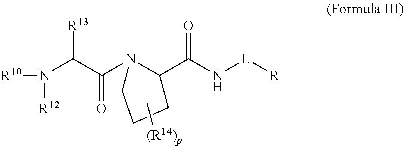

or a pharmaceutically acceptable salt thereof, wherein, R, R¹⁰, R¹², R¹³, R¹⁴, L, X and p are as defined for Formula II above.

In certain preferred embodiments of Formula III, R¹² is H.

In certain embodiments of the structure of Formula III, R¹⁰ forms an amide OR thioamide with the nitrogen to which it is attached, and the prodrug is represented by formula IIIa

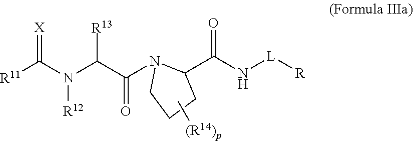

or a pharmaceutically acceptable salt thereof, wherein:
   X is O or S;
   R¹¹—(C═X) taken together represents an acyl N-terminal blocking group; or
   R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, -aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl,
   wherein R¹¹ is optionally substituted with one or more substituents independently selected from the group consisting of halo, hydroxy, alkoxy, carboxy, cyano, amino, nitro, and thio, or
   R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁-C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
   AA is, independently for each occurrence, an amino acid residue;
   n is integer from 1 to 5, and
   R, R¹² R¹³ R¹⁴ p and L are as set forth for Formula II.

In certain preferred embodiments of Formula IIIa: X is O; and/or R¹² is H. In certain preferred embodiments, X is O and R¹² is H.

In certain preferred embodiments, X is O, and R¹¹ is —(C₁-C₁₀)alkyl-CO₂H, —(C₁-C₁₀)alkenyl-CO₂H or —(C₁-C₁₀)aryl-CO₂H.

In certain preferred embodiments, X is O, and —C(═O)—R¹¹ forms an acyl of a carboxylic acid, such as, to illustrate, a formyl, acetyl, propionyl, butryl, oxalyl, malonyl, succinyl, glutaryl, adipoyl, acryloyl, maleoyl, fumaroyl, glycoloyl, lactoyl, pyruvoyl, glyceroyl, maloyl, oxaloacetyl, benzoyl, trifluoroacetyl or methoxysuccinyl group.

In certain preferred embodiments, X is O, and R¹¹ is —(CH₂)ₘR^{11a}, where R^{11a} is a 5-10-membered aryl or heteroaryl group, and m is an integer from 1 to 6. In certain embodiments, m is 1 or 2. In certain embodiments, R^{11a} is a 6-membered aryl or heteroaryl group.

In certain embodiments, the FAP-activated theranostic prodrug is represented by Formula IV:

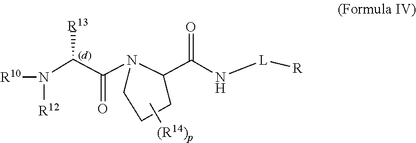

or a pharmaceutically acceptable salt thereof, wherein, R¹³ is a (C₁-C₆)alkyl (which may be straight or branched chain) or a (C₁-C₆), and R, R¹⁰, R¹², R¹³, R¹⁴, L, X and p are as defined for Formula II above.

In certain preferred embodiments of Formula IV: R¹³ is methyl; p is zero; and/or R¹² is H. In certain preferred embodiments R¹³ is methyl, p is zero (i.e., R¹⁴ is absent) and R¹² is H.

In certain embodiments of the structure of Formula IV, R¹⁰ forms an amide OR thioamide with the nitrogen to which it is attached, and the prodrug is represented in the formula IVa

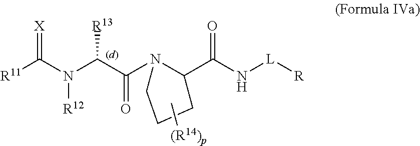

or a pharmaceutically acceptable salt thereof, wherein:
   X is O or S;
   R¹¹—(C═X) taken together represents an acyl N-terminal blocking group; or
   R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, -aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl,
   wherein R¹¹ is optionally substituted with one or more substituents independently selected from the group consisting of halo, hydroxy, alkoxy, carboxy, cyano, amino, nitro, and thio, or
   R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁—C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
   AA is, independently for each occurrence, an amino acid residue;
   n is integer from 1 to 5, and
   R, R¹² R¹³ R¹⁴ p and L are as set forth for Formula II.

In certain preferred embodiments of Formula IVa: R¹³ is methyl; X is O; p is zero; and/or R¹² is H. In certain preferred embodiments R¹³ is methyl, X is O, p is zero and R¹² is H.

In certain preferred embodiments, X is O, and R¹¹ is —(C₁-C₁₀)alkyl-CO₂H, —(C₁-C₁₀)alkenyl-CO₂H or —(C₁-C₁₀)aryl-CO₂H.

In certain preferred embodiments, X is O, and —C(═O)—R¹¹ forms an acyl of a carboxylic acid, such as, to illustrate, a formyl, acetyl, propionyl, butryl, oxalyl, malonyl, succinyl, glutaryl, adipoyl, acryloyl, maleoyl, fumaroyl, glycoloyl, lactoyl, pyruvoyl, glyceroyl, maloyl, oxaloacetyl, benzoyl, trifluoroacetyl or methoxysuccinyl group.

In certain preferred embodiments, X is O, and R¹¹ is —(CH₂)ₘR^{11a}, where R^{11a} is a 5-10-membered aryl or heteroaryl group, and m is an integer from 1 to 6. In certain embodiments, m is 1 or 2. In certain embodiments, R^{11a} is a 6-membered aryl or heteroaryl group.

In certain embodiments, the FAP-activated radiopharmaceutical is represented:

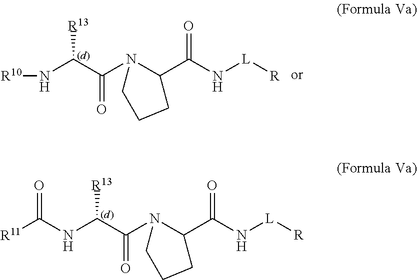

wherein,
   R, R¹⁰, R¹³ and L are as set forth for Formula II, and
   R¹¹—(C═O) taken together represents an acyl N-terminal blocking group; or
   R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, -aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl,
   wherein R¹¹ is optionally substituted with one or more substituents independently selected from the group consisting of halo, hydroxy, alkoxy, carboxy, cyano, amino, nitro, and thio, or
   R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁-C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
   AA is, independently for each occurrence, an amino acid residue;
   n is integer from 1 to 5, and
   R¹³ is a (C₁-C₆)alkyl (which may be straight or branched chain) or a (C₁-C₆).

In certain preferred embodiments of Formulas V and Va, R¹³ is methyl.

In certain preferred embodiments, X is O, and R¹¹ is —(C₁-C₁₀)alkyl-CO₂H, —(C₁-C₁₀)alkenyl-CO₂H or —(C₁-C₁₀)aryl-CO₂H.

In certain preferred embodiments, X is O, and —C(═O)—R¹¹ forms an acyl of a carboxylic acid, such as, to illustrate, a formyl, acetyl, propionyl, butryl, oxalyl, malonyl, succinyl, glutaryl, adipoyl, acryloyl, maleoyl, fumaroyl, glycoloyl, lactoyl, pyruvoyl, glyceroyl, maloyl, oxaloacetyl, benzoyl, trifluoroacetyl or methoxysuccinyl group.

In certain preferred embodiments, X is O, and R¹¹ is —(CH₂)ₘR^{11a}, where R^{11a} is a 5-10-membered aryl or heteroaryl group, and m is an integer from 1 to 6. In certain embodiments, m is 1 or 2. In certain embodiments, R^{11a} is a 6-membered aryl or heteroaryl group.

In certain embodiments, the FAP-activated theranostic prodrug is represented by formula VI:

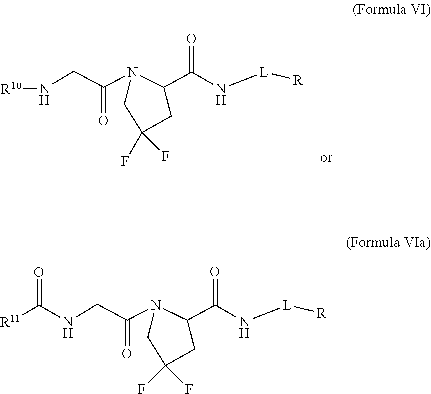

wherein,
   R, R¹⁰ and L are as set forth for Formula II, and
   R¹¹—(C═O) taken together represents an acyl N-terminal blocking group; or
   R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, -aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl,
   wherein R¹¹ is optionally substituted with one or more substituents independently selected from the group consisting of halo, hydroxy, alkoxy, carboxy, cyano, amino, nitro, and thio, or
   R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁-C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
   AA is, independently for each occurrence, an amino acid residue; and
   n is integer from 1 to 5.

In certain preferred embodiments, X is O, and R¹¹ is —(C₁-C₁₀)alkyl-CO₂H, —(C₁-C₁₀)alkenyl-CO₂H or —(C₁-C₁₀)aryl-CO₂H.

In certain preferred embodiments, X is O, and —C(═O)—R¹¹ forms an acyl of a carboxylic acid, such as, to illustrate, a formyl, acetyl, propionyl, butryl, oxalyl, malonyl, succinyl, glutaryl, adipoyl, acryloyl, maleoyl, fumaroyl, glycoloyl, lactoyl, pyruvoyl, glyceroyl, maloyl, oxaloacetyl, benzoyl, trifluoroacetyl or methoxysuccinyl group.

In certain preferred embodiments, X is O, and R¹¹ is —(CH₂)ₘR^{11a}, where R^{11a} is a 5-10-membered aryl or heteroaryl group, and m is an integer from 1 to 6. In certain embodiments, m is 1 or 2. In certain embodiments, R^{11a} is a 6-membered aryl or heteroaryl group.

In still other embodiments, the FAP-activated theranostic prodrug is represented by formula VII:

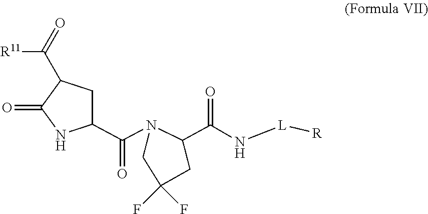

wherein, R, and L are as defined for Formula II above, and —C(═O)R¹¹ forms an acyl group.

In certain embodiments of the above structures II through VII, —C(═X)R¹¹ or —C(═O)R¹¹ form an acyl group.

In certain embodiments, the acyl group is selected from the group consisting of aryl(C₁-C₆)acyl and heteroaryl(C₁-C₆)acyl.

In certain embodiments, the aryl(C₁-C₆)acyl is a (C₁-C₆)acyl substituted with an aryl selected from the group consisting of benzyl, naphthalenyl, phenanthrenyl, phenolyl, and anilinyl.

In certain embodiments, the aryl(C₁-C₆)acyl is a (C₁)acyl substituted with an aryl selected from the group consisting of benzyl, naphthalenyl, phenanthrenyl, phenolyl, and anilinyl.

In certain embodiments, the acyl group is a heteroaryl(C₁-C₆)acyl.

In certain embodiments, the heteroaryl(C₁-C₆)acyl is a (C₁-C₆)acyl substituted with a heteroaryl selected from the group consisting of pyrryl, furyl, thiophenyl (a/k/a thienyl), imidazolyl, oxazolyl, thiazolyl, triazolyl, pyrazolyl, pyridinyl, pyrazinyl, pyridazinyl, and pyrimidinyl.

In certain embodiments, the heteroaryl(C₁-C₆)acyl is a (C₁)acyl substituted with a heteroaryl selected from the group consisting of pyrryl, furyl, thiophenyl (a/k/a thienyl), imidazolyl, oxazolyl, thiazolyl, triazolyl, pyrazolyl, pyridinyl, pyrazinyl, pyridazinyl, and pyrimidinyl.

In certain embodiments, the FAP substrate moeity comprises a third amino position, optionally N-terminal to (d)-Ala (or other (d)-amino acid in that position and formed by R³), and optionally wherein the amino acid at the third amino acid position is serine or threonine.

a. Self-Eliminating Linkers

In certain embodiments, the FAP substrate moiety is linked to the ligand-targeted theranostic moiety via a self-eliminating linker (L in the above formula). Upon cleavage of the FAP substrate moiety by FAPα, the activated ligand-targeted theranostic moiety is then released upon elimination of the self-eliminating linker.

A self-eliminating moiety may be defined as a bifunctional chemical group that is capable of covalently linking together two spaced chemical moieties into a normally stable molecule, releasing one of the spaced chemical moieties from the molecule by means of enzymatic cleavage; and following enzymatic cleavage, spontaneously cleaving from the remainder of the bifunctional chemical group to release the other of said spaced chemical moieties. Therefore, in some embodiments, the self-eliminating moiety is covalently linked at one of its ends, directly or indirectly through a spacer unit, to the ligand by an amide bond and covalently linked at its other end to a chemical reactive site (functional group) pending from the therapeutic moiety.

A therapeutic conjugate is generally stable in circulation, or at least that should be the case in the absence of an enzyme capable of cleaving the amide bond between the substrate recognition sequence (FAPα-cleavable linker) and the self-eliminating moiety. Upon exposure of a therapeutic conjugate to a suitable enzyme (FAPα), the amide bond is cleaved initiating a spontaneous self-eliminating reaction resulting in the cleavage of the bond covalently linking the self-eliminating moiety to the therapeutic moiety, to thereby effect release of the free therapeutic moiety in its underivatized or pharmacologically active form. The self-eliminating moiety in conjugates either incorporate one or more heteroatoms and thereby provides improved solubility, improves the rate of cleavage and decreases propensity for aggregation of the conjugate.

In some embodiments, L is a benzyl oxy carbonyl group. In other embodiments, the self-eliminating linker L is —NH—(CH₂)₄—C(═O)— or —NH—(CH₂)₃—C(═O)—. In yet other embodiments, the self-eliminating linker L is p-aminobenzyloxycarbonyl (PABC). In still other embodiments, the self-eliminating linker L is 2,4-bis(hydroxymethyl)aniline.

The therapeutic conjugates of the present disclosure can employ a heterocyclic self-eliminating moiety covalently linked to the therapeutic moiety and the cleavable substrate recognition sequence. A self-eliminating moiety may be defined as a bifunctional chemical group which is capable of covalently linking together two spaced chemical moieties into a normally stable molecule, releasing one of said spaced chemical moieties from the molecule by means of enzymatic cleavage; and following said enzymatic cleavage, spontaneously cleaving from the remainder of the bifunctional chemical group to release the other of said spaced chemical moieties. In accordance with the present disclosure, the self-eliminating moiety may be covalently linked at one of its ends, directly or indirectly through a spacer unit, to the ligand by an amide bond and covalently linked at its other end to a chemical reactive site (functional group) pending from the drug. The derivatization of the therapeutic moiety with the self-eliminating moiety may render the drug less pharmacologically active (e.g. less toxic) or not active at all until the drug is cleaved.

The therapeutic conjugate is generally stable in circulation, or at least that should be the case in the absence of an enzyme capable of cleaving the amide bond between the substrate recognition sequence and the self-eliminating moiety. However, upon exposure of the therapeutic conjugate to a suitable enzyme, the amide bond is cleaved initiating a spontaneous self-eliminating reaction resulting in the cleavage of the bond covalently linking the self-eliminating moiety to the drug, to thereby effect release of the free therapeutic moiety in its underivatized or pharmacologically active form.

The self-eliminating moiety in conjugates of the present disclosure, in some embodiments, either incorporate one or more heteroatoms and thereby provides improved solubility, improves the rate of cleavage and decreases propensity for aggregation of the conjugate. These improvements of the heterocyclic self-eliminating linker constructs of the present disclosure over non-heterocyclic, PAB-type linkers may result in surprising and unexpected biological properties such as increased efficacy, decreased toxicity, and more desirable pharmacokinetics.

In some embodiments, L is a benzyloxycarbonyl group.

In some embodiments, L is

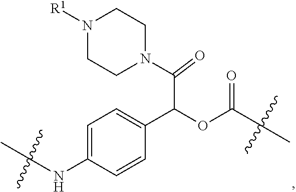

wherein R¹ is hydrogen, unsubstituted or substituted C₁₋₃ alkyl, or unsubstituted or substituted heterocyclyl. In some embodiments, R¹ is hydrogen. In some instances, R¹ is methyl.

In some embodiments, L is selected from

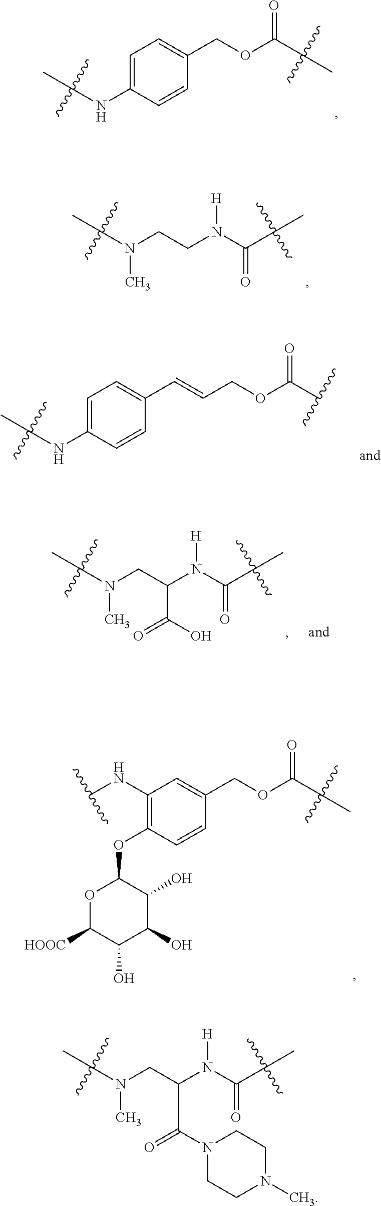

In some embodiments, the self-eliminating moiety L is selected from

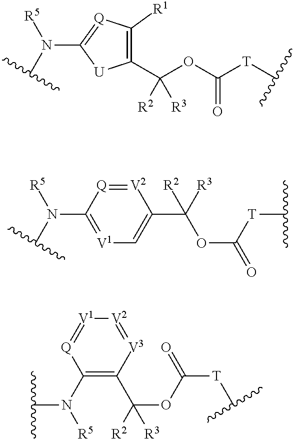

wherein
U is O, S or NR⁶;
Q is CR⁴ or N;
V¹, V² and V³ are independently CR⁴ or N provided that for formula II and III at least one of Q, V¹ and V² is N;
T is NH, NR⁶, O or S pending from said therapeutic moiety;
R¹, R², R³ and R⁴ are independently selected from H, F, Cl, Br, I, OH, —N(R⁵)₂, —N(R⁵)₃⁺, C₁-C₈ alkylhalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ halosubstituted alkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀aryl, C₆-C₂₀ substituted aryl, C₁-C₂₀ heterocycle, and C₁-C₂₀ substituted heterocycle; or when taken together, R² and R³ form a carbonyl (═O), or spiro carbocyclic ring of 3 to 7 carbon atoms; and
R⁵ and R⁶ are independently selected from H, C₁-C₈ alkyl, C₁-C₈substituted alkyl, C₂-C₈ alkenyl, C₂-C₈ substituted alkenyl, C₂-C₈alkynyl, C₂-C₈ substituted alkynyl, C₆-C₂₀aryl, C₆-C₂₀ substituted aryl, C₁-C₂₀ heterocycle, and C₁-C₂₀ substituted heterocycle;
where C₁-C₈ substituted alkyl, C₂-C₈ substituted alkenyl, C₂-C₈substituted alkynyl, C₆-C₂₀ substituted aryl, and C₂-C₂₀ substituted heterocycle are independently substituted with one or more substituents selected from F, Cl, Br, I, OH, —N(R⁵)₂, —N(R⁵)₃⁺, C₁-C₈ alkylhalide, carboxylate, sulfate, sulfamate, sulfonate C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate.

It will be understood that when T is NH, it is derived from a primary amine (—NH2) pending from the therapeutic moiety (prior to coupling to the self-eliminating moiety) and when T is N, it is derived from a secondary amine (—NH—) from the therapeutic moiety (prior to coupling to the self-eliminating moiety). Similarly, when T is O or S, it is derived from a hydroxyl (—OH) or sulfhydryl (—SH) group respectively pending from the therapeutic moiety prior to coupling to the self-eliminating moiety.

In some embodiments, the self-eliminating linker L is —NH—(CH₂)₄—C(═O)— or —NH—(CH₂)₃—C(═O)—.

In some embodiments, the self-eliminating linker L is p-aminobenzyloxycarbonyl (PABC).

In some embodiments, the self-eliminating linker L is 2,4-bis(hydroxymethyl)aniline.

Other examples of self-eliminating linkers that are readily adapted for use in therapeutic conjugates described herein are taught in, for example, U.S. Pat. No. 7,754,681; WO 2012/074693A1; U.S. Pat. No. 9,089,614; EP 1,732,607; WO 2015/038426A1 (all of which are incorporated by reference); Walther et al. “Prodrugs in medicinal chemistry and enzyme prodrug therapies” Adv Drug Deliv Rev. 2017 Sep. 1; 118:65-77; and Tranoy-Opalinski et al. “Design of self-eliminating linkers for tumour-activated prodrug therapy”, Anticancer Agents Med Chem. 2008 August; 8(6):618-37; the teachings of each of which are incorporated by reference herein.

Yet other non-limiting examples of self-eliminating linkers for use in accordance with the present disclosure are described in International Publication No. WO 2019/236567, published Dec. 12, 2019, incorporated by reference herein.

b. Targeting Moiety

In certain embodiments, the ligand-targeted theranostic moiety (R) is represented by

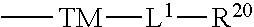

wherein:
TM represents a targeting ligand moiety that selectively binds to a cell surface feature on a target cell;
L¹ represents a bond or a linker; and
R²⁰ represents a radioactive moiety, a chelating agent, a fluorescent moeity, a photoacoustic reporting molecule, a Raman-active reporting molecule, a contrast agent, or a detectable nanoparticle.

For instance, the ligand targeting moiety TM can be a moiety that selectively binds to a cell surface feature on a tumor cell, or a tumor stromal cell.

For instance, the ligand targeting moiety TM can be a moiety that selectively binds to a protein, a carbohydrate or a lipid (such as a glycolipid) on a target cell.

In certain embodiments, the cell surface feature internalizes the ligand-targeted theranostic moiety when it is bound to the cell surface feature.

In certain embodiments, the ligand targeting moiety selectively binds to a protein on the target cell. In certain embodiments, the protein on the target cell is a receptor.

In certain embodiments, the receptor is a G protein coupled receptor (GPCR), such as a gastrin-releasing peptide receptor (such as a bombesin receptor like BB1, BB2 or BB3), calcitonin receptor, oxytocin receptor, a somatostatin receptor (such as somatostatin receptor subtype 2), a melanocortin receptor (e.g., MC1R), a cholecystokinin receptor (such as a cholecystokinin B receptor), a neurotensin receptor or a Neuropeptide Y receptor.

In other embodiments, the receptor is a growth factor receptor, such as an epidermal growth factor receptor (e.g., ErbB1, ErbB2, ErbB3 or ErbB4), an insulin growth factor receptor (e.g., IGFR1 or IGFR2), a TGFβ receptor (e.g., TGFβR1 or TGFβR2), a VEGF receptor (e.g., VEGFR1, VEGFR2, VEGFR3 or VEGFR4), a PDGF receptor (e.g., PDGFRα or PDGFRβ), or and FGF receptor (e.g., FGFR1, FGFR2, FGFR3 or FGFR4).

In certain embodiments, the receptor binding moiety binds to folate receptor a, and can be a folate receptor ligand, such as folic acid or folic acid analogs (such as etarfolatide, vintafolide, leucovorin and methotrexate).

In certain instances, ligand targeting moiety can be selected to bind to an integrin. In certain embodiments, the ligand targeting moiety binds to integrin αᵥβ₃.

In certain embodiments, the ligand targeting moiety can be selected to bind to an N-acetyl-L-aspartyl-L-glutamate peptidase, such as prostate-specific membrane antigen (PSMA).

The ligand targeting moiety can itself have pharmacological activity in and of itself, or can be inert and simply serve the purpose of delivering the ligand-targeted theranostic moiety to (and preferably into) the cell expressing the receptor.

In certain embodiments, the ligand targeting moiety is a somatostatin or a somatostatin analogs, such as octreotate, octreotide or pentetreotide.

In certain embodiments, the ligand targeting moiety binds to αIIbβ3, and can be an αIIbβ3-targeted ligand, such as RGD or an RGD analog (i.e., dimer or multimeric analog), including illustrative cyclic RGD peptides like cyclo(-Arg-Gly-Asp-D-Phe Val-) [“c(RGDfV)” ], c(RGDfK), c(RGDfC), c(RADfC), c(RADfK), c(RGDfE), c(RADfE), RGDSK, RADSK, RGDS, c(RGDyC), c(RADyC), c(RGDyE), c(RGDyK), c(RADyK) and H-E[c(RGDyK)]2, EMD 12194, DMP728, DMP757 and SK&F107260.

To further illustrate, in certain embodiments the ligand targeting moiety binds to prostate-specific membrane antigen (PSMA). For example, the ligand-targeted theranostic moiety (R) in the above structures can be represented as

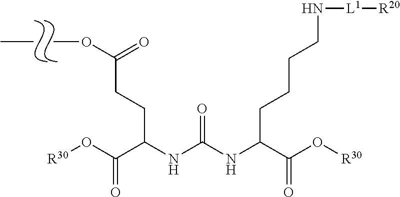

wherein
L¹ represents a bond or a linker;
R²⁰ represents a radioactive moiety, a chelating agent, a fluorescent moeity, a photoacoustic reporting molecule, a Raman-active reporting molecule, a contrast agent, or a detectable nanoparticle; and
R³⁰ represents, independently for each occurrence, a hydrogen or a lower alkyl.

In certain embodiments, L¹ represents a linker. In certain embodiments, the linker L¹ is selected to provide for some hydrophobic contacts with PSMA. In certain embodiments, the linker L¹ is selected to provide for some hydrophobic contacts with PSMA.

In certain embodiments, -L¹-R²⁰ is represented by

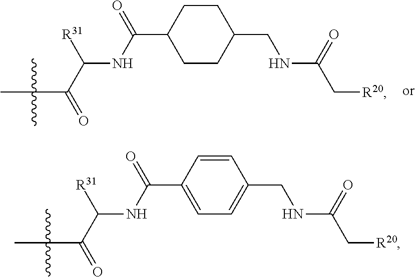

where R²⁰ is as defined above, and R³¹ is —(CH₂)ₚ-aryl or is —(CH₂)ₚ-heteroaryl, and p is 0, 1, 2, 3 or 4. In certain embodiments, p is 1 or 2, and preferably p is 1. In certain embodiments, R³¹ is —CH₂-aryl where the aryl group is a C₆ to C₁₂ aryl, and is a monocyclic or bicyclic fused ring. In certain preferred embodiments, R³¹ is —CH₂-napthalene.

In certain embodiments, R²⁰ is a chelator, which may include a chelated radioisotope.

In certain embodiments, -L¹-R²⁰ is represented by

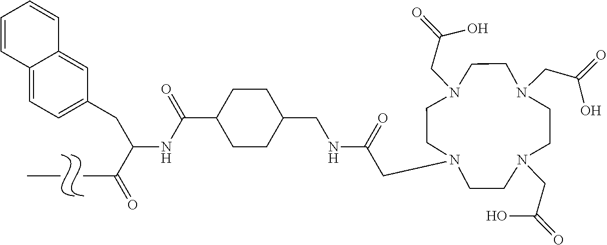

or a radioisotope chelated formulation thereof.

In certain embodiments, R²⁰ is a F¹⁸ containing moiety. To illustrate, -L¹-R²⁰ can be selected from

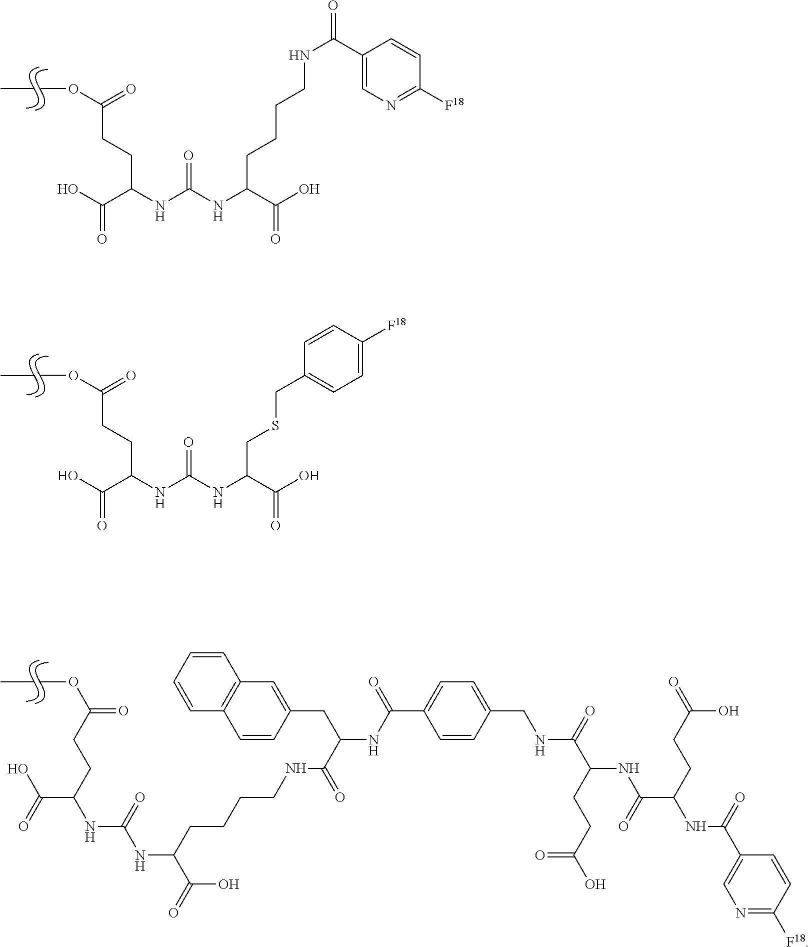

As an additional illustrate, in certain embodiments the ligand targeting moiety binds to folate receptor. For example, the ligand-targeted theranostic moiety (R) includes folic acid or a folic acid analog, such as can be represented as one of

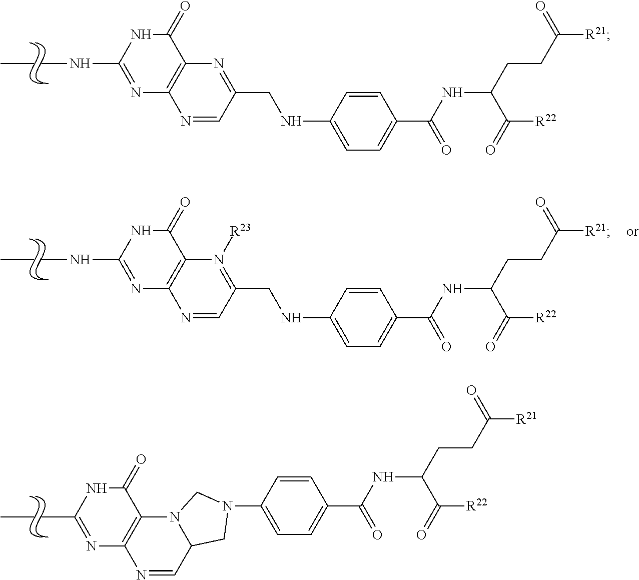

wherein
R²¹ represents H, and R²² represents —NH—(CH₂)_{q}—R²⁰, —NH—(CH₂)_{q}—NH—C(O)—(CH₂)_{q}—R²⁰ or —NH—(CH₂)_{q}—C(O)—(CH₂)_{q}—R²⁰; or
R²² represents H, and R²¹ represents —NH—(CH₂)_{q}—R²⁰ or —NH—(CH₂)_{q}—C(O)—(CH₂)_{q}—R²⁰; or
one of R²¹ or R²² represents H, and the other is selected from the group

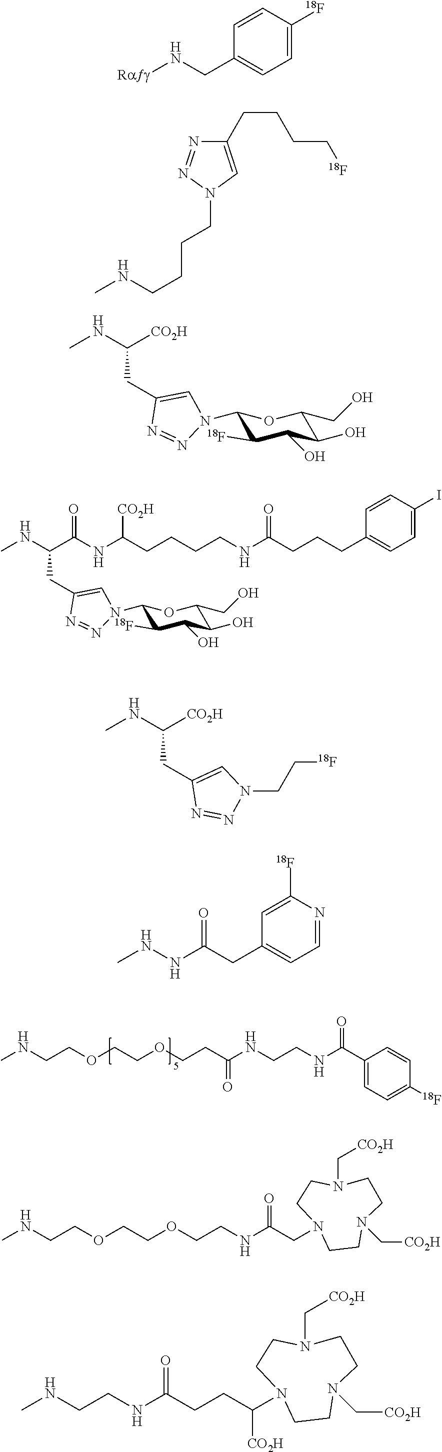

R²³ represents H, —CH₃, —CH₂CH₃, or —CO₂H;
R²⁰ represents a radioactive moiety, a chelating agent, a fluorescent moeity, a photoacoustic reporting molecule, a Raman-active reporting molecule, a contrast agent, or a detectable nanoparticle; and
q, independently for each occurrence, is 0, 1, 2, 3 or 4.

In certain embodiments, R²⁰ represents a chelating moiety, R²¹ represents —NH—CH₂—R²⁰, —NH—CH₂—C(O)—R²⁰, —NH—C(O)—CH₂—R²⁰, —NH—CH₂—C(O)—CH₂—R²⁰ or —NH—(CH₂)₂—NH—C(O)—CH₂—R²⁰ and R²² represents H.

In certain embodiments, R²⁰ represents a chelating moiety, R²¹ represents H, and R²² represents —NH—CH₂—R²⁰, —NH—CH₂—C(O)—R²⁰, —NH—C(O)—CH₂—R²⁰, —NH—CH₂—C(O)—CH₂—R²⁰, or —NH—(CH₂)₂—NH—C(O)—CH₂—R²⁰.

For example, the ligand-target theranostic moiety (R) can be

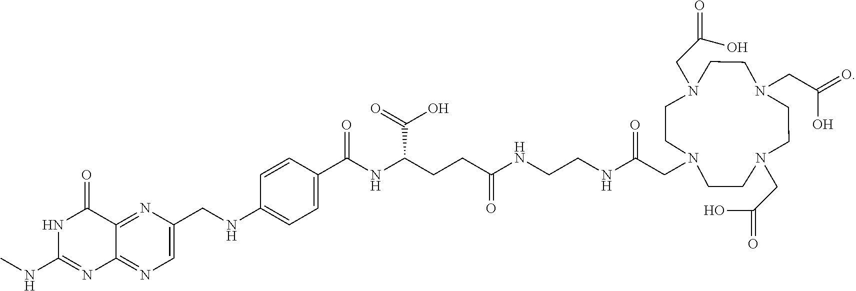 [SMILES: CNc1nc2ncc(CNc3ccc(C(=O)N[C@@H](CCC(=O)NCCNC(=O)CN4CCN(CC(=O)O)CCN(CC(=O)O)CCN(CC(=O)O)CC4)C(=O)O)cc3)nc2c(=O)[nH]1]

In still other embodiments, the ligand-targeted theranostic moiety (R) includes folic acid or a folic acid analog which is directly labeled with a radioistope, such as

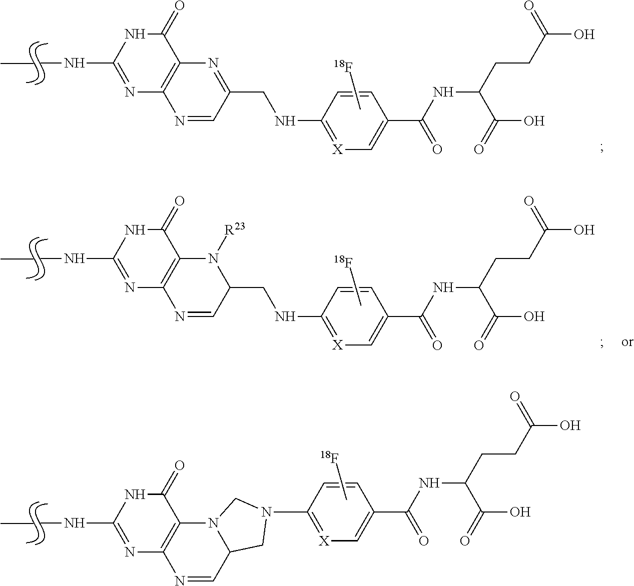

wherein R²³ represents H, —CH₃, —CH₂CH₃, or —CO₂H and X represents CR⁴⁰ or N, wherein R⁴⁰ is H or lower alkyl.

In certain embodiments the ligand targeting moiety binds to a somatostatin receptor (such as somatostatin receptor subtype 2). For example, the ligand-targeted theranostic moiety (R) can include somatostatin or a somatostatin analog. Examples of somatostatin folic acid or a folic acid analog. Examples of somatostatin analogs include octreotide, octreotate, lanreotide, vapreotide, pasireotide, seglitide, benereotide, KE-108, SDZ-222-100, Sst3-ODN-8, CYN-154806, JR11, J2156, SRA-880, ACQ090, P829, SSTp-58, SSTp-86, BASS and somatoprim.

In cerain embodiments, the somatostatin anlog is a somatostatin receptor agonist.

For example, the ligand-targeted theranostic moiety (R) includes can be represented as one of

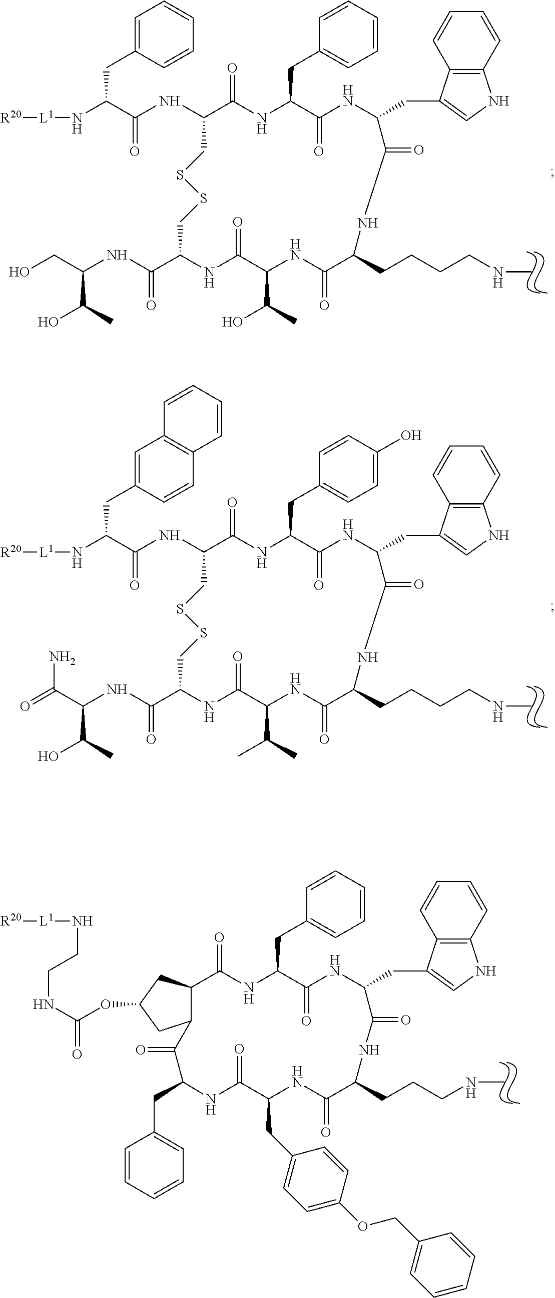

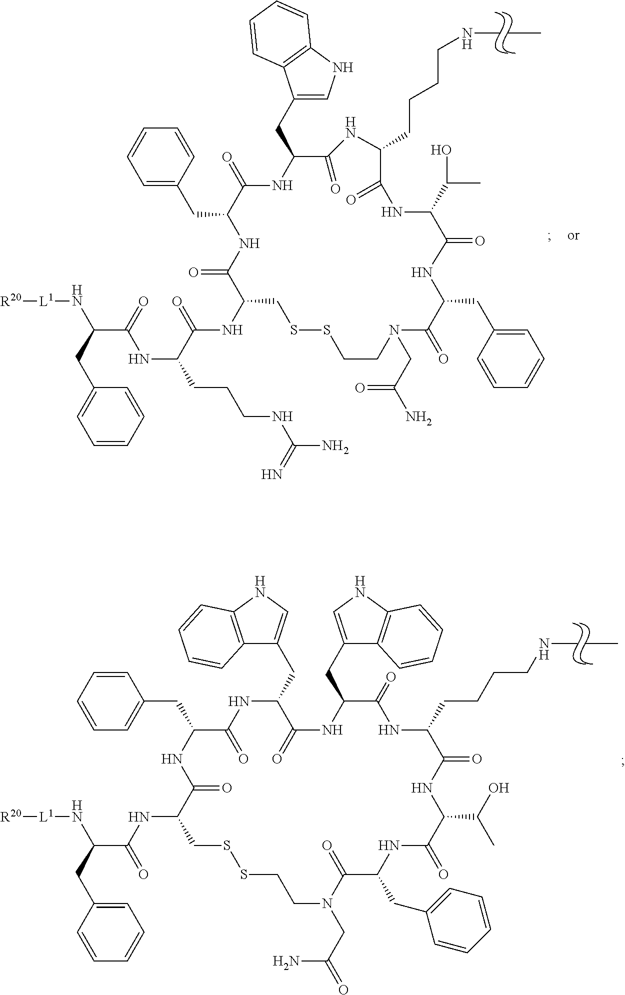

wherein
   L¹ represents a bond or a linker;
   R²⁰ represents a radioactive moiety, a chelating agent, a fluorescent moeity, a photoacoustic reporting molecule, a Raman-active reporting molecule, a contrast agent, or a detectable nanoparticle;

In certain embodiments, R²⁰ is a chelating moeity. To illustrate, R can be a DOTA-octreotate, such as

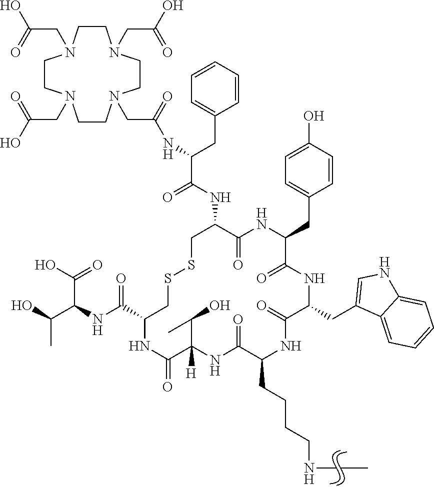

R can also be a (DOTA⁰-Phe¹-Tyr³) octreotide, such as

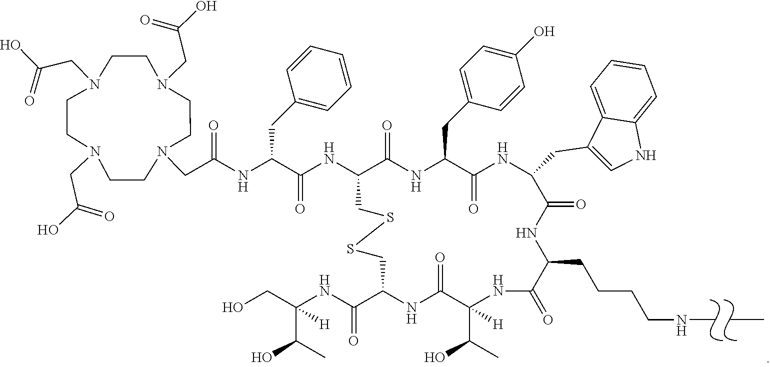

In certain embodiments, R²⁰ is moeity including an ¹⁸F group.

R can be a NOTA-octreotide ([¹⁸F]AlF-NOTA-octreotide shown below), such as

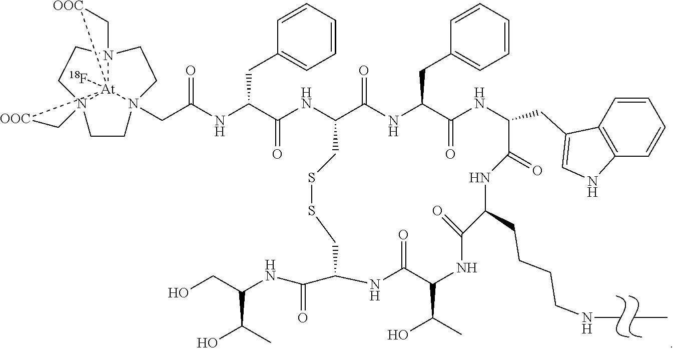

Alternatively, the ¹⁸F can be a substituent directly on the somatostatin or somatostatin analog, or part of a non-chelating tracer moiety, such as when R²⁰-L¹- is

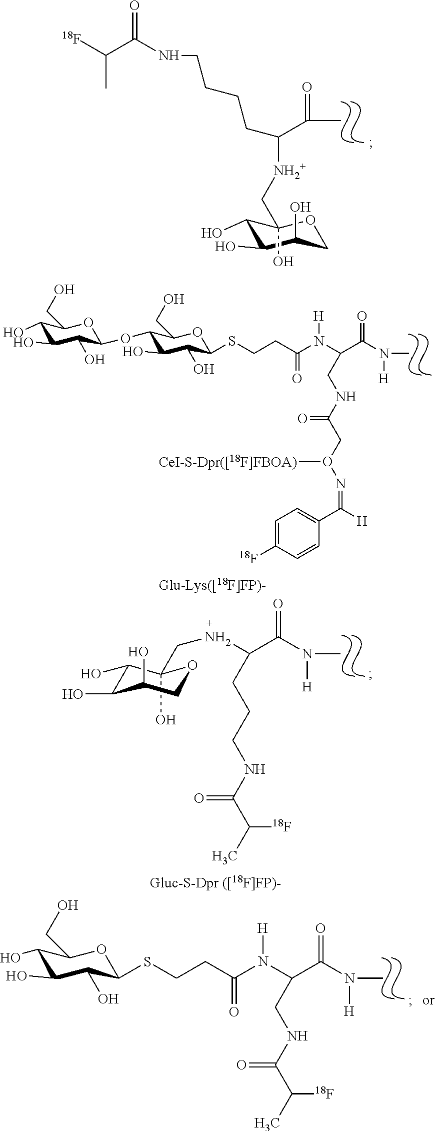

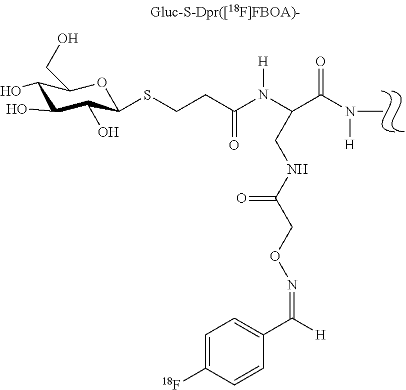

In still other embodiments, the ligand targeting moiety can be selected from bombesin analogs, calcitonin analogs, oxytocin analogs, EGF analogs, α-melanocyte-stimulating hormone analogs, minigastrin analogs, neurotesin analogs, and neuropeptide Y (NPY) analogs.

c. Radioisotopes, Chelators and Other Theranostic Labels

In certain embodiments, the ligand (R in Formulas I, II and III) includes a radioactive moiety, wherein the radioactive moiety includes a fluorescent isotope, a radioisotope, a radioactive drug or combinations thereof. Preferably, the radioactive moiety includes a radioisotope selected from the group consisting of alpha radiation emitting isotopes, beta radiation emitting isotopes, gamma radiation emitting isotopes, Auger electron emitting isotopes, X-ray emitting isotopes, fluorescence emitting isotopes.

The radioactive isotope can be selected to enable imaging and/or radiotherapy.

The radioactive isotopes may include radioactive metal or semi-metal isotopes. Preferably, the radioactive isotopes are water soluble metal cations.

Exemplary radioactive isotopes include ¹⁸F, ⁴³K, ⁴⁷Sc, ⁵¹Cr, ⁵⁷Co, ⁵⁸Co, ⁵⁹Fe, ⁶⁴Cu, ⁶⁷Cu, ⁶⁷Ga, ⁶⁸Ga, ⁷¹Ge, ⁷²As, ⁷²Se, ⁷⁵Br, ⁷⁶Br, ⁷⁷As, ⁷⁷Br, ⁸¹Rb, ⁸⁸Y, ⁹⁰Y, ⁹⁷Ru, ^{99m}Tc, ¹⁰⁰Pd, ^{101m}Rh, ¹⁰³Pb, ¹⁰⁵Rh, ¹⁰⁹Pd, ¹¹¹Ag, ¹¹¹In, ¹¹³In, ¹¹⁹Sb ¹²¹Sn, ¹²³I, ¹²⁴I, ¹²⁵I, ¹²⁷Cs, ¹²⁸Ba, ¹²⁹Cs, ¹³¹Cs, ¹³¹I, ¹³⁹La, ¹⁴⁰La, ¹⁴²Pr, ¹⁴³Pr, ¹⁴⁹Pm, ¹⁵¹Eu, ¹⁵³Eu, ¹⁵³Sm, ¹⁵⁹Gr, ¹⁶¹Tb, ¹⁶⁵Dy, ¹⁶⁶Ho, ¹⁶⁹Eu, ¹⁷⁵Yb, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁸Au, ¹⁹⁹Ag, ¹⁹⁹Au, ²⁰¹Tl, ²⁰³Pb, ²¹¹At, ²¹²Bi, ²¹²Pb, ²¹³Bi, ²²⁵Ac and ²²⁷Th.

In certain embodiments, the radioactive isotope is intended to enable imaging, such as by SPECT imaging and/or PET imaging. Single-photon emission computed tomography (SPECT) is a nuclear medicine tomographic imaging technique using gamma rays and is able to provide true 3D information. The information is often presented as cross-sectional slices through the patient. Due to the gamma-emission of the isotope, it is possible to see where the radiolabeled material has accumulated in the patient's body. Such a true 3D representation can be helpful in tumour imaging. Positron emission tomography (PET) is a nuclear medicine imaging technique that produces a 3D image and has a higher sensitivity than traditional SPECT imaging. The system detects pairs of gamma rays emitted indirectly by a positron-emitting radionuclide (tracer), which is introduced into the body. 3D images of tracer concentration within the body are then constructed by computer analysis and the 3D imaging is often accomplished with the aid of a computed tomography (CT) X-ray scan performed on the patient during the same session, in the same machine. Positron-emitting isotopes can also be used in conjunction with CT to provide 3D imaging of the anatomical distribution of a labelled medical device.

In certain embodiments, the radioactive isotope is an element in the group XIII (the Boron Family) of the periodic table, which includes Ga and In. In particular, preferred radioactive isotopes include Ga-67, Ga-68, Lu-177, Y-90, and In-111. Most preferably, radioactive isotopes are Lu-177 and Y-90. In one embodiment the radioactive isotope is Lu-177.

In certain embodiments, the radioactive isotope is a transition metals, such as Lu-177, Y-90, Cu-64, Cu-67 and Tb-161. Preferably, the radioactive isotope is Lu-177 or Y-90.

In certain embodiments, the ligand may include a combination of at least two radioactive isotopes to enable imaging and/or therapy. The combination of radioactive isotopes may be selected from Ga-68 and Lu-177; Ga-67 and Y-90; Ga-68 and Y-90; In-111 and Y-90; Lu-177 and Y-90, and Ga-67 and Tb-161.

The present invention may further include the use of at least one non-radioactive, non-toxic carrier metals. For example, the carrier metal may be selected from Bi and Fe. For instance, the non-radioactive carrier metal can be one which enables MRI imaging (for example Fe) or X-ray contrast imaging (for example Bi). Further examples of carrier metals include the trivalent bismuth, which additionally provides X-ray contrast in the microspheres, so that they can be imaged in CT.

In certain embodiments, the ligand includes a chelating moiety, e.g., a chelator for a radiometal or paramagnetic ion.

The chelating agent can comprise any chelator known in the art, see, e.g., Parus et al., “Chemistry and bifunctional chelating agents for binding (177)Lu,” Cuff Radiopharm. 2015; 8(2):86-94; Wangler et al., “Chelating agents and their use in radiopharmaceutical sciences,” Mini Rev Med Chem. 2011 October; 11(11):968-83; Liu, “Bifunctional Coupling Agents for Radiolabeling of Biomolecules and Target-Specific Delivery of Metallic Radionuclides,” Adv Drug Deliv Rev. 2008 September; 60(12): 1347-1370.

Illustrative examples include, for example:

| Chelator | Structure | R |
|---|---|---|
| DOTA | 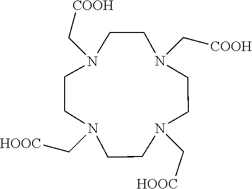 [SMILES: O=C(O)CN1CCN(CC(=O)O)CCN(CC(=O)O)CCN(CC(=O)O)CC1] | 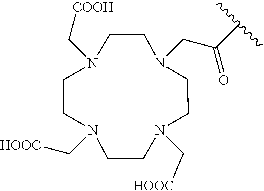 [SMILES: *C(=O)CN1CCN(CC(=O)O)CCN(CC(=O)O)CCN(CC(=O)O)CC1] |
| DOTA-NHS | 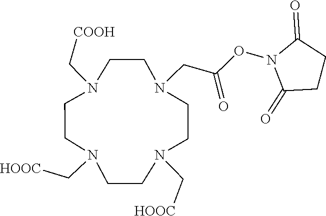 [SMILES: O=C(O)CN1CCN(CC(=O)O)CCN(CC(=O)ON2C(=O)CCC2=O)CCN(CC(=O)O)CC1] | 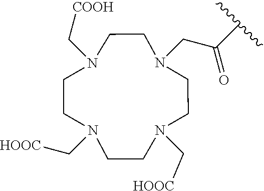 [SMILES: *C(=O)CN1CCN(CC(=O)O)CCN(CC(=O)O)CCN(CC(=O)O)CC1] |
| p-SCN-Bn-NOTA | 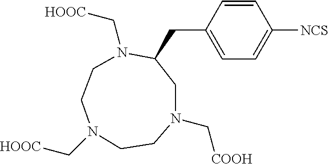 | 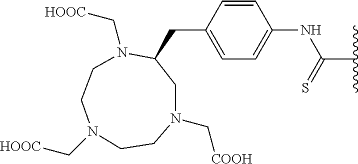 [SMILES: *C(=S)Nc1ccc(C[C@H]2CN(CC(=O)O)CCN(CC(=O)O)CCN2CC(=O)O)cc1] |
| p-SCN-Bn-PCTA | 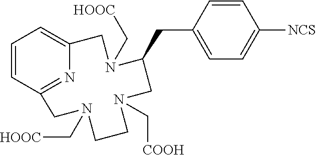 | 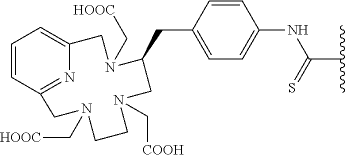 [SMILES: *C(=S)Nc1ccc(C[C@H]2CN(CC(=O)O)CCN(CC(=O)O)Cc3cccc(n3)CN2CC(=O)O)cc1] |
| p-SCN-Bn-Oxo- DO3A | 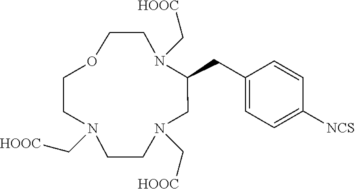 | 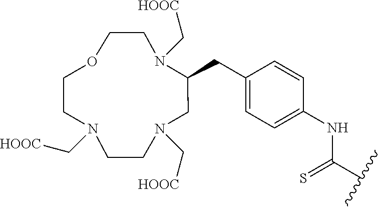 [SMILES: *C(=S)Nc1ccc(C[C@H]2CN(CC(=O)O)CCN(CC(=O)O)CCOCCN2CC(=O)O)cc1] |
| and desferrioxamine- p-SCN | 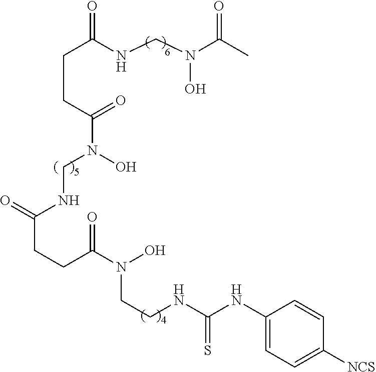 | 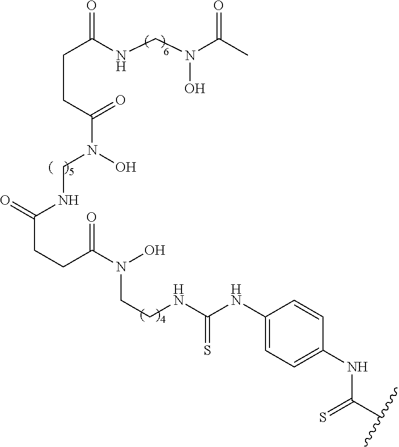 |
| Diethylenetriamin epentaacetic acid (DTPA) | 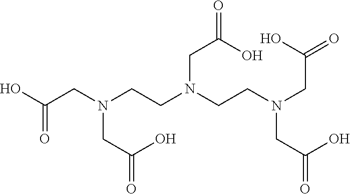 [SMILES: O=C(O)CN(CCN(CC(=O)O)CC(=O)O)CCN(CC(=O)O)CC(=O)O] | 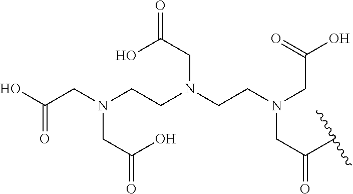 [SMILES: *C(=O)CN(CCN(CCN(CC(=O)O)CC(=O)O)CC(=O)O)CC(=O)O] |
| 1,4,8,11- tetraazacyclotetra decane1,4,8,11- tetraacetic acid (TETA) | 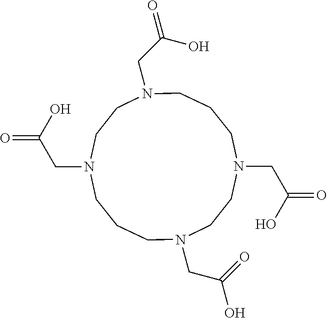 [SMILES: O=C(O)CN1CCCN(CC(=O)O)CCN(CC(=O)O)CCCN(CC(=O)O)CC1] | 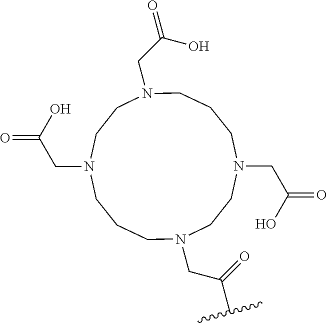 [SMILES: *C(=O)CN1CCCN(CC(=O)O)CCN(CC(=O)O)CCCN(CC(=O)O)CC1] |
| N,N′-Di(2- hydroxybenzyl) ethylenediamine- N,N′-diacetic acid (HBED) | 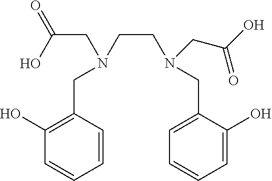 [SMILES: O=C(O)CN(CCN(CC(=O)O)Cc1ccccc1O)Cc1ccccc1O] | 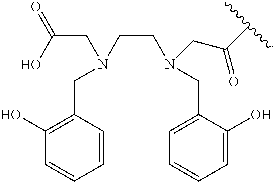 [SMILES: *C(=O)CN(CCN(CC(=O)O)Cc1ccccc1O)Cc1ccccc1O] |
| 4-(4,7-bis(2-(tert- butoxy)-2- oxoethyl)-1,4,7- triazacyclononan- 1-yl)-5-(tert- butoxy)-5- oxopentanoic acid (NODAG) | 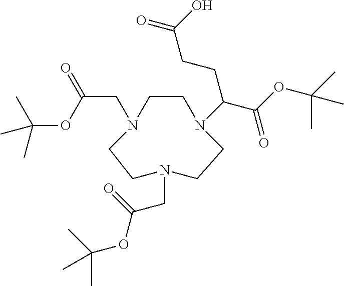 [SMILES: CC(C)(C)OC(=O)CN1CCN(CC(=O)OC(C)(C)C)CCN(C(CCC(=O)O)C(=O)OC(C)(C)C)CC1] | 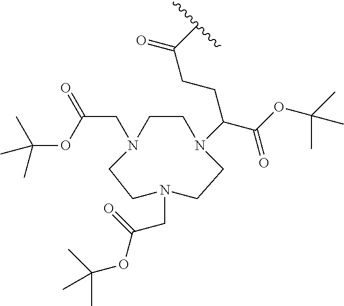 [SMILES: *C(=O)CCC(C(=O)OC(C)(C)C)N1CCN(CC(=O)OC(C)(C)C)CCN(CC(=O)OC(C)(C)C)CC1] |
| 2,2′-(1,4,8,11- tetraazabicyclo [6 [symbol] 6 [symbol] 2]hexadecane- 4,11-diyl)diacetic acid (CB-TE2A) | 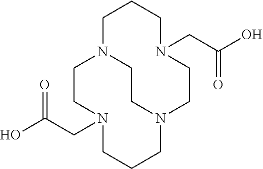 [SMILES: O=C(O)CN1CCCN2CCN(CCCN(CC(=O)O)CC2)CC1] | 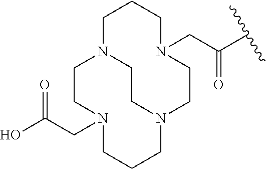 [SMILES: *C(=O)CN1CCCN2CCN(CCCN(CC(=O)O)CC2)CC1] |
| 6-amino-2-(11- (phosphonomethyl)- 1,4,8,11- tetraazabicyclo [6 [symbol] 6 [symbol] 2]hexadecan-4- yl)hexanoic acid (CB-TEIKIP) | 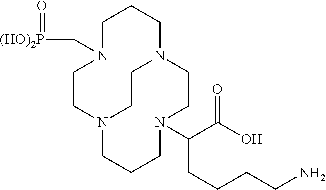 | 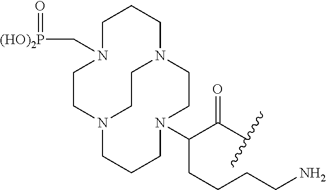 |
| HOPO | 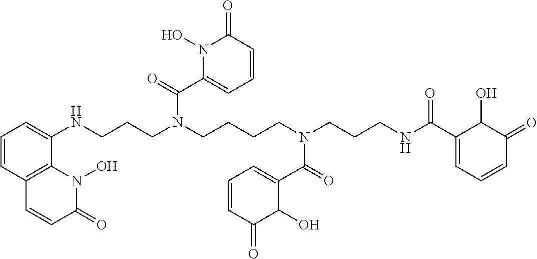 [SMILES: O=C(NCCCN(CCCCN(CCCNc1cccc2ccc(=O)n(O)c12)C(=O)c1cccc(=O)n1O)C(=O)C1=CC=CC(=O)C1O)C1=CC=CC(=O)C1O] | 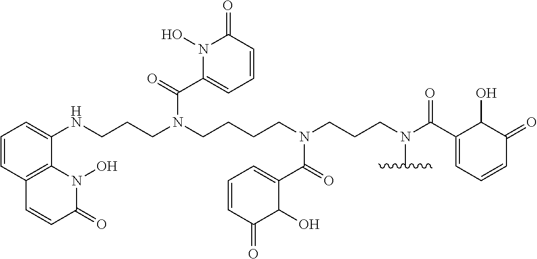 [SMILES: *N(CCCN(CCCCN(CCCNc1cccc2ccc(=O)n(O)c12)C(=O)c1cccc(=O)n1O)C(=O)C1=CC=CC(=O)C1O)C(=O)C1=CC=CC(=O)C1O] |
| DTPA | 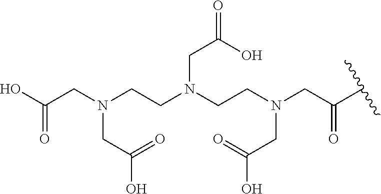 [SMILES: *C(=O)CN(CCN(CCN(CC(=O)O)CC(=O)O)CC(=O)O)CC(=O)O] | 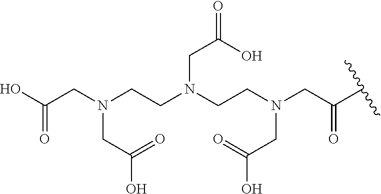 [SMILES: *C(=O)CN(CCN(CCN(CC(=O)O)CC(=O)O)CC(=O)O)CC(=O)O] |
| EDTA | 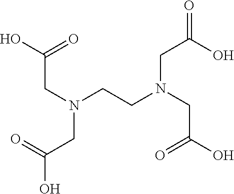 [SMILES: O=C(O)CN(CCN(CC(=O)O)CC(=O)O)CC(=O)O] | 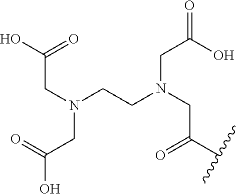 [SMILES: *C(=O)CN(CCN(CC(=O)O)CC(=O)O)CC(=O)O] |
| CHX-A [symbol] -DTPA | 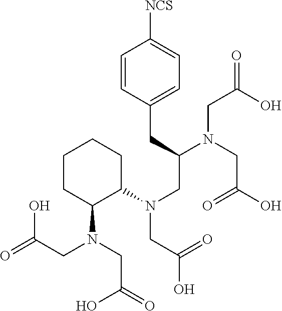 | 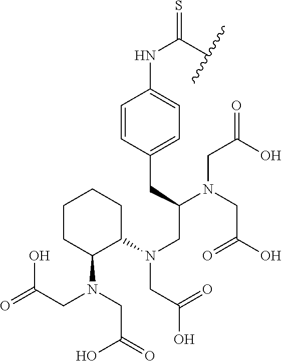 |
| NODASA | 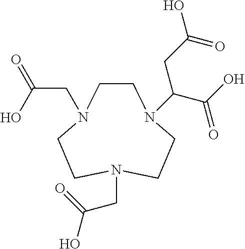 [SMILES: O=C(O)CC(C(=O)O)N1CCN(CC(=O)O)CCN(CC(=O)O)CC1] | 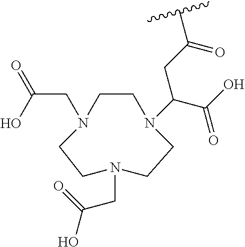 [SMILES: *C(=O)CC(C(=O)O)N1CCN(CC(=O)O)CCN(CC(=O)O)CC1] |
| TCMC | 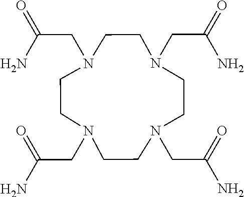 [SMILES: NC(=O)CN1CCN(CC(N)=O)CCN(CC(N)=O)CCN(CC(N)=O)CC1] | 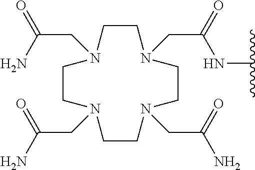 [SMILES: *NC(=O)CN1CCN(CC(N)=O)CCN(CC(N)=O)CCN(CC(N)=O)CC1] |
| TETA | 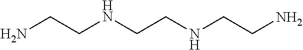 | 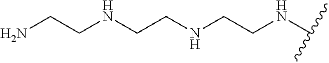 [SMILES: *NCCNCCNCCN] |
| PEPA | 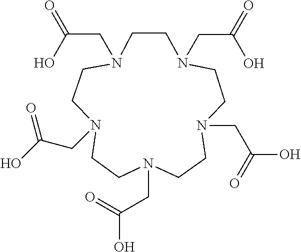 [SMILES: O=C(O)CN1CCN(CC(=O)O)CCN(CC(=O)O)CCN(CC(=O)O)CCN(CC(=O)O)CC1] | 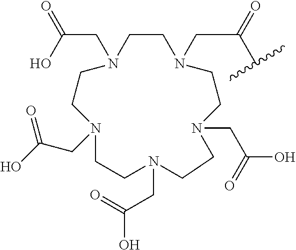 [SMILES: *C(=O)CN1CCN(CC(=O)O)CCN(CC(=O)O)CCN(CC(=O)O)CCN(CC(=O)O)CC1] |
| HEHA | 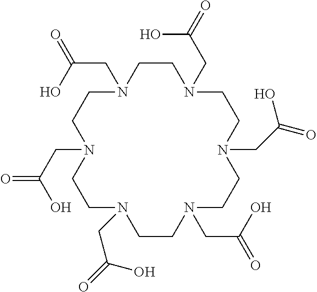 [SMILES: O=C(O)CN1CCN(CC(=O)O)CCN(CC(=O)O)CCN(CC(=O)O)CCN(CC(=O)O)CCN(CC(=O)O)CC1] | 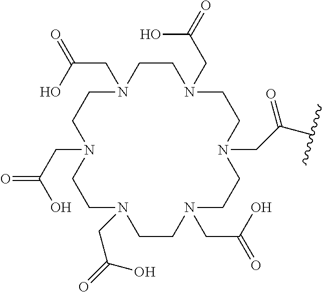 [SMILES: *C(=O)CN1CCN(CC(=O)O)CCN(CC(=O)O)CCN(CC(=O)O)CCN(CC(=O)O)CCN(CC(=O)O)CC1] |

[symbol]

indicates data missing or illegible when filed

Additional illustrative examples include, for example:

| Chelator | Structure | R |
|---|---|---|
| CB-DO2A | 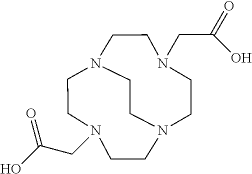 [SMILES: O=C(O)CN1CCN2CCN(CC1)CCN(CC(=O)O)CC2] | 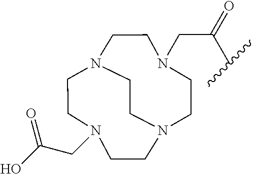 [SMILES: *C(=O)CN1CCN2CCN(CC1)CCN(CC(=O)O)CC2] |
| CB- TEAMA | 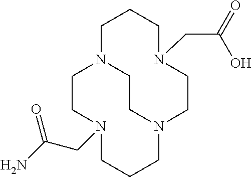 [SMILES: NC(=O)CN1CCCN2CCN(CCCN(CC(=O)O)CC2)CC1] | 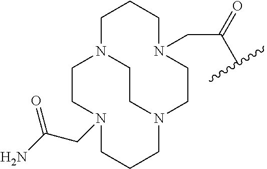 [SMILES: *C(=O)CN1CCCN2CCN(CCCN(CC(N)=O)CC2)CC1] |
| CB- MeTEAMA | 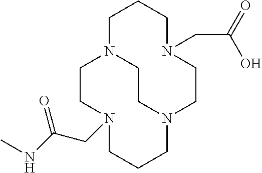 [SMILES: CNC(=O)CN1CCCN2CCN(CCCN(CC(=O)O)CC2)CC1] | 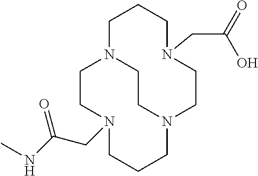 |
| CB- PhTEAMA | 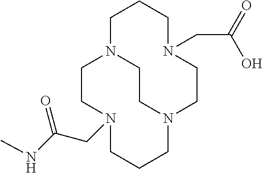 [SMILES: CNC(=O)CN1CCCN2CCN(CCCN(CC(=O)O)CC2)CC1] | 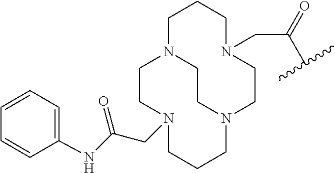 [SMILES: *C(=O)CN1CCCN2CCN(CCCN(CC(=O)Nc3ccccc3)CC2)CC1] |
| TATE | 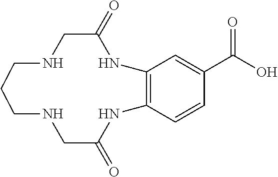 [SMILES: O=C1CNCCCNCC(=O)Nc2cc(C(=O)O)ccc2N1] | 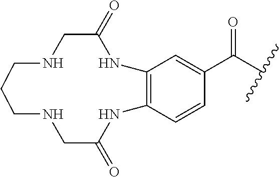 [SMILES: *C(=O)c1ccc2c(c1)NC(=O)CNCCCNCC(=O)N2] |

| | |
|---|---|
| Diamsar | |
| Diamsar | |
| H2ATSE/A | 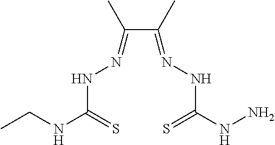 [SMILES: CCNC(=S)N/N=C(C)/C(C)=N/NC(=S)NN] |
| H2ATSE/A | 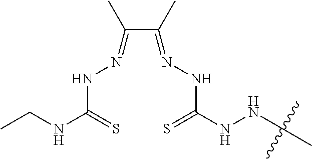 [SMILES: *NNC(=S)N/N=C(C)/C(C)=N/NC(=S)NCC] |

In certain preferred embodiments, the ligand can include DOTA, i.e., covalently linked to the ligand through any of its four carboxylic acid groups.

In certain embodiments, the chelator includes a radioactive isotope chelated therewith.

In certain embodiments, the chelator includes a paramagnetic ion chelated therewith. Examples of paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), erbium (III), or combinations of these paramagnetic ions.

Where the moiety is a detectable label, it can also be a fluorescent label. That is, in a certain embodiments, the ligand includes a fluorescent dye conjugated thereto, such as may be select from the group consisting of Xanthens, Acridines, Oxazines, Cynines, Styryl dyes, Coumarines, Porphines, Metal-Ligand-Complexes, Fluorescent proteins, Nanocrystals, Perylenes, Boron-dipyrromethenes and Phtalocyanines as well as conjugates and combinations of these classes of dyes. Examples of specific fluorescent labels include, but are not restricted to, organic dyes such as cyanine, fluorescein, rhodamine, Alexa Fluors, Dylight fluors, ATTO Dyes, BODIPY Dyes, etc. and biological fluorophores such as green fluorescent protein (GFP), R-Phycoerythrin, etc., and quantum dots.

In certain embodiments, the fluorescent moiety is selected from the group consisting of Cy5, Cy5.5 (also known as Cy5++), Cy2, fluorescein isothiocyanate (FITC), tetramethylrhodamine isothiocyanate (TRITC), phycoerythrin, Cy7, fluorescein (FAM), Cy3, Cy3.5 (also known as Cy3++), Texas Red, LightCycler-Red 640, LightCycler Red 705, tetramethylrhodamine (TMR), rhodamine, rhodamine derivative (ROX), hexachlorofluorescein (HEX), rhodamine 6G (R6G), the rhodamine derivative JA133, Alexa Fluorescent Dyes (such as Alexa Fluor 488, Alexa Fluor 546, Alexa Fluor 633, Alexa Fluor 555, and Alexa Fluor 647), 4′,6-diamidino-2-phenylindole (DAPI), Propidium iodide, AMCA, Spectrum Green, Spectrum Orange, Spectrum Aqua, Lissamine, and fluorescent transition metal complexes, such as europium. Fluorescent compound that can be used also include fluorescent proteins, such as GFP (green fluorescent protein), enhanced GFP (EGFP), blue fluorescent protein and derivatives (BFP, EBFP, EBFP2, Azurite, mKalama1), cyan fluorescent protein and derivatives (CFP, ECFP, Cerulean, CyPet) and yellow fluorescent protein and derivatives (YFP, Citrine, Venus, YPet). See also WO2008142571, WO2009056282, WO9922026.

### IV. Exemplary Therapeutic Uses of FAP-Activated Radiopharmaceuticals

And still another aspect of the invention provides methods for diagnosing, imaging or reducing tissue overexpressing FAP in an animal (preferably a human patient), comprising administering to the animal an FAP-activated theranostic prodrug of the present invention.

In some embodiments, the tissue overexpressing FAP is a tumor, especially a solid tumor. In some embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In some embodiments, the tumor is a colorectal tumor. In some embodiments, the tumor is an ovarian tumor. In some embodiments, the tumor is a lung tumor. In some embodiments, the tumor is a pancreatic tumor. In some embodiments, the tumor is a melanoma tumor. In some embodiments, the tumor is a bladder tumor.

To further illustrate, the subject FAP-activated radiopharmaceutical prodrugs can be used to treat patients suffering from cancer, such as osteosarcoma, rhabdomyosarcoma, neuroblastoma, kidney cancer, leukemia, renal transitional cell cancer, bladder cancer, Wilm's cancer, ovarian cancer, pancreatic cancer, breast cancer (including triple negative breast cancer), prostate cancer, bone cancer, lung cancer (e.g., small cell or non-small cell lung cancer), gastric cancer, colorectal cancer, cervical cancer, synovial sarcoma, head and neck cancer, squamous cell carcinoma, multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, a primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, carcinoid cancer or liver cancer, breast cancer or gastric cancer. In some embodiments of the disclosure, the cancer is metastatic cancer, e.g., of the varieties described above.

In some embodiments, in addition to administering an FAP-activated radiopharmaceutical prodrugs described herein, the method or treatment further comprises administering at least one additional immune response stimulating agent. In some embodiments, the additional immune response stimulating agent includes, but is not limited to, a colony stimulating factor (e.g., granulocyte-macrophage colony stimulating factor (GM-CSF), macrophage colony stimulating factor (M-CSF), granulocyte colony stimulating factor (G-CSF), stem cell factor (SCF)), an interleukin (e.g., IL-1, IL2, IL-3, IL-7, IL-12, IL-15, IL-18), a checkpoint inhibitor, an antibody that blocks immunosuppressive functions (e.g., an anti-CTLA-4 antibody, anti-CD28 antibody, anti-CD3 antibody), a toll-like receptor (e.g., TLR4, TLR7, TLR9), or a member of the B7 family (e.g., CD80, CD86). An additional immune response stimulating agent can be administered prior to, concurrently with, and/or subsequently to, administration of the FAP-activated radiopharmaceutical prodrug. Pharmaceutical compositions comprising an FAP-activated radiopharmaceutical prodrug and the immune response stimulating agent(s) are also provided. In some embodiments, the immune response stimulating agent comprises 1, 2, 3, or more immune response stimulating agents.

In some embodiments, in addition to administering an FAP-activated radiopharmaceutical prodrug described herein, the method or treatment further comprises administering at least one additional therapeutic agent. An additional therapeutic agent can be administered prior to, concurrently with, and/or subsequently to, administration of the FAP-activated radiopharmaceutical prodrug. Pharmaceutical compositions comprising an FAP-activated radiopharmaceutical prodrug and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the FAP-activated radiopharmaceutical prodrug. Combination therapy may decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some embodiments of the methods described herein, the combination of an FAP-activated radiopharmaceutical prodrug described herein and at least one additional therapeutic agent results in additive or synergistic results. In some embodiments, the combination therapy results in an increase in the therapeutic index of the FAP-activated radiopharmaceutical prodrug. In some embodiments, the combination therapy results in an increase in the therapeutic index of the additional therapeutic agent(s). In some embodiments, the combination therapy results in a decrease in the toxicity and/or side effects of the FAP-activated radiopharmaceutical prodrug. In some embodiments, the combination therapy results in a decrease in the toxicity and/or side effects of the additional therapeutic agent(s).

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, anti-metabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In some embodiments, the second therapeutic agent is an alkylating agent, an antimetabolite, an antimitotic, a topoisomerase inhibitor, or an angiogenesis inhibitor.

Therapeutic agents that may be administered in combination with the FAP-activated radiopharmaceutical prodrug described herein include chemotherapeutic agents. Thus, in some embodiments, the method or treatment involves the administration of an FAP-activated radiopharmaceutical prodrug of the present disclosure in combination with a chemotherapeutic agent or in combination with a cocktail of chemotherapeutic agents. Treatment with an FAP-activated radiopharmaceutical prodrug can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4.sup.th Edition, 2008, M. C. Perry, Editor, Lippincott, Williams & Wilkins, Philadelphia, Pa.

Chemotherapeutic agents useful in the present disclosure include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In some embodiments, the additional therapeutic agent is cisplatin. In some embodiments, the additional therapeutic agent is carboplatin.

In some embodiments of the methods described herein, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In some embodiments, the additional therapeutic agent is gemcitabine.

In some embodiments of the methods described herein, the chemotherapeutic agent is an antimitotic agent, including, but not limited to, agents that bind tubulin. In some embodiments, the agent is a taxane. In some embodiments, the agent is paclitaxel or docetaxel, or a pharmaceutically acceptable salt, acid, or derivative of paclitaxel or docetaxel. In some embodiments, the agent is paclitaxel (TAXOL), docetaxel (TAXOTERE), albumin-bound paclitaxel (nab-paclitaxel; ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In some embodiments, the additional therapeutic agent is paclitaxel. In some embodiments, the additional therapeutic agent is nab-paclitaxel.

In some embodiments of the methods described herein, an additional therapeutic agent comprises an agent such as a small molecule. For example, treatment can involve the combined administration of an FAP-activated radiopharmaceutical prodrug of the present disclosure with a small molecule that acts as an inhibitor against tumor-associated antigens including, but not limited to, EGFR, HER2 (ErbB2), and/or VEGF. In some embodiments, an FAP-activated radiopharmaceutical prodrug of the present disclosure is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In some embodiments of the methods described herein, the additional therapeutic agent is a small molecule that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an inhibitor of the Hippo pathway. In some embodiments, the additional therapeutic agent is an inhibitor of the mTOR/AKR pathway. In some embodiments, the additional therapeutic agent is an inhibitor of the RSPO/LGR pathway.

In some embodiments of the methods described herein, an additional therapeutic agent comprises a biological molecule, such as an antibody. For example, treatment can involve the combined administration of an FAP-activated radiopharmaceutical prodrug of the present disclosure with antibodies against tumor-associated antigens including, but not limited to, antibodies that bind EGFR, HER2/ErbB2, and/or VEGF. In some embodiments, the additional therapeutic agent is an antibody specific for a cancer stem cell marker. In some embodiments, the additional therapeutic agent is an antibody that binds a component of the Notch pathway. In some embodiments, the additional therapeutic agent is an antibody that binds a component of the Wnt pathway. In some embodiments, the additional therapeutic agent is an antibody that inhibits a cancer stem cell pathway. In some embodiments, the additional therapeutic agent is an inhibitor of the Notch pathway. In some embodiments, the additional therapeutic agent is an inhibitor of the Wnt pathway. In some embodiments, the additional therapeutic agent is an inhibitor of the BMP pathway. In some embodiments, the additional therapeutic agent is an antibody that inhibits .beta.-catenin signaling. In some embodiments, the additional therapeutic agent is an antibody that is an angiogenesis inhibitor (e.g., an anti-VEGF or VEGF receptor antibody). In some embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

In some embodiments of the methods described herein, the additional therapeutic agent is an antibody that modulates the immune response. In some embodiments, the additional therapeutic agent is an anti-PD-1 antibody, an anti-LAG-3 antibody, an anti-CTLA-4 antibody, an anti-TIM-3 antibody, or an anti-TIGIT antibody.

Furthermore, treatment with an FAP-activated radiopharmaceutical prodrug described herein can include combination treatment with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician. In some embodiments, the additional therapeutic agent is an immune response stimulating agent.

In some embodiments of the methods described herein, the FAP-activated radiopharmaceutical prodrug can be combined with a growth factor selected from the group consisting of: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-□, TNF-α, VEGF, P1GF, IL-1, IL-2, IL-3, IL-4, IL-5, IL-6, IL-7, IL-12, IL-15, and IL-18.

In some embodiments of the methods described herein, the additional therapeutic agent is an immune response stimulating agent. In some embodiments, the immune response stimulating agent is selected from the group consisting of granulocyte-macrophage colony stimulating factor (GM-CSF), macrophage colony stimulating factor (M-CSF), granulocyte colony stimulating factor (G-CSF), interleukin 3 (IL-3), interleukin 12 (IL-12), interleukin 1 (IL-1), interleukin 2 (IL-2), B7-1 (CD80), B7-2 (CD86), 4-1BB ligand, anti-CD3 antibody, anti-CTLA-4 antibody, anti-TIGIT antibody, anti-PD-1 antibody, anti-LAG-3 antibody, and anti-TIM-3 antibody.

In some embodiments of the methods described herein, an immune response stimulating agent is selected from the group consisting of: a modulator of PD-1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

In some embodiments of the methods described herein, an immune response stimulating agent is selected from the group consisting of: a PD-1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, and/or an IDO1 antagonist.

In some embodiments of the methods described herein, the PD-1 antagonist is an antibody that specifically binds PD-1. In some embodiments, the antibody that binds PD-1 is KEYTRUDA (MK-3475), pidilizumab (CT-011), nivolumab (OPDIVO, BMS-936558, MDX-1106), MEDI0680 (AMP-514), REGN2810, BGB-A317, PDR-001, or STI-A1110. In some embodiments, the antibody that binds PD-1 is described in PCT Publication WO 2014/179664, for example, an antibody identified as APE2058, APE1922, APE1923, APE1924, APE 1950, or APE1963, or an antibody containing the CDR regions of any of these antibodies. In other embodiments, the PD-1 antagonist is a fusion protein that includes PD-L2, for example, AMP-224. In other embodiments, the PD-1 antagonist is a peptide inhibitor, for example, AUNP-12.

In some embodiments, the CTLA-4 antagonist is an antibody that specifically binds CTLA-4. In some embodiments, the antibody that binds CTLA-4 is ipilimumab (YERVOY) or tremelimumab (CP-675,206). In some embodiments, the CTLA-4 antagonist a CTLA-4 fusion protein, for example, KAHR-102.

In some embodiments, the LAG3 antagonist is an antibody that specifically binds LAG3. In some embodiments, the antibody that binds LAG3 is IMP701, IMP731, BMS-986016, LAG525, and GSK2831781. In some embodiments, the LAG3 antagonist includes a soluble LAG3 receptor, for example, IMP321.

In some embodiments, the KIR antagonist is an antibody that specifically binds KIR. In some embodiments, the antibody that binds KIR is lirilumab.

In some embodiments, an immune response stimulating agent is selected from the group consisting of: a CD28 agonist, a 4-1BB agonist, an OX40 agonist, a CD27 agonist, a CD80 agonist, a CD86 agonist, a CD40 agonist, and a GITR agonist. p In some embodiments, the OX40 agonist includes OX40 ligand, or an OX40-binding portion thereof. For example, the OX40 agonist may be MEDI6383. In some embodiments, the OX40 agonist is an antibody that specifically binds OX40. In some embodiments, the antibody that binds OX40 is MEDI6469, MEDI0562, or MOXR0916 (RG7888). In some embodiments, the OX40 agonist is a vector (e.g., an expression vector or virus, such as an adenovirus) capable of expressing OX40 ligand. In some embodiments the OX40-expressing vector is Delta-24-RGDOX or DNX2401.

In some embodiments, the 4-1BB (CD137) agonist is a binding molecule, such as an anticalin. In some embodiments, the anticalin is PRS-343. In some embodiments, the 4-1BB agonist is an antibody that specifically binds 4-1BB. In some embodiments, antibody that binds 4-1BB is PF-2566 (PF-05082566) or urelumab (BMS-663513).

In some embodiments, the CD27 agonist is an antibody that specifically binds CD27. In some embodiments, the antibody that binds CD27 is varlilumab (CDX-1127).

In some embodiments, the GITR agonist comprises GITR ligand or a GITR-binding portion thereof. In some embodiments, the GITR agonist is an antibody that specifically binds GITR. In some embodiments, the antibody that binds GITR is TRX518, MK-4166, or INBRX-110.

In some embodiments, immune response stimulating agents include, but are not limited to, cytokines such as chemokines, interferons, interleukins, lymphokines, and members of the tumor necrosis factor (TNF) family. In some embodiments, immune response stimulating agents include immunostimulatory oligonucleotides, such as CpG dinucleotides.

In some embodiments, an immune response stimulating agent includes, but is not limited to, anti-PD-1 antibodies, anti-PD-L2 antibodies, anti-CTLA-4 antibodies, anti-CD28 antibodies, anti-CD80 antibodies, anti-CD86 antibodies, anti-4-1BB antibodies, anti-OX40 antibodies, anti-KIR antibodies, anti-Tim-3 antibodies, anti-LAG3 antibodies, anti-CD27 antibodies, anti-CD40 antibodies, anti-GITR antibodies, anti-TIGIT antibodies, anti-CD20 antibodies, anti-CD96 antibodies, or anti-IDO1 antibodies.

In some embodiments, the FAP-activated radiopharmaceutical prodrugs disclosed herein may be used alone, or in association with radiation therapy.

In some embodiments, the FAP-activated radiopharmaceutical prodrugs disclosed herein may be used alone, or in association with targeted therapies. Examples of targeted therapies include: hormone therapies, signal transduction inhibitors (e.g., EGFR inhibitors, such as cetuximab (Erbitux) and erlotinib (Tarceva)); HER2 inhibitors (e.g., trastuzumab (Herceptin) and pertuzumab (Perjeta)); BCR-ABL inhibitors (such as imatinib (Gleevec) and dasatinib (Sprycel)); ALK inhibitors (such as crizotinib (Xalkori) and ceritinib (Zykadia)); BRAF inhibitors (such as vemurafenib (Zelboraf) and dabrafenib (Tafinlar)), gene expression modulators, apoptosis inducers (e.g., bortezomib (Velcade) and carfilzomib (Kyprolis)), angiogenesis inhibitors (e.g., bevacizumab (Avastin) and ramucirumab (Cyramza), monoclonal antibodies attached to toxins (e.g., brentuximab vedotin (Adcetris) and ado-trastuzumab emtansine (Kadcyla)).

In some embodiments of the disclosure, an FAP-activated radiopharmaceutical prodrug of the disclosure is administered in association with a STING agonist, for example, as part of a pharmaceutical composition. The cyclic-di-nucleotides (CDNs) cyclic-di-AMP (produced by Listeria monocytogenes and other bacteria) and its analogs cyclic-di-GMP and cyclic-GMP-AMP are recognized by the host cell as a pathogen associated molecular pattern (PAMP), which bind to the pathogen recognition receptor (PRR) known as Stimulator of INterferon Genes (STING). STING is an adaptor protein in the cytoplasm of host mammalian cells which activates the TANK binding kinase (TBK1)—IRF3 and the NF-.kappa.B signaling axis, resulting in the induction of IFN-.beta. and other gene products that strongly activate innate immunity. It is now recognized that STING is a component of the host cytosolic surveillance pathway, that senses infection with intracellular pathogens and in response induces the production of IFN-α, leading to the development of an adaptive protective pathogen-specific immune response consisting of both antigen-specific CD4+ and CD8+ T cells as well as pathogen-specific antibodies. U.S. Pat. Nos. 7,709,458 and 7,592,326; PCT Publication Nos. WO2007/054279, WO2014/093936, WO2014/179335, WO2014/189805, WO2015/185565, WO2016/096174, WO2016/145102, WO2017/027645, WO2017/027646, and WO2017/075477; and Yan et al., Bioorg. Med. Chem Lett. 18:5631-4, 2008.

In some embodiments of the disclosure, an FAP-activated radiopharmaceutical prodrug of the disclosure is administered in association with an Akt inhibitor. Exemplary AKT inhibitors include GDC0068 (also known as GDC-0068, ipatasertib and RG7440), MK-2206, perifosine (also known as KRX-0401), GSK690693, AT7867, triciribine, CCT128930, A-674563, PHT-427, Akti-1/2, afuresertib (also known as GSK2110183), AT13148, GSK2141795, BAY1125976, uprosertib (aka GSK2141795), Akt Inhibitor VIII (1,3-dihydro-1-[1-[[4-(6-phenyl-1H-imidazo[4,5-g]quinoxalin-7-yl)phenyl]m-ethyl]-4-piperidinyl]-2H-benzimidazol-2-one), Akt Inhibitor X (2-chloro-N,N-diethyl-10H-phenoxazine-10-butanamine, monohydrochloride), MK-2206 (8-(4-(1-aminocyclobutyl)phenyl)-9-phenyl-[1,2,4]triazolo[3,4-f][-1,6]naphthyridin-3(2H)-one), uprosertib (N—((S)-1-amino-3-(3,4-difluorophenyl)propan-2-yl)-5-chloro-4-(4-chloro-1-methyl-1H-pyrazol-5-yl)furan-2-carboxamide), ipatasertib ((S)-2-(4-chlorophenyl)-1-(4-((5R,7R)-7-hydroxy-5-methyl-6,7-dihydro-5H-c-yclopenta[d]pyrimidin-4-yl)piperazin-1-yl)-3-(isopropylamino)propan-1-one)-AZD 5363 (4-Piperidinecarboxamide, 4-amino-N-[(1S)-1-(4-chlorophenyl)-3-hydroxypropyl]-1-(7H-pyrrolo[2,3-d]p-yrimidin-4-yl)), perifosine, GSK690693, GDC-0068, tricirbine, CCT128930, A-674563, PF-04691502, AT7867, miltefosine, PHT-427, honokiol, triciribine phosphate, and KP372-1A (10H-indeno[2,1-e]tetrazolo[1,5-b][1,2,4]triazin-10-one), Akt Inhibitor IX (CAS 98510-80-6). Additional Akt inhibitors include: ATP-competitive inhibitors, e.g. isoquinoline-5-sulfonamides (e.g., H-8, H-89, NL-71-101), azepane derivatives (e.g., (−)-balanol derivatives), aminofurazans (e.g., GSK690693), heterocyclic rings (e.g., 7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363, A-674563, A-443654), phenylpyrazole derivatives (e.g., AT7867, AT13148), thiophenecarboxamide derivatives (e.g., Afuresertib (GSK2110183), 2-pyrimidyl-5-amidothiophene derivative (DC120), uprosertib (GSK2141795); Allosteric inhibitors, e.g., 2,3-diphenylquinoxaline analogues (e.g., 2,3-diphenylquinoxaline derivatives, triazolo[3,4-f][1,6]naphthyridin-3(2H)-one derivative (MK-2206)), alkylphospholipids (e.g., Edelfosine (1-O-octadecyl-2-O-methyl-rac-glycero-3-phosphocholine, ET-18-OCH3) ilmofosine (BM 41.440), miltefosine (hexadecylphosphocholine, HePC), perifosine (D-21266), erucylphosphocholine (ErPC), erufosine (ErPC3, erucylphosphohomocholine), indole-3-carbinol analogues (e.g., indole-3-carbinol, 3-chloroacetylindole, diindolylmethane, diethyl 6-methoxy-5,7-dihydroindolo [2,3-b]carbazole-2,10-dicarboxylate (SR13668), OSU-A9), Sulfonamide derivatives (e.g., PH-316, PHT-427), thiourea derivatives (e.g., PIT-1, PIT-2, DM-PIT-1, N-[(1-methyl-1H-pyrazol-4-yl)carbonyl]-N′-(3-bromophenyl)-thiourea), purine derivatives (e.g., Triciribine (TCN, NSC 154020), triciribine mono-phosphate active analogue (TCN-P),4-amino-pyrido[2,3-d]pyrimidine derivative API-1, 3-phenyl-3H-imidazo[4,5-b]pyridine derivatives, ARQ 092), BAY 1125976, 3-methyl-xanthine, quinoline-4-carboxamide, 2-[4-(cyclohexa-1,3-dien-1-yl)-1H-pyrazol-3-yl]phenol, 3-oxo-tirucallic acid, 3.alpha.- and 3.beta.-acetoxy-tirucallic acids, acetoxy-tirucallic acid; and irreversible inhibitors, e.g., natural products, antibiotics, Lactoquinomycin, Frenolicin B, kalafungin, medermycin, Boc-Phe-vinyl ketone, 4-hydroxynonenal (4-HNE), 1,6-naphthyridinone derivatives, and imidazo-1,2-pyridine derivatives.

In some embodiments of the disclosure, an FAP-activated radiopharmaceutical prodrug of the disclosure is administered in association with a MEK inhibitor. Exemplary MEK inhibitors include AZD6244 (Selumetinib), PD0325901, GSK1120212 (Trametinib), U0126-EtOH, PD184352, RDEA119 (Rafametinib), PD98059, BIX 02189, MEK162 (Binimetinib), AS-703026 (Pimasertib), SL-327, BIX02188, AZD8330, TAK-733, cobimetinib and PD318088.

In some embodiments of the disclosure, an FAP-activated radiopharmaceutical prodrug of the disclosure is administered in association with both an anthracycline such as doxorubicin and cyclophosphamide, including pegylated liposomal doxorubicin.

In some embodiments of the disclosure, an FAP-activated radiopharmaceutical prodrug of the disclosure is administered in association with both an anti-CD20 antibody and an anti-CD3 antibody, or a bispecific CD20/CD3 binder (including a CD20/CD3 BiTE).

In some embodiments of the disclosure, an FAP-activated radiopharmaceutical prodrug of the disclosure is administered in association with a CD73 inhibitor, a CD39 inhibitor or both. These inhibitors can be CD73 binders or CD39 binders (such as antibody, antibody fragments or antibody mimetics) that inhibit the ectonucleosidase activity. The inhibitor may be a small molecule inhibitor of the ectonucleosidase activity, such as 6-N,N-Diethyl-β-γ-dibromomethylene-D-adenosine-5′-triphosphate trisodium salt hydrate, PSB069, PSB 06126,

In some embodiments of the disclosure, an FAP-activated radiopharmaceutical prodrug of the disclosure is administered in association with an inhibitor poly ADP ribose polymerase (PARP). Exemplary PARP inhibitors include Olaparib, Niraparib, Rucaparib, Talazoparib, Veliparib, CEP9722, MK4827 and BGB-290.

In some embodiments of the disclosure, an FAP-activated radiopharmaceutical prodrug of the disclosure is administered in association with an oncolytic virus. An exemplary oncolytic virus is Talimogene Laherparepvec.

In some embodiments of the disclosure, an FAP-activated radiopharmaceutical prodrug of the disclosure is administered in association with an CSF-1 antagonist, such as an agent that binds to CSF-1 or CSF1R and inhibits the interaction of CSF-1 with CSF1R on macrophage. Exemplary CSF-1 antagonists include Emactuzumab and FPA008.

In some embodiments of the disclosure, an FAP-activated radiopharmaceutical prodrug of the disclosure is administered in association with an anti-CD38 antibody. Exemplary anti-CD39 antibodies include Daratumumab and Isatuximab.

In some embodiments of the disclosure, an FAP-activated radiopharmaceutical prodrug of the disclosure is administered in association with an anti-CD40 antibody. Exemplary anti-CD40 antibodies include Selicrelumab and Dacetuzumab.

In some embodiments of the disclosure, an FAP-activated radiopharmaceutical prodrug of the disclosure is administered in association with an inhibitor of anaplatic lymphoma kinase (ALK). Exemplary ALK inhibitors include Alectinib, Crizotinib and Ceritinib.

In some embodiments of the disclosure, an FAP-activated radiopharmaceutical prodrug of the disclosure is administered in association with multikinase inhibitor that inhibits one or more selected from the group consisting of the family members of VEGFR, PDGFR and FGFR, or an anti-angiogenesis inhibitor. Exemplary inhibitors include Axitinib, Cediranib, Linifanib, Motesanib, Nintedanib, Pazopanib, Ponatinib, Regorafenib, Sorafenib, Sunitinib, Tivozanib, Vatalanib, LY2874455, or SU5402.

In some embodiments of the disclosure, an FAP-activated radiopharmaceutical prodrug of the disclosure is administered in conjunction with one or more vaccines intended to stimulate an immune response to one or more predetermined antigens. The antigen(s) may be administered directly to the individual, or may be expressed within the individual from, for example, a tumor cell vaccine (e.g., GVAX) which may be autologous or allogenic, a dendritic cell vaccine, a DNA vaccine, an RNA vaccine, a viral-based vaccine, a bacterial or yeast vaccine (e.g., a Listeria monocytogenes or Saccharomyces cerevisiae), etc. See, e.g., Guo et al., Adv. Cancer Res. 2013; 119: 421-475; Obeid et al., Semin Oncol. 2015 August; 42(4): 549-561. The target antigen may also be a fragment or fusion polypeptide comprising an immunologically active portion of the antigens listed in the table.

In some embodiments of the disclosure, an FAP-activated radiopharmaceutical prodrug of the disclosure is administered in association with one or more antiemetics including, but not limited to: casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl by Pfizer; New York, N.Y.), hydroxyzine (sold as Atarax by Pfizer; New York, N.Y.), metoclopramide (sold as Reglan by AH Robins Co.; Richmond, Va.), lorazepam (sold as Ativan by Wyeth; Madison, N.J.), alprazolam (sold as Xanax by Pfizer; New York, N.Y.), haloperidol (sold as Haldol by Ortho-McNeil; Raritan, N.J.), droperidol (Inapsine), dronabinol (sold as Marinol by Solvay Pharmaceuticals, Inc.; Marietta, Ga.), dexamethasone (sold as Decadron by Merck and Co.; Rahway, N.J.), methylprednisolone (sold as Medrol by Pfizer; New York, N.Y.), prochlorperazine (sold as Compazine by Glaxosmithkline; Research Triangle Park, N.C.), granisetron (sold as Kytril by Hoffmann-La Roche Inc.; Nutley, N.J.), ondansetron (sold as Zofran by Glaxosmithkline; Research Triangle Park, N.C.), dolasetron (sold as Anzemet by Sanofi-Aventis; New York, N.Y.), tropisetron (sold as Navoban by Novartis; East Hanover, N.J.).

Other side effects of cancer treatment include red and white blood cell deficiency. Accordingly, in some embodiments of the disclosure, an FAP-activated radiopharmaceutical prodrug is administered in association with an agent which treats or prevents such a deficiency, such as, e.g., filgrastim, PEG-filgrastim, erythropoietin, epoetin alfa or darbepoetin alfa.

In some embodiments of the disclosure, an FAP-activated radiopharmaceutical prodrug of the disclosure is administered in association with anti-cancer radiation therapy. For example, in some embodiments of the disclosure, the radiation therapy is external beam therapy (EBT): a method for delivering a beam of high-energy X-rays to the location of the tumor. The beam is generated outside the patient (e.g., by a linear accelerator) and is targeted at the tumor site. These X-rays can destroy the cancer cells and careful treatment planning allows the surrounding normal tissues to be spared. No radioactive sources are placed inside the patient's body. In some embodiments of the disclosure, the radiation therapy is proton beam therapy: a type of conformal therapy that bombards the diseased tissue with protons instead of X-rays. In some embodiments of the disclosure, the radiation therapy is conformal external beam radiation therapy: a procedure that uses advanced technology to tailor the radiation therapy to an individual's body structures. In some embodiments of the disclosure, the radiation therapy is brachytherapy: the temporary placement of radioactive materials within the body, usually employed to give an extra dose—or boost—of radiation to an area.

### V. Exemplification

### Example 1

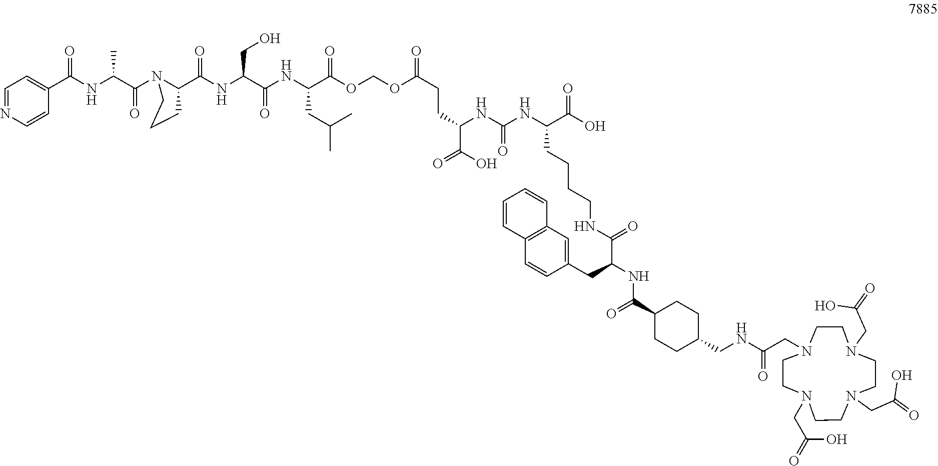

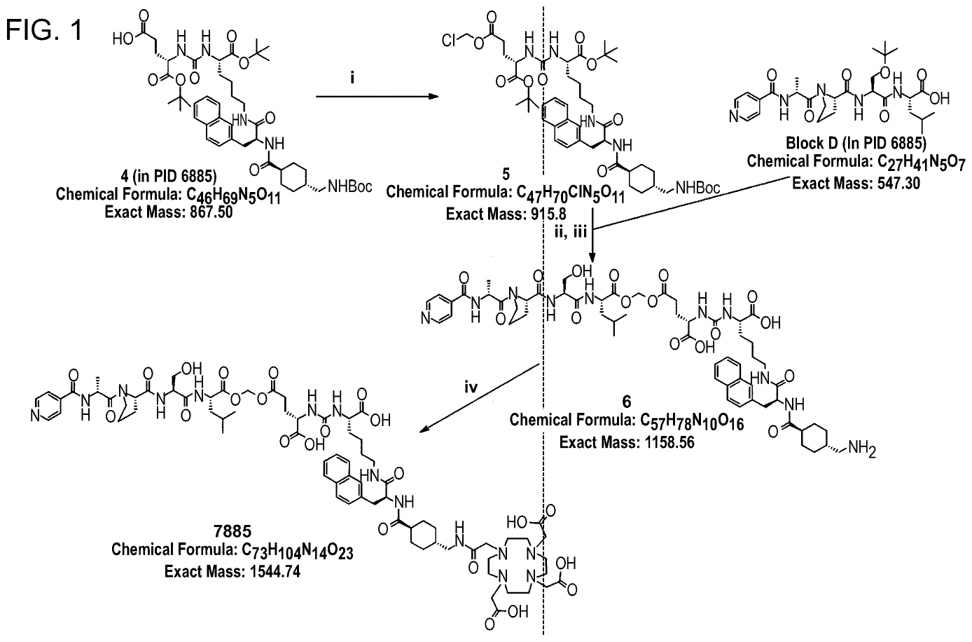

A synthetic scheme for the preparation of comound 7885 is depicted in FIG. 1, in which i. BrCH2Cl, NaHCO3; ii. TEA, NaI; iii. TFA-DCM; iv. DOTA-PNP.

### Example 2

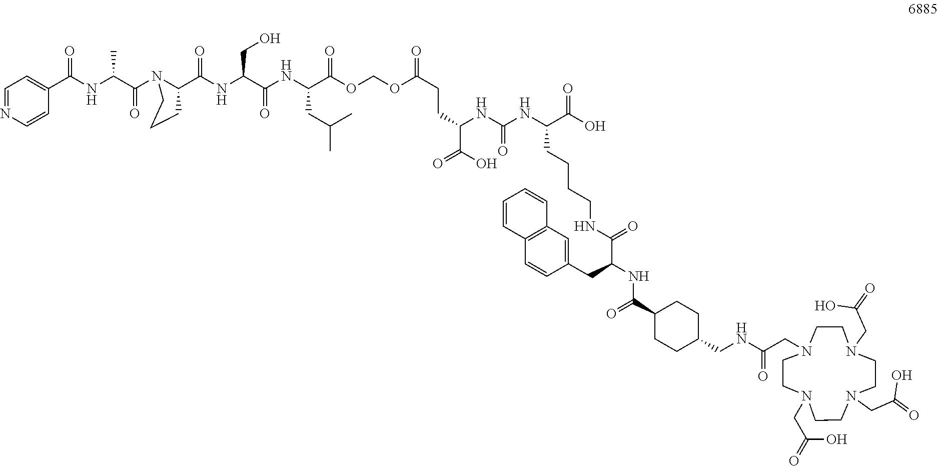

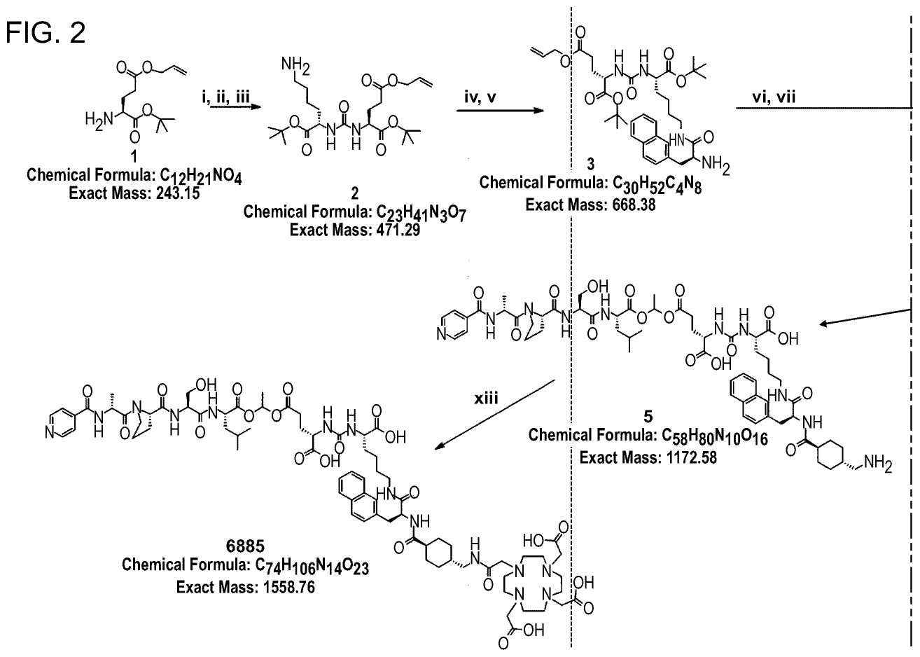

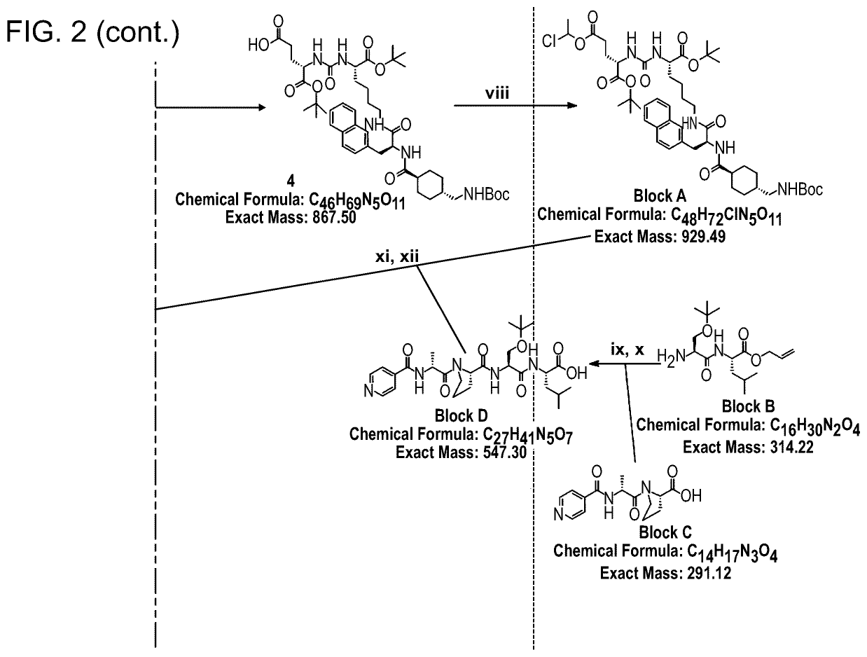

A synthetic scheme for the preparation of compound 6885 is depicted in FIG. 2 in which i. triphosgene, Py; ii. Lys(Fmoc)-OtBu, DIEA, Flash column purification; iii. 50% of TEA in DCM; iv. Fmoc-L-2-Nal-OH, HATU, DIEA, DMF, Flash column purification; v. 50% of TEA in DCM; vi. N-Boc-tranexamic acid, HBTU, DIEA, DMF, Flash column purification; vii. Pd(PPh3)4, Flash column purification; viii. BrClCHCH3, Cs2CO3; ix. HATU, DIEA; x. Pd(PPh3)4; xi. Cs2CO3; xii. TFA-DCM; xiii. DOTA-PNP.

### Example 3

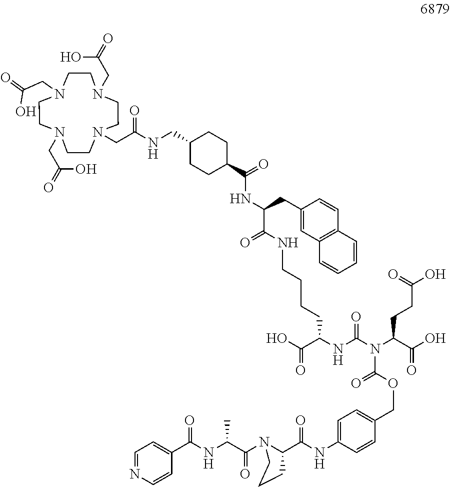

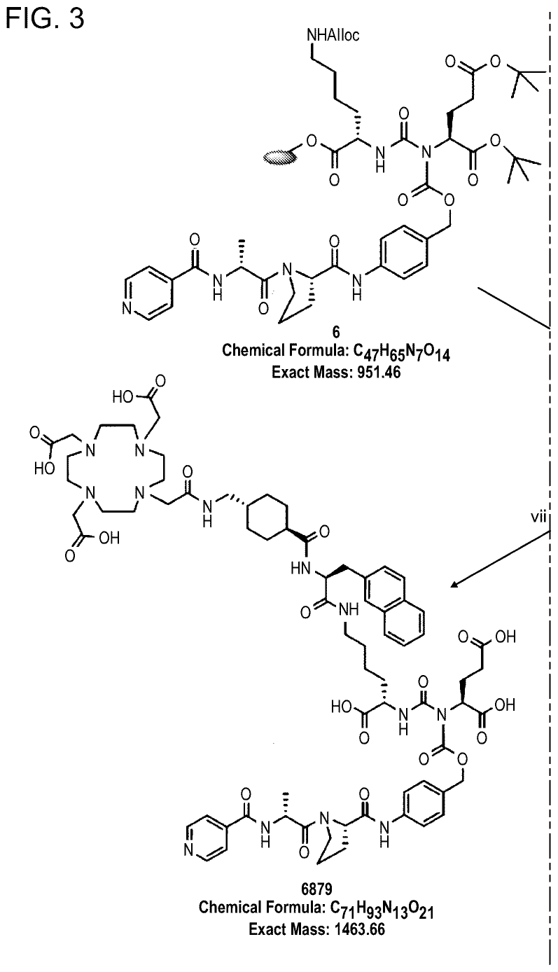

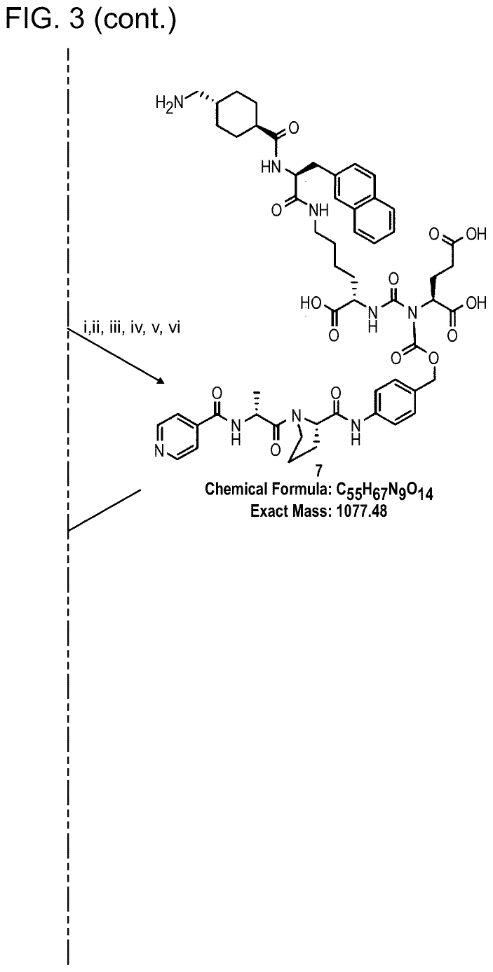

A synthetic scheme for compound 6879 is depicted in FIG. 3 in which i. Pd[PPh3]4, morpholine, DCM; ii. Fmoc-L-2-Nal-OH, HBTU, DIEA, DMF; iii. 50% piperidine in DMF; iv. N-Fmoc-tranexamic acid, HBTU, DIEA, DMF; v. 50% piperidine in DMF; vi. TFA, TIPS; vii. DOTA-PNP.

### Example 4

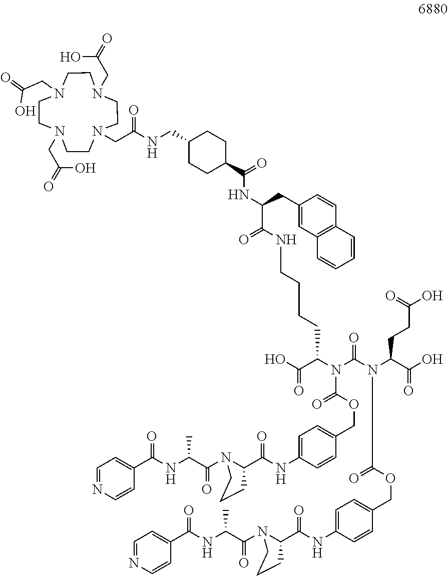

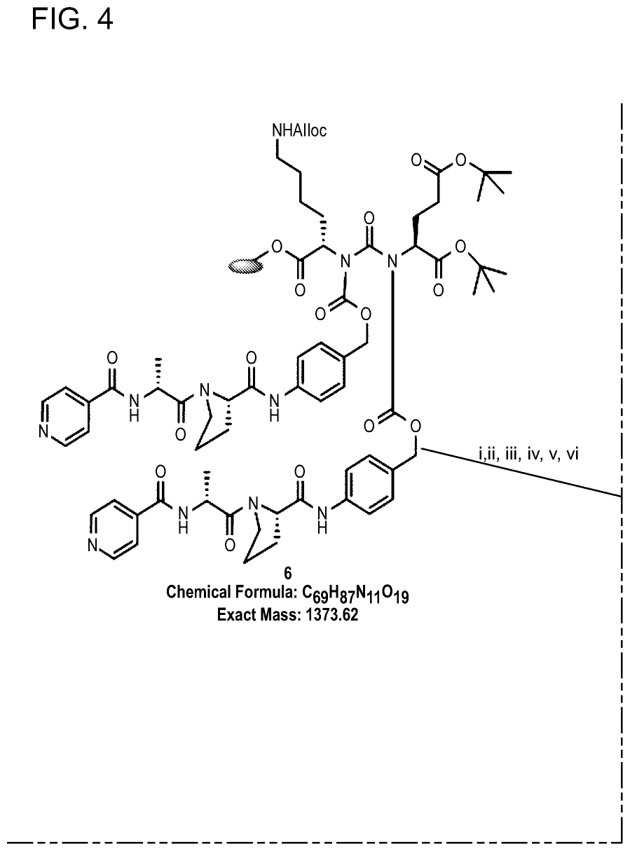

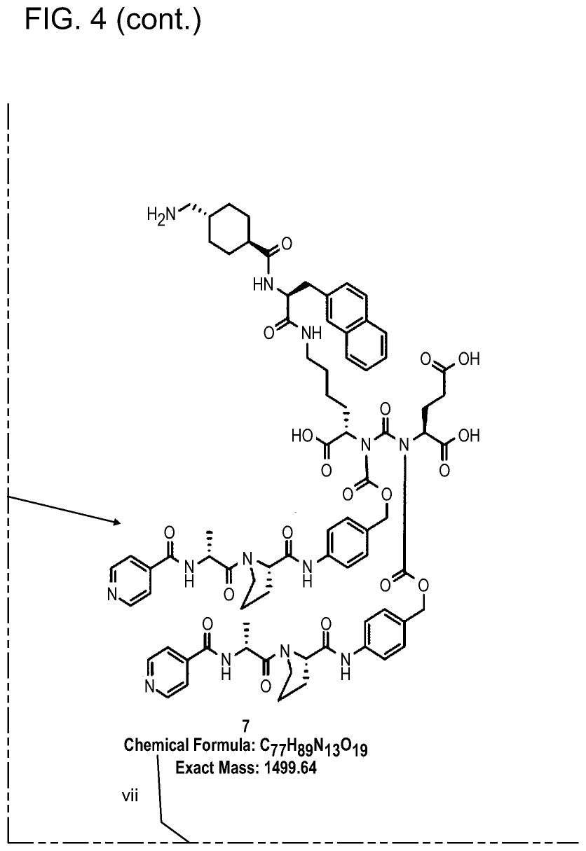

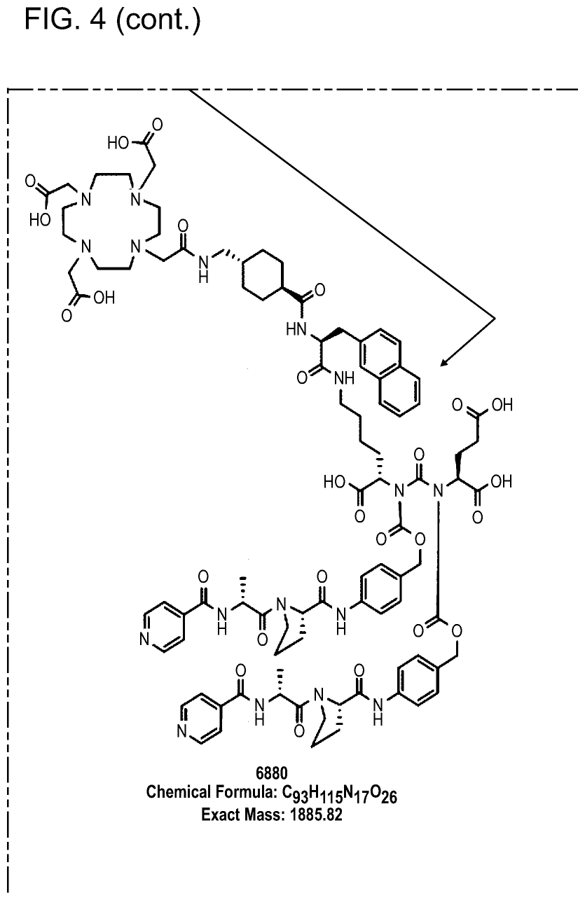

A synthetic scheme for compound 6880 is depicted in FIG. 4 in which i. Pd[PPh3]4, morpholine, DCM; ii. Fmoc-L-2-Nal-OH, HBTU, DIEA, DMF; iii. 50% piperidine in DMF; iv. N-Fmoc-tranexamic acid, HBTU, DIEA, DMF; v. 50% piperidine in DMF; vi. TFA, TIPS; vii. DOTA-PNP.

### Example 5

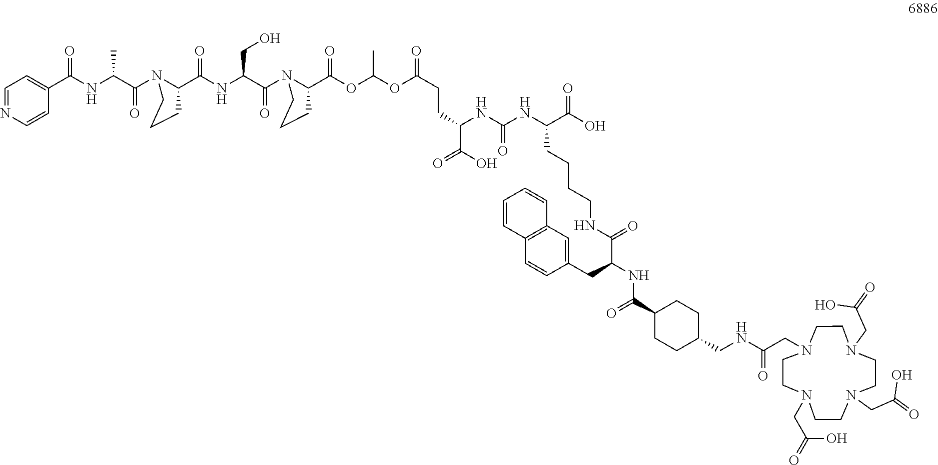

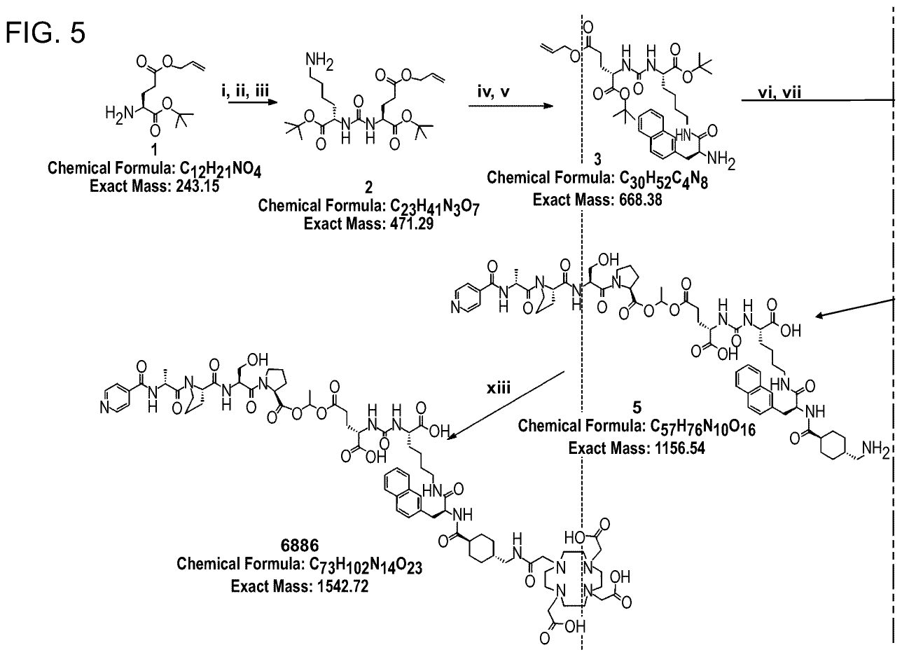

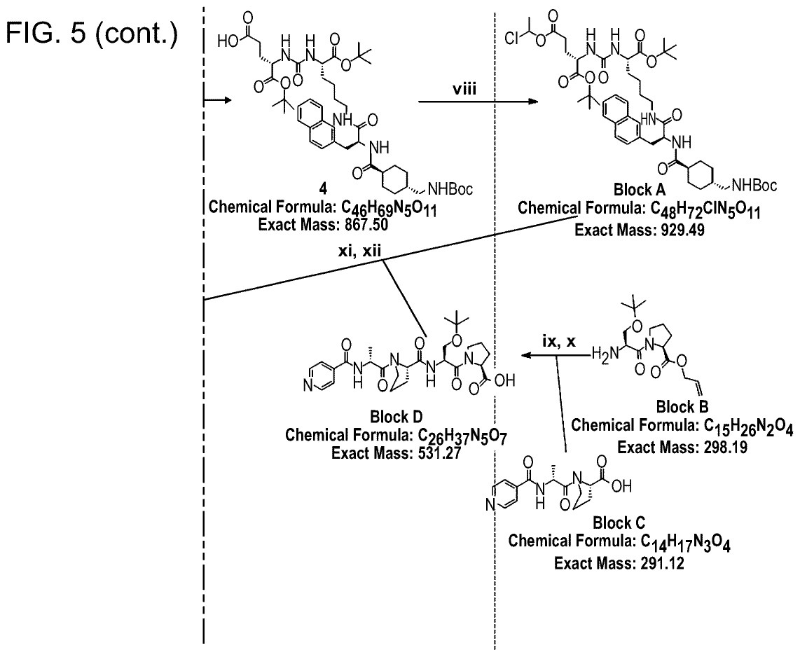

A synthetic scheme for compound 6886 is depicted in FIG. 5 in which i. triphosgene, Py; ii. Lys(Fmoc)-OtBu, DIEA; iii. 50% piperidine in DMF; iv. Fmoc-L-2-Nal-OH, HBTU, DIEA, DMF; v. 50% piperidine in DMF; vi. N-Boc-tranexamic acid, HBTU, DIEA, DMF; vii. Pd(PPh3)4; viii. BrClCH2, Cs2CO3; ix. HATU, DIEA; x. Pd(PPh3)4; xi. Cs2CO3; xii. TFA-DCM; xiii. DOTA-PNP.

### Example 6: Additional Compounds and Syntheses

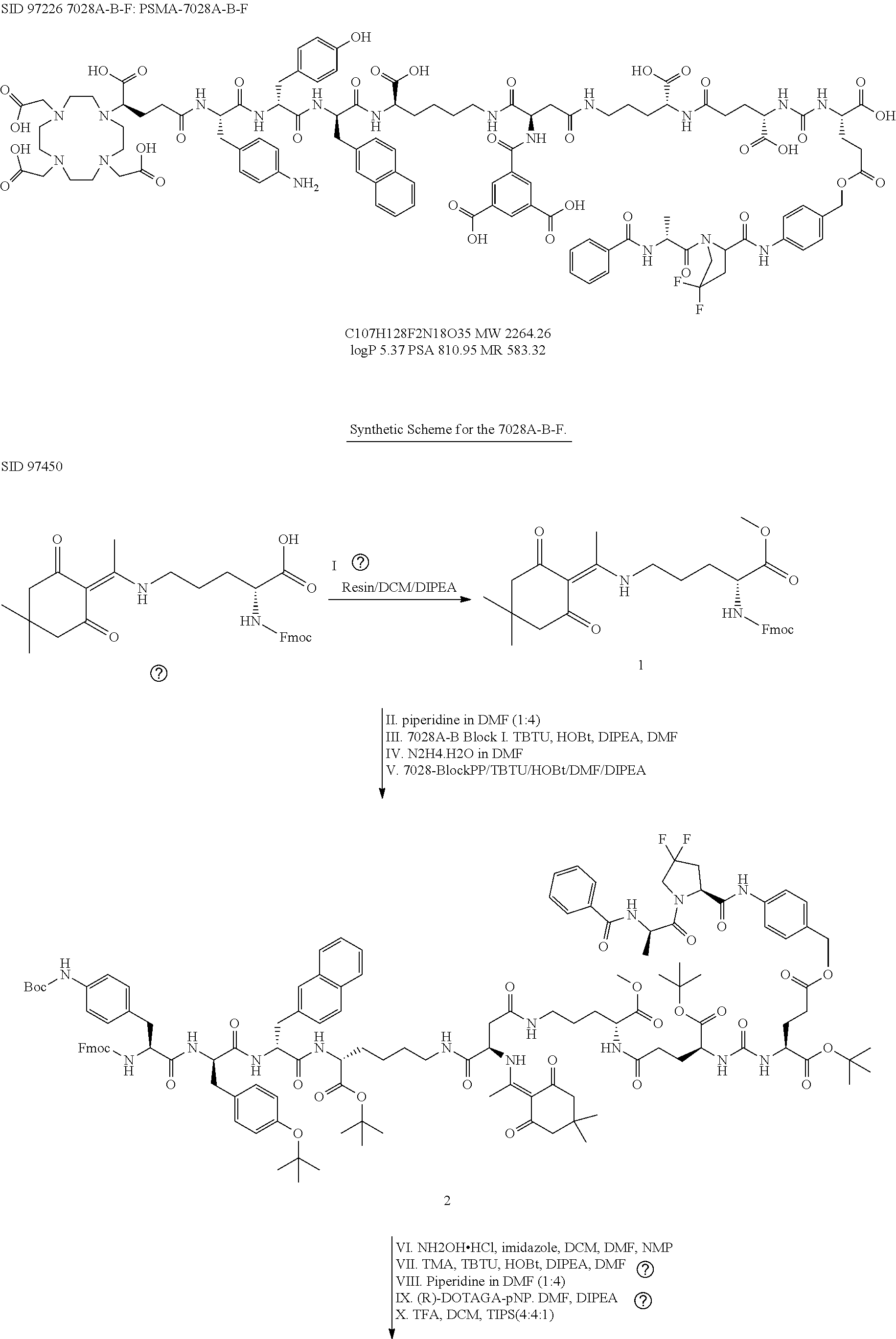

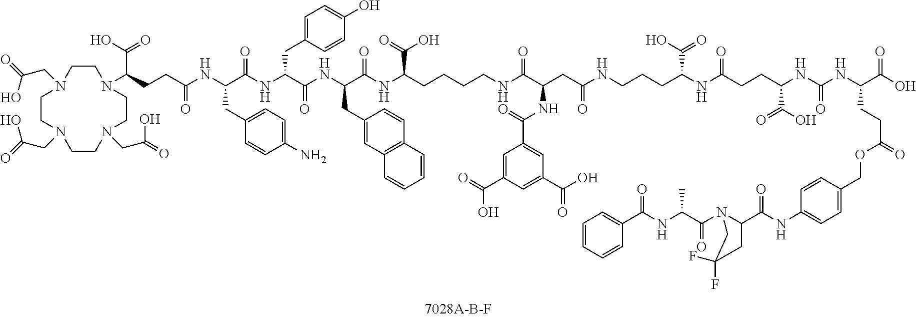 [SMILES: C[C@@H](NC(=O)c1ccccc1)C(=O)N1CC(F)(F)CC1C(=O)Nc1ccc(COC(=O)CC[C@H](NC(=O)N[C@@H](CCC(=O)N[C@H](CCCNC(=O)C[C@@H](NC(=O)c2cc(C(=O)O)cc(C(=O)O)c2)C(=O)NCCCC[C@@H](NC(=O)[C@@H](Cc2ccc3ccccc3c2)NC(=O)[C@@H](Cc2ccc(O)cc2)NC(=O)[C@H](Cc2ccc(N)cc2)NC(=O)CC[C@H](C(=O)O)N2CCN(CC(=O)O)CCN(CC(=O)O)CCN(CC(=O)O)CC2)C(=O)O)C(=O)O)C(=O)O)C(=O)O)cc1]

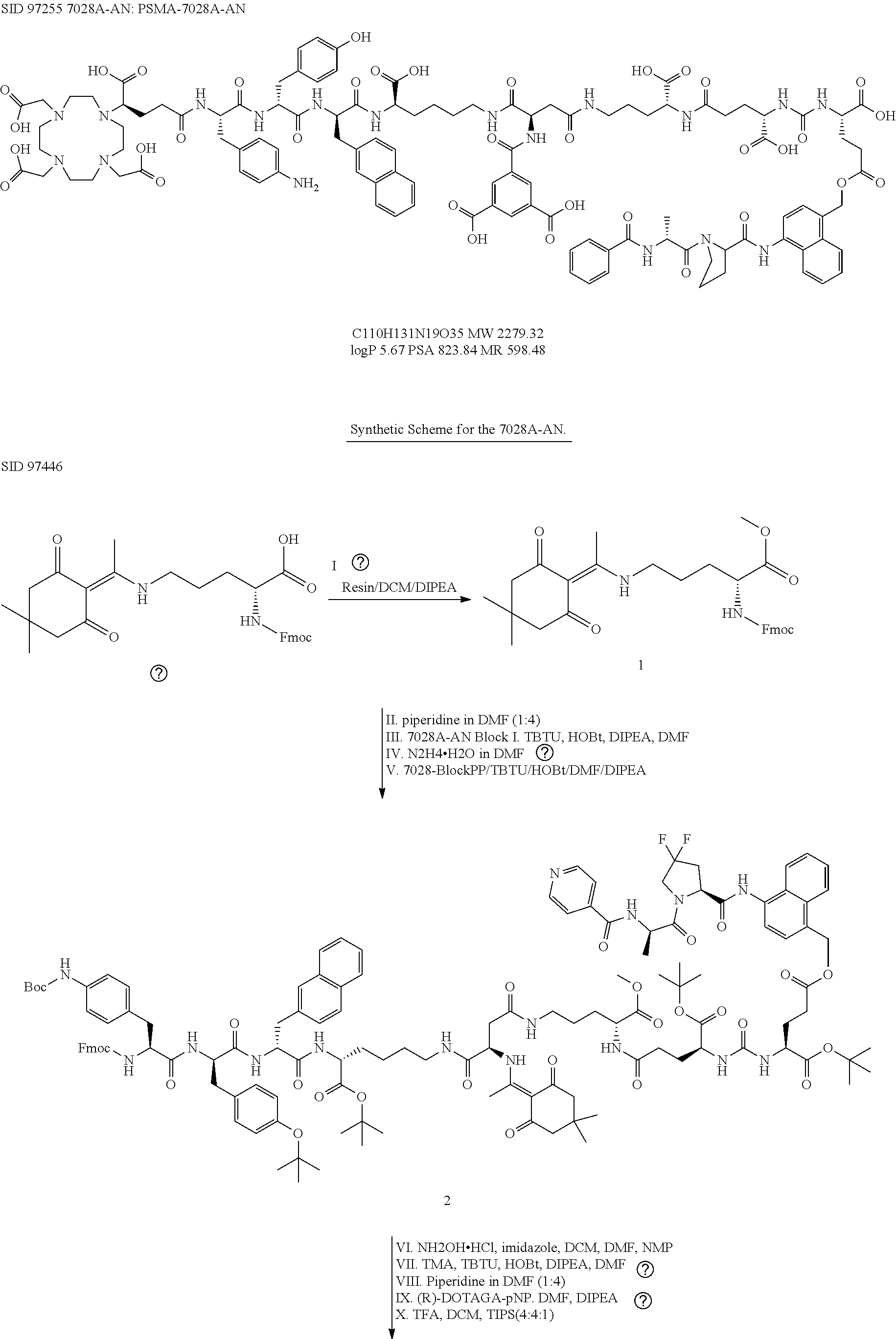

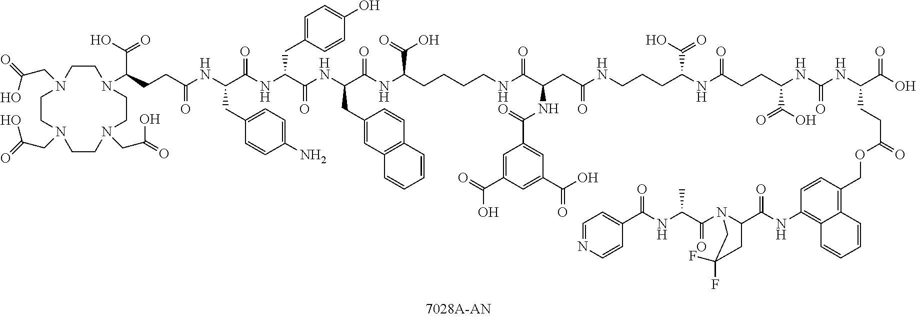 [SMILES: C[C@@H](NC(=O)c1ccncc1)C(=O)N1CC(F)(F)CC1C(=O)Nc1ccc(COC(=O)CC[C@H](NC(=O)N[C@@H](CCC(=O)N[C@H](CCCNC(=O)C[C@@H](NC(=O)c2cc(C(=O)O)cc(C(=O)O)c2)C(=O)NCCCC[C@@H](NC(=O)[C@@H](Cc2ccc3ccccc3c2)NC(=O)[C@@H](Cc2ccc(O)cc2)NC(=O)[C@H](Cc2ccc(N)cc2)NC(=O)CC[C@H](C(=O)O)N2CCN(CC(=O)O)CCN(CC(=O)O)CCN(CC(=O)O)CC2)C(=O)O)C(=O)O)C(=O)O)C(=O)O)c2ccccc12]

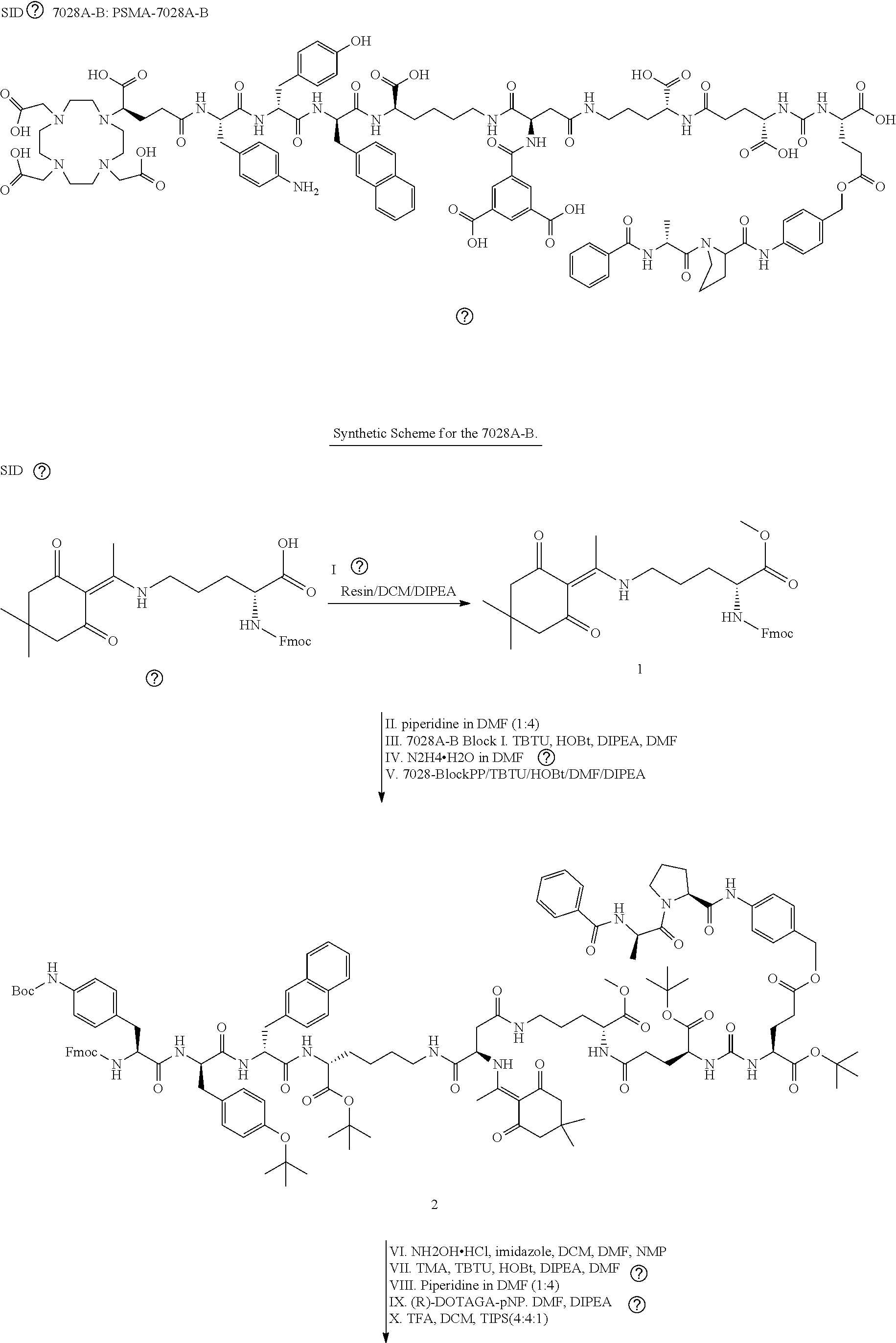

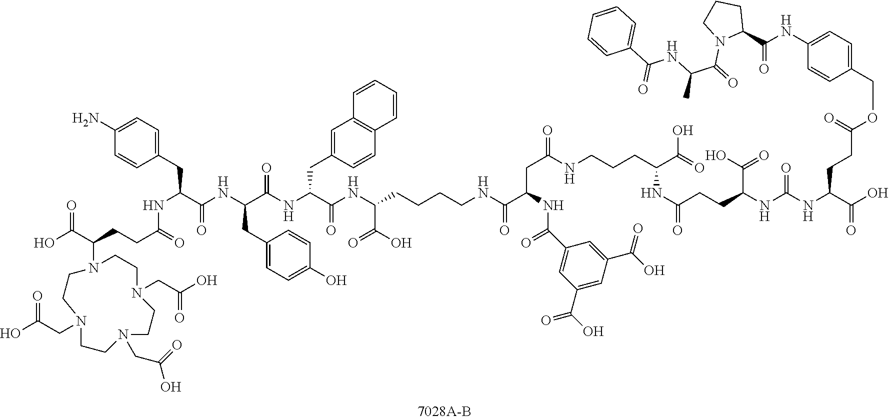 [SMILES: C[C@@H](NC(=O)c1ccccc1)C(=O)N1CCC[C@H]1C(=O)Nc1ccc(COC(=O)CC[C@H](NC(=O)N[C@@H](CCC(=O)N[C@H](CCCNC(=O)C[C@@H](NC(=O)c2cc(C(=O)O)cc(C(=O)O)c2)C(=O)NCCCC[C@@H](NC(=O)[C@@H](Cc2ccc3ccccc3c2)NC(=O)[C@@H](Cc2ccc(O)cc2)NC(=O)[C@H](Cc2ccc(N)cc2)NC(=O)CC[C@H](C(=O)O)N2CCN(CC(=O)O)CCN(CC(=O)O)CCN(CC(=O)O)CC2)C(=O)O)C(=O)O)C(=O)O)C(=O)O)cc1]

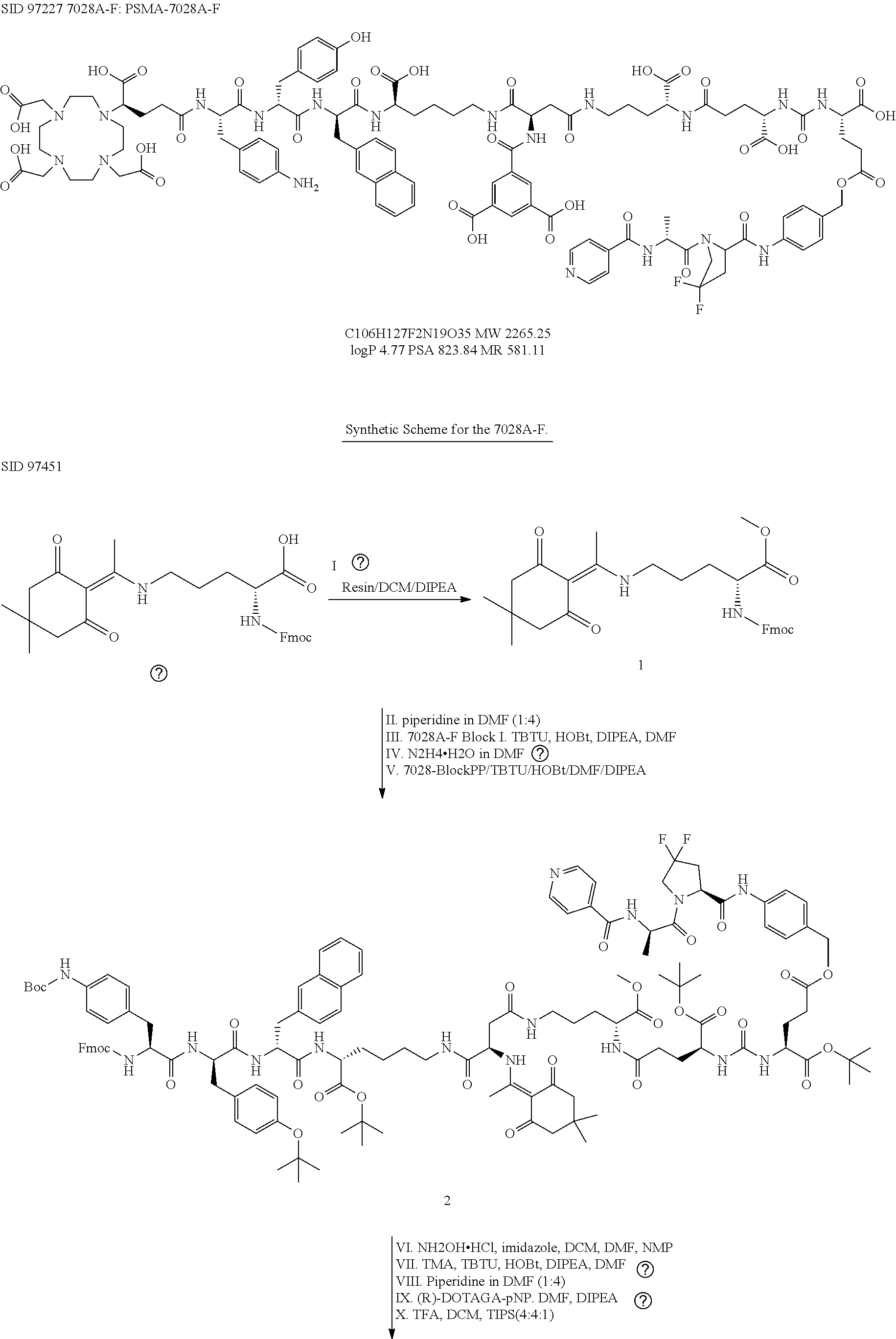

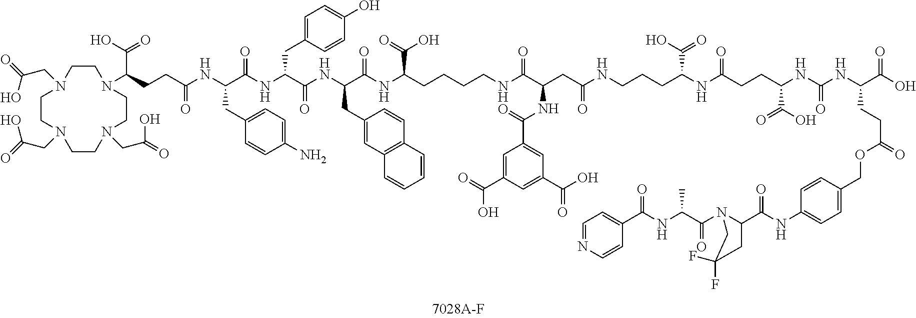 [SMILES: C[C@@H](NC(=O)c1ccncc1)C(=O)N1CC(F)(F)CC1C(=O)Nc1ccc(COC(=O)CC[C@H](NC(=O)N[C@@H](CCC(=O)N[C@H](CCCNC(=O)C[C@@H](NC(=O)c2cc(C(=O)O)cc(C(=O)O)c2)C(=O)NCCCC[C@@H](NC(=O)[C@@H](Cc2ccc3ccccc3c2)NC(=O)[C@@H](Cc2ccc(O)cc2)NC(=O)[C@H](Cc2ccc(N)cc2)NC(=O)CC[C@H](C(=O)O)N2CCN(CC(=O)O)CCN(CC(=O)O)CCN(CC(=O)O)CC2)C(=O)O)C(=O)O)C(=O)O)C(=O)O)cc1]

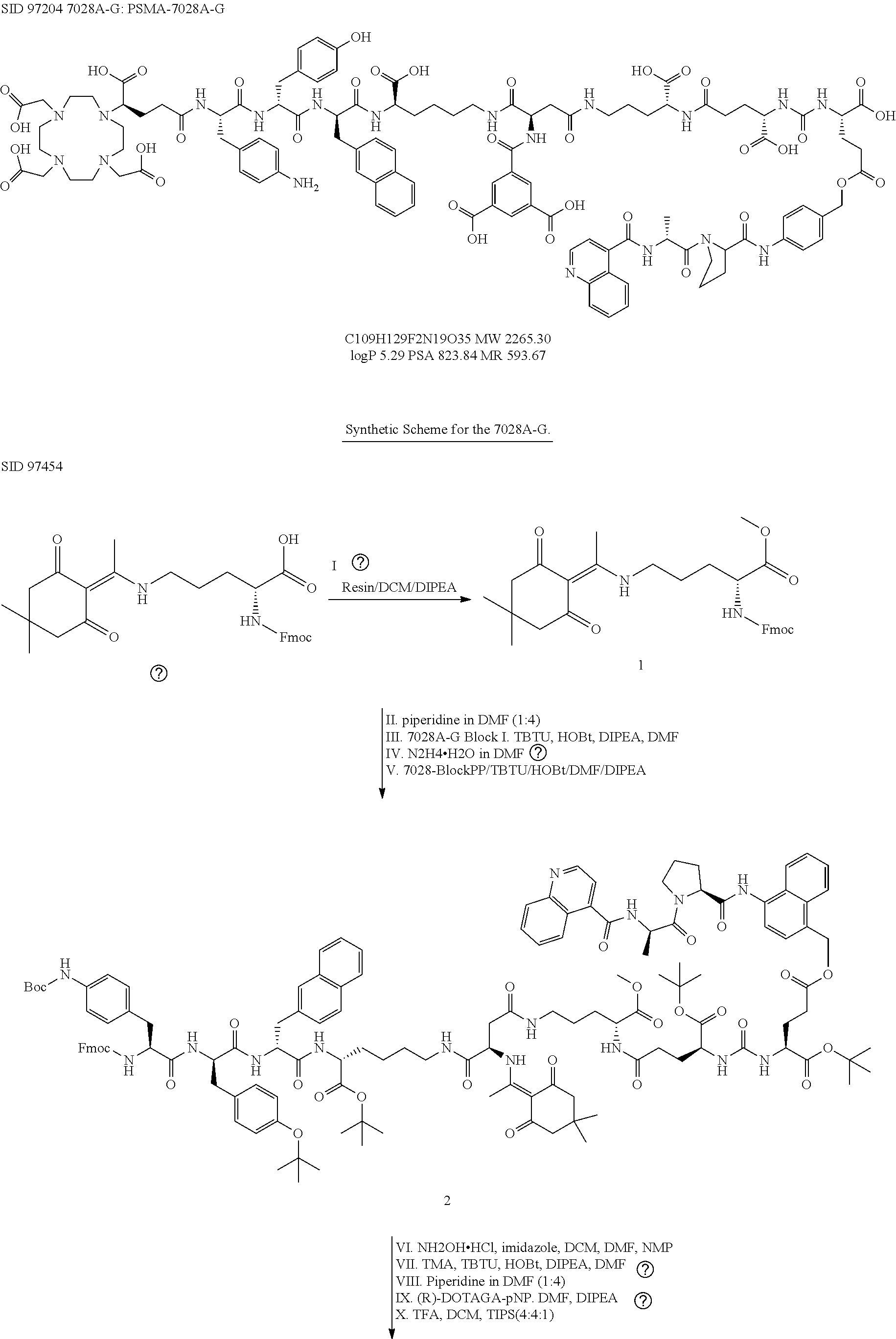

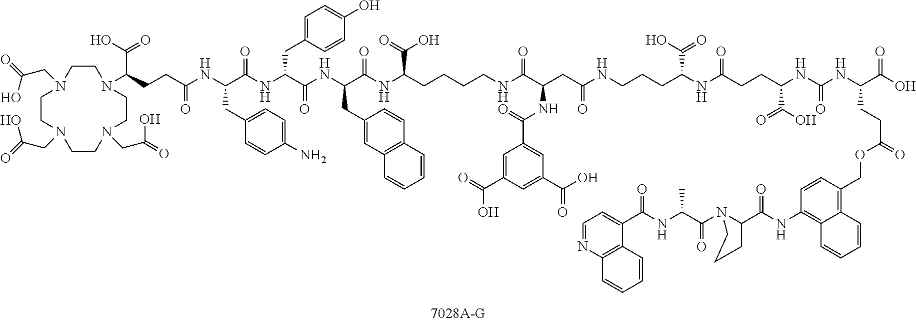 [SMILES: C[C@@H](NC(=O)c1ccnc2ccccc12)C(=O)N1CCCC1C(=O)Nc1ccc(COC(=O)CC[C@H](NC(=O)N[C@@H](CCC(=O)N[C@H](CCCNC(=O)C[C@@H](NC(=O)c2cc(C(=O)O)cc(C(=O)O)c2)C(=O)NCCCC[C@@H](NC(=O)[C@@H](Cc2ccc3ccccc3c2)NC(=O)[C@@H](Cc2ccc(O)cc2)NC(=O)[C@H](Cc2ccc(N)cc2)NC(=O)CC[C@H](C(=O)O)N2CCN(CC(=O)O)CCN(CC(=O)O)CCN(CC(=O)O)CC2)C(=O)O)C(=O)O)C(=O)O)C(=O)O)c2ccccc12]

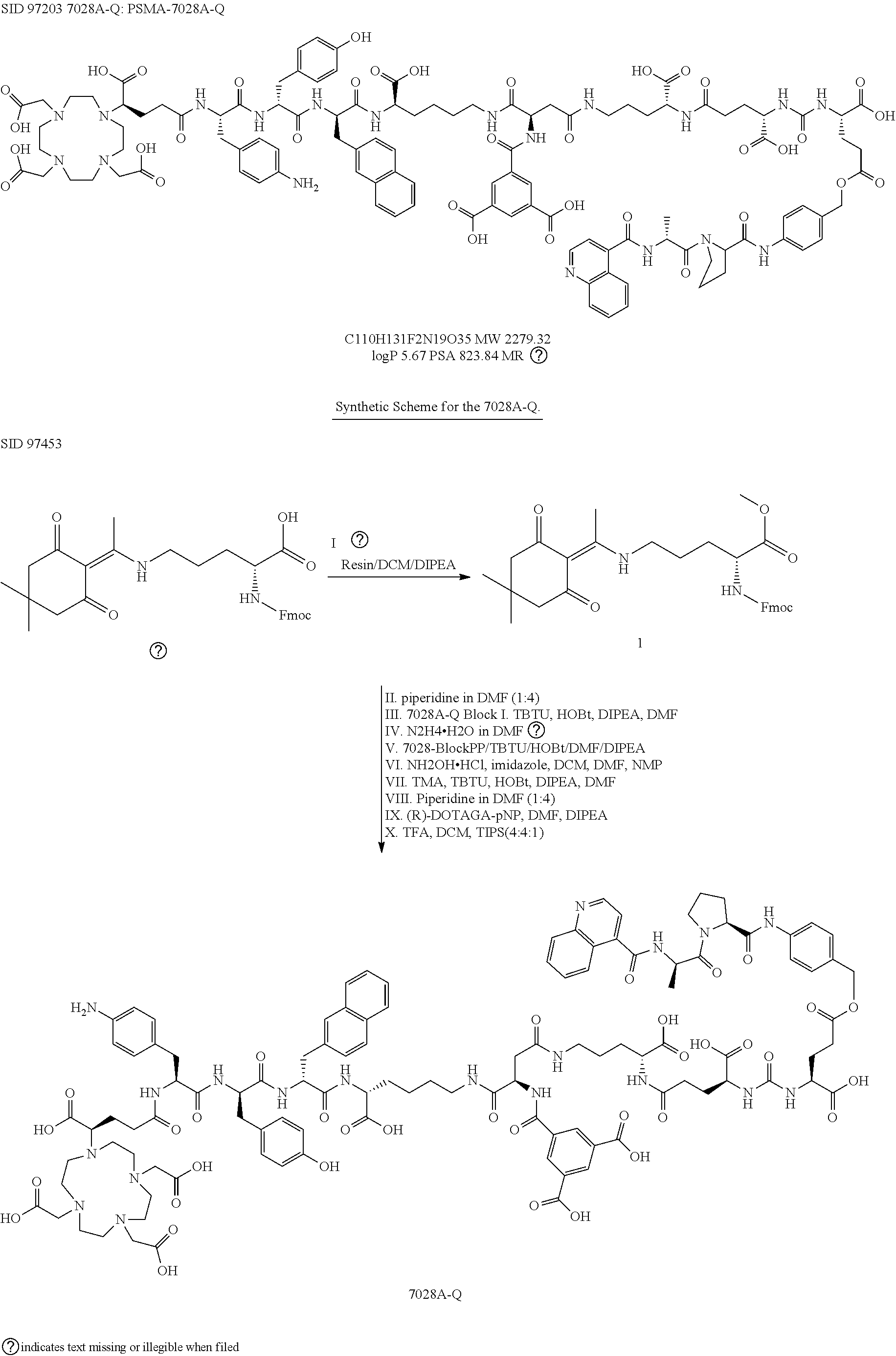

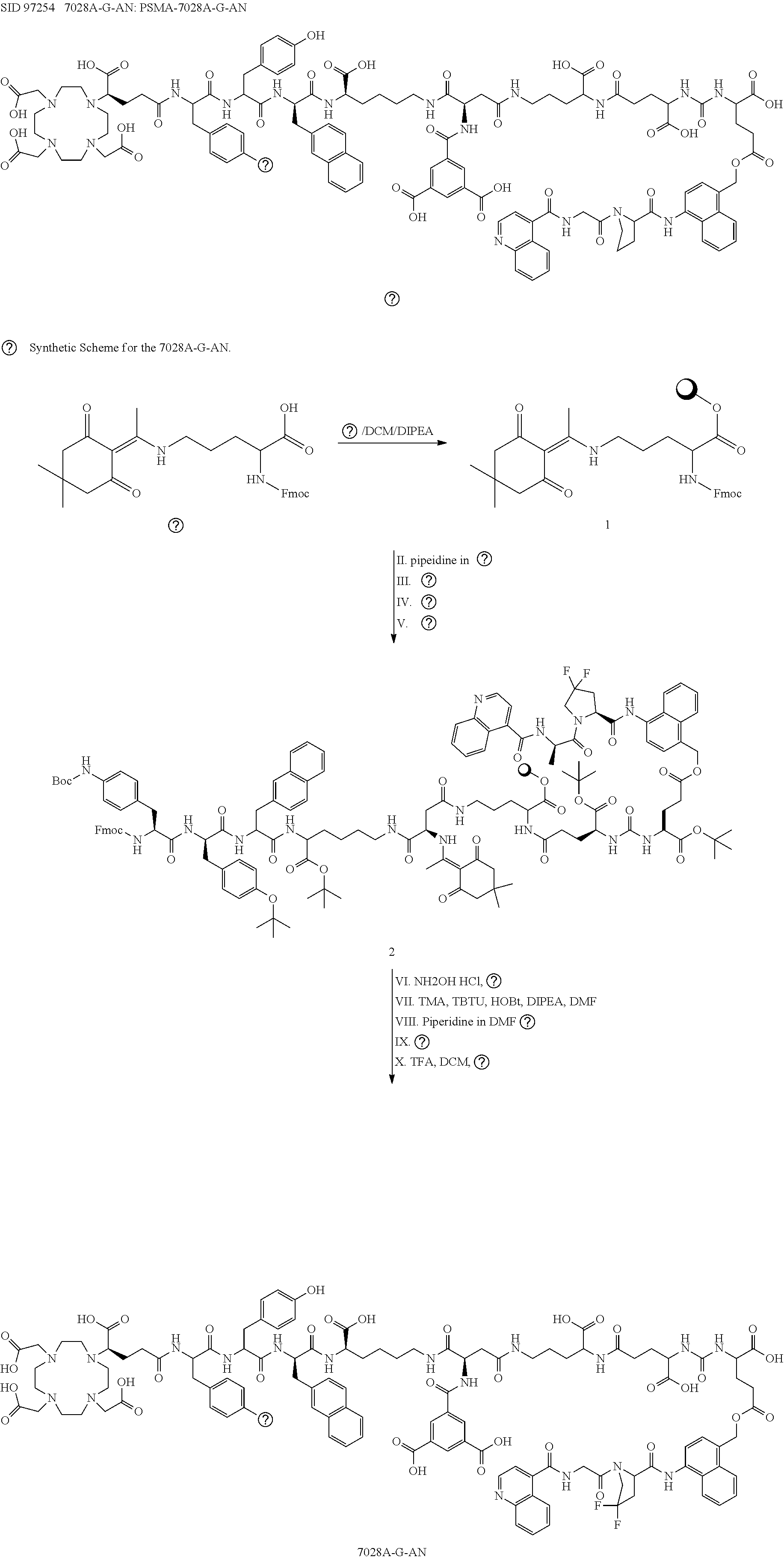

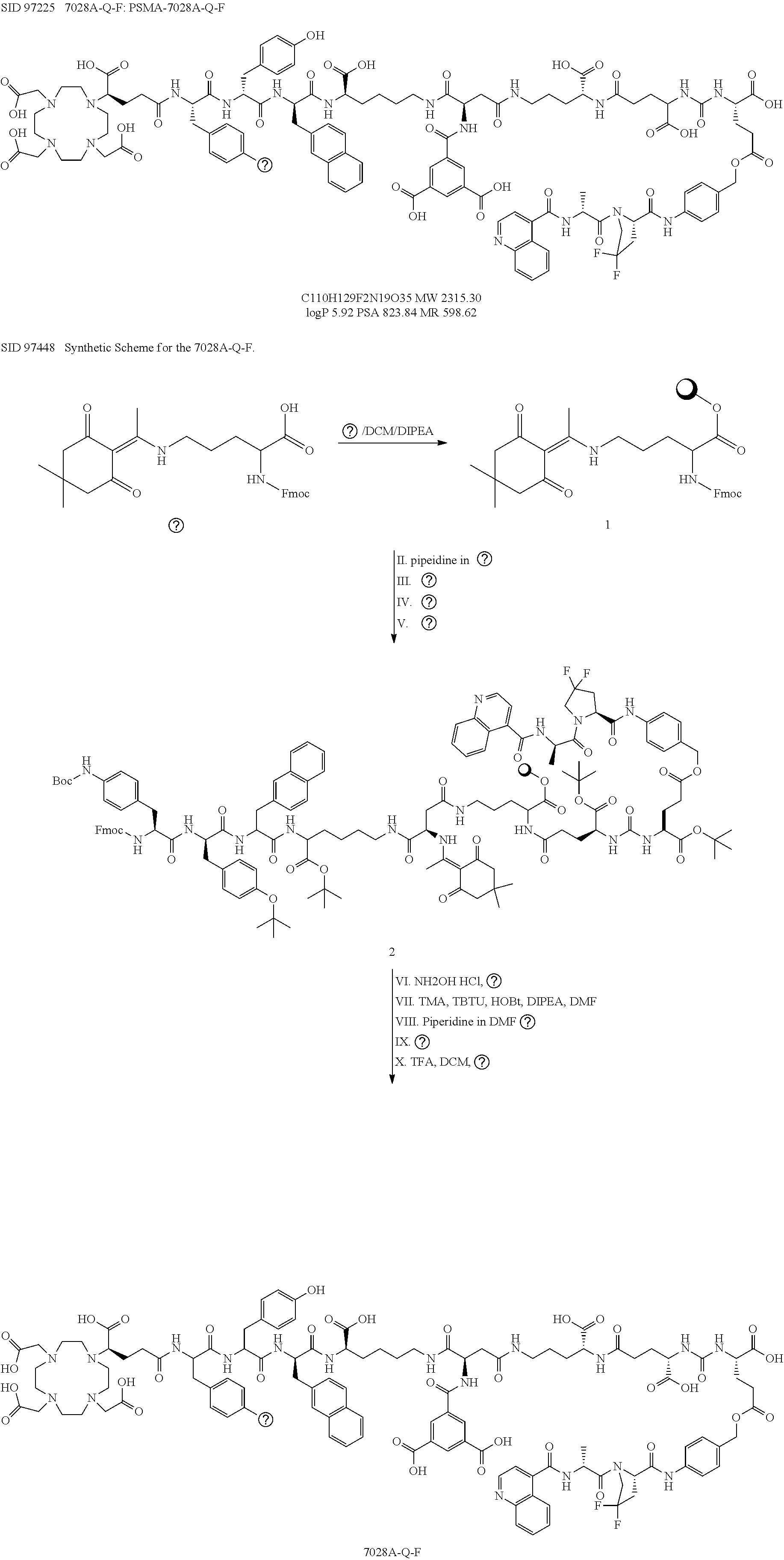

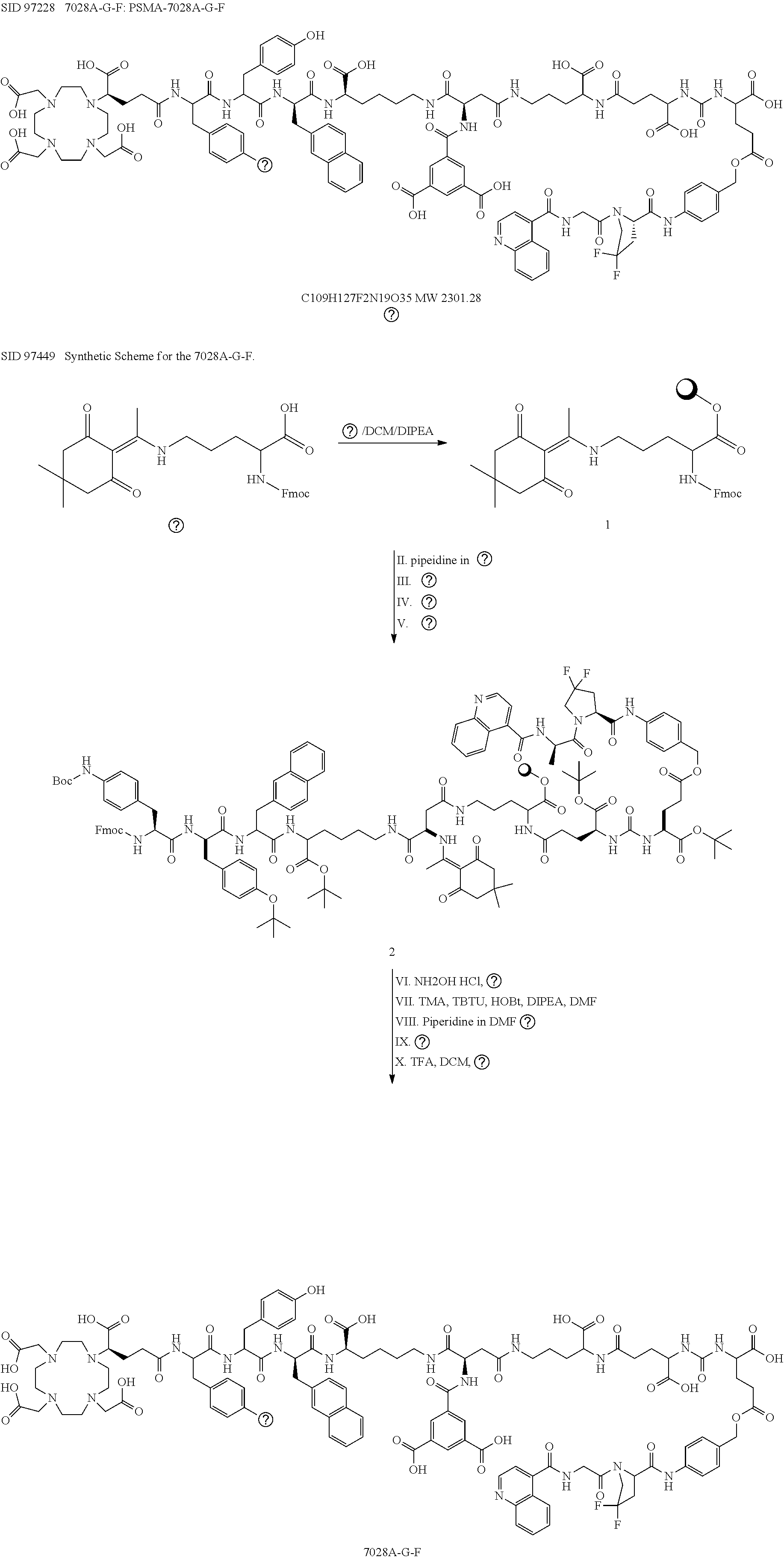

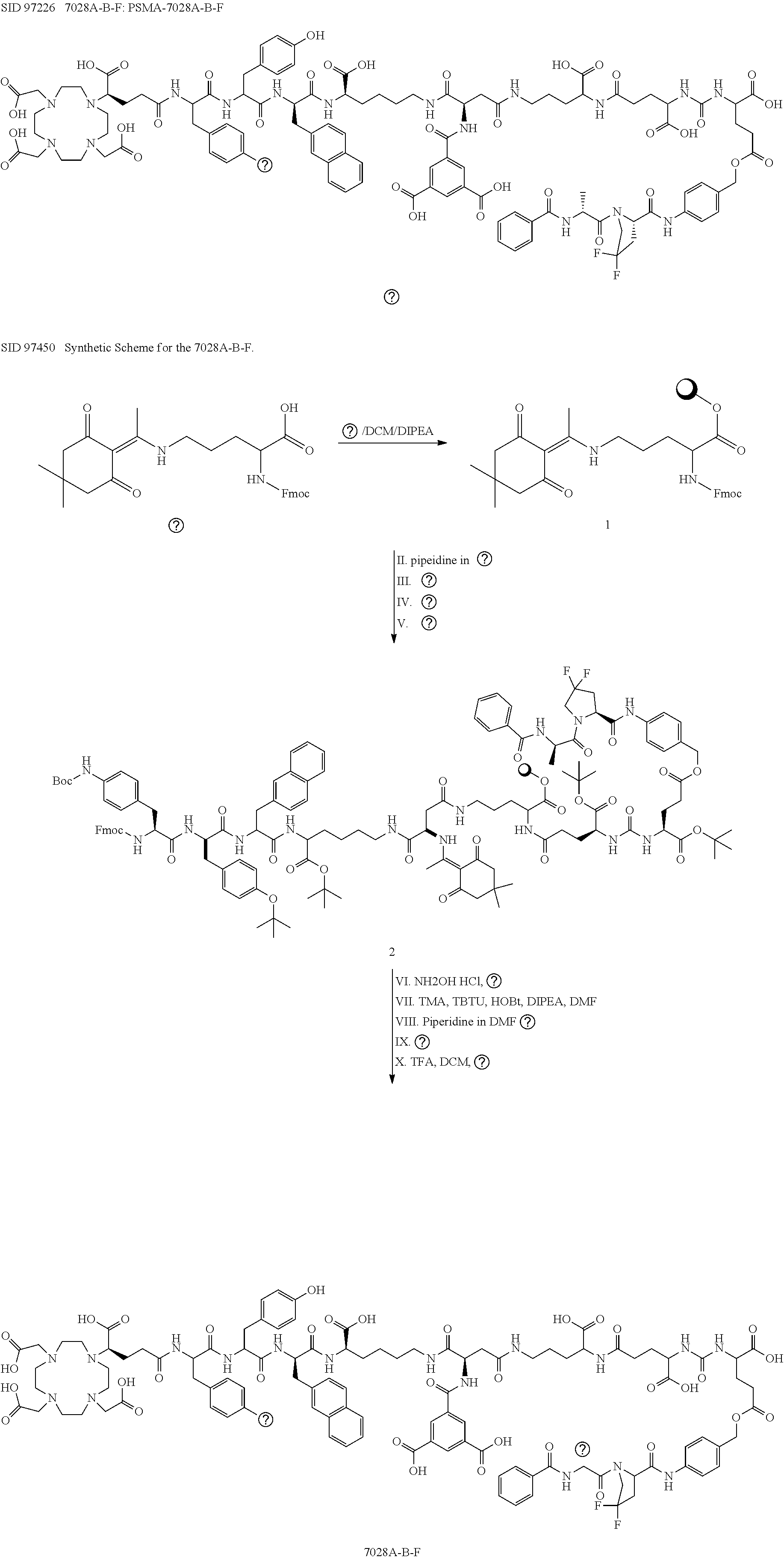

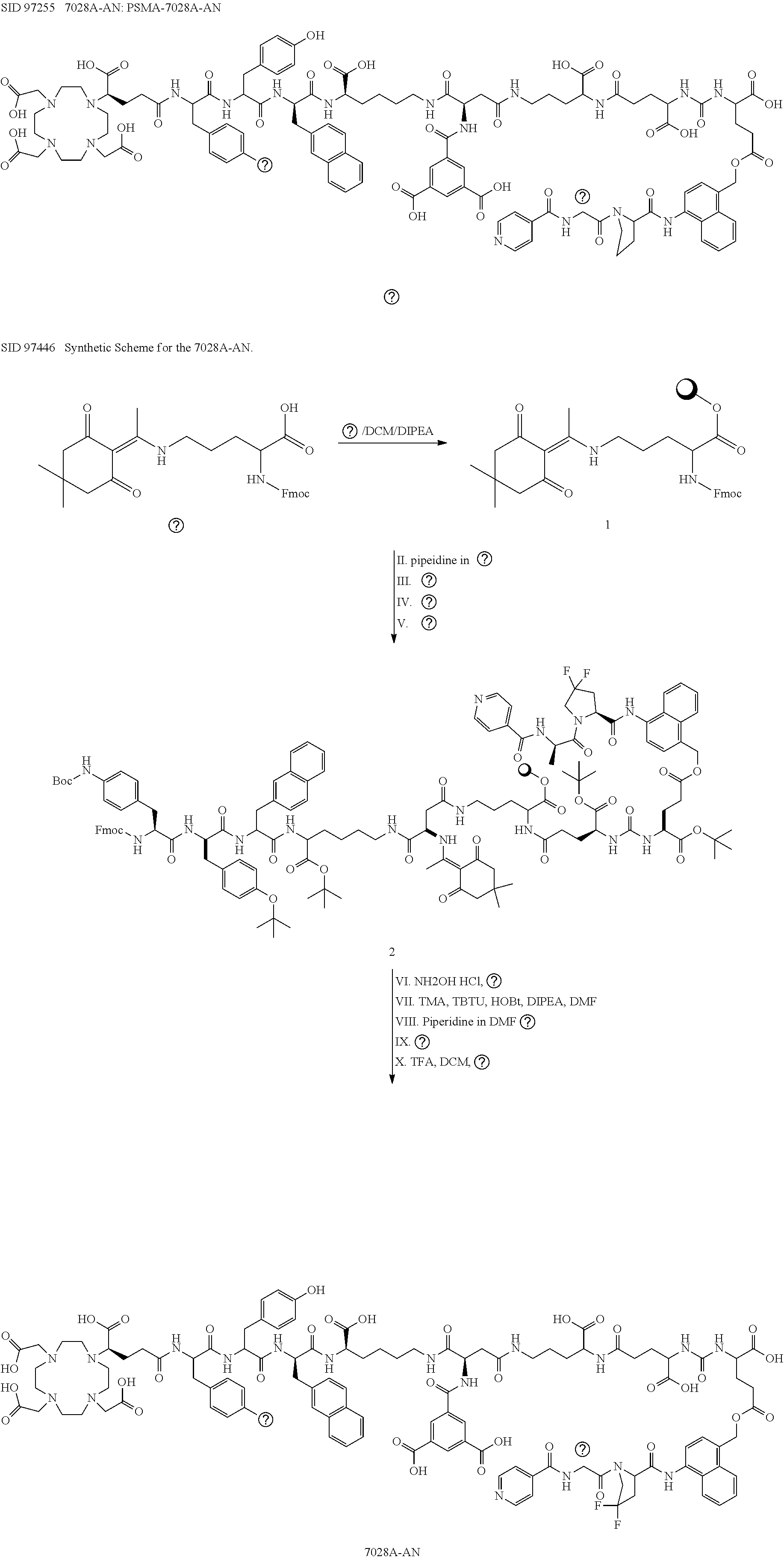

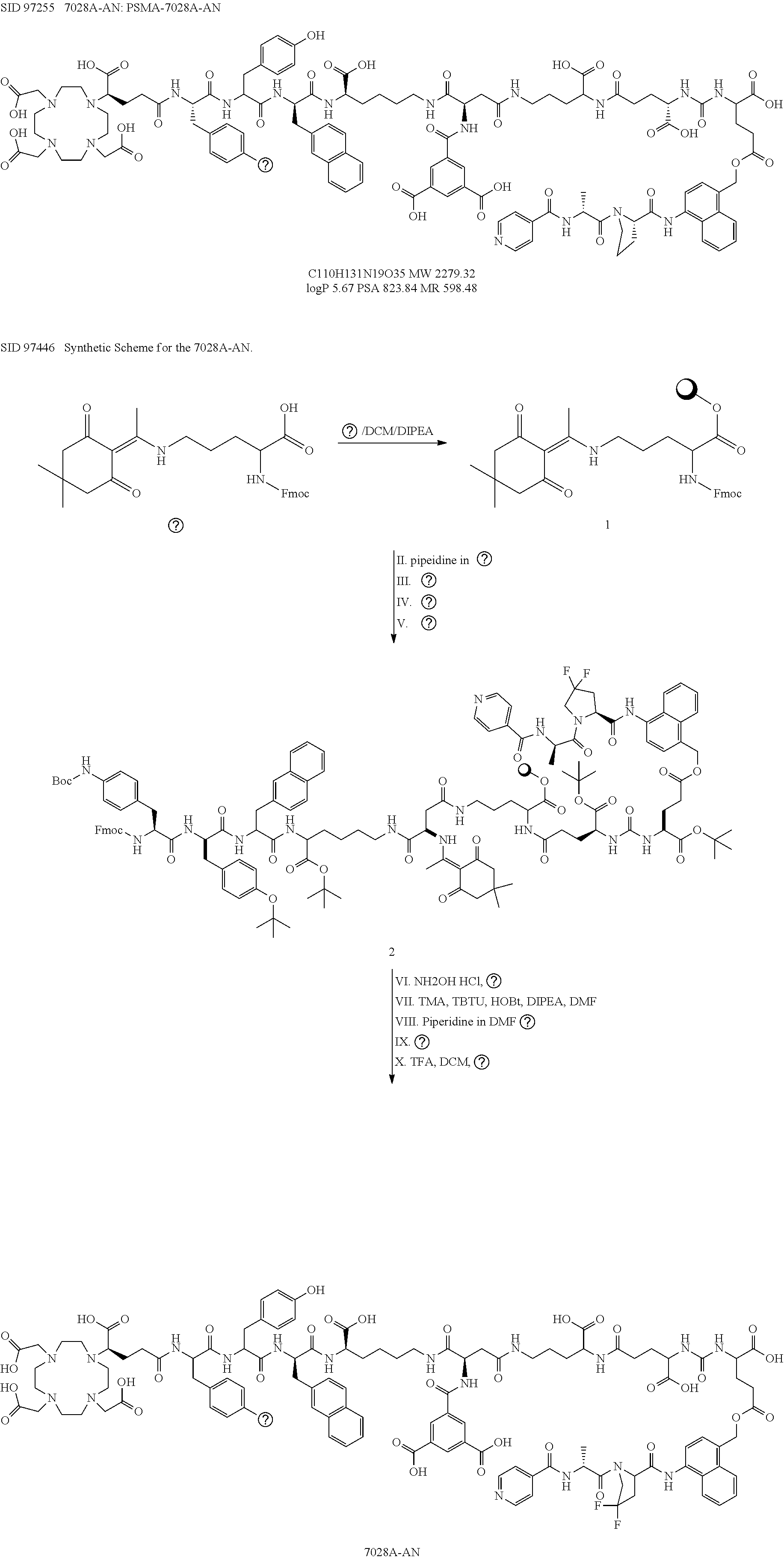

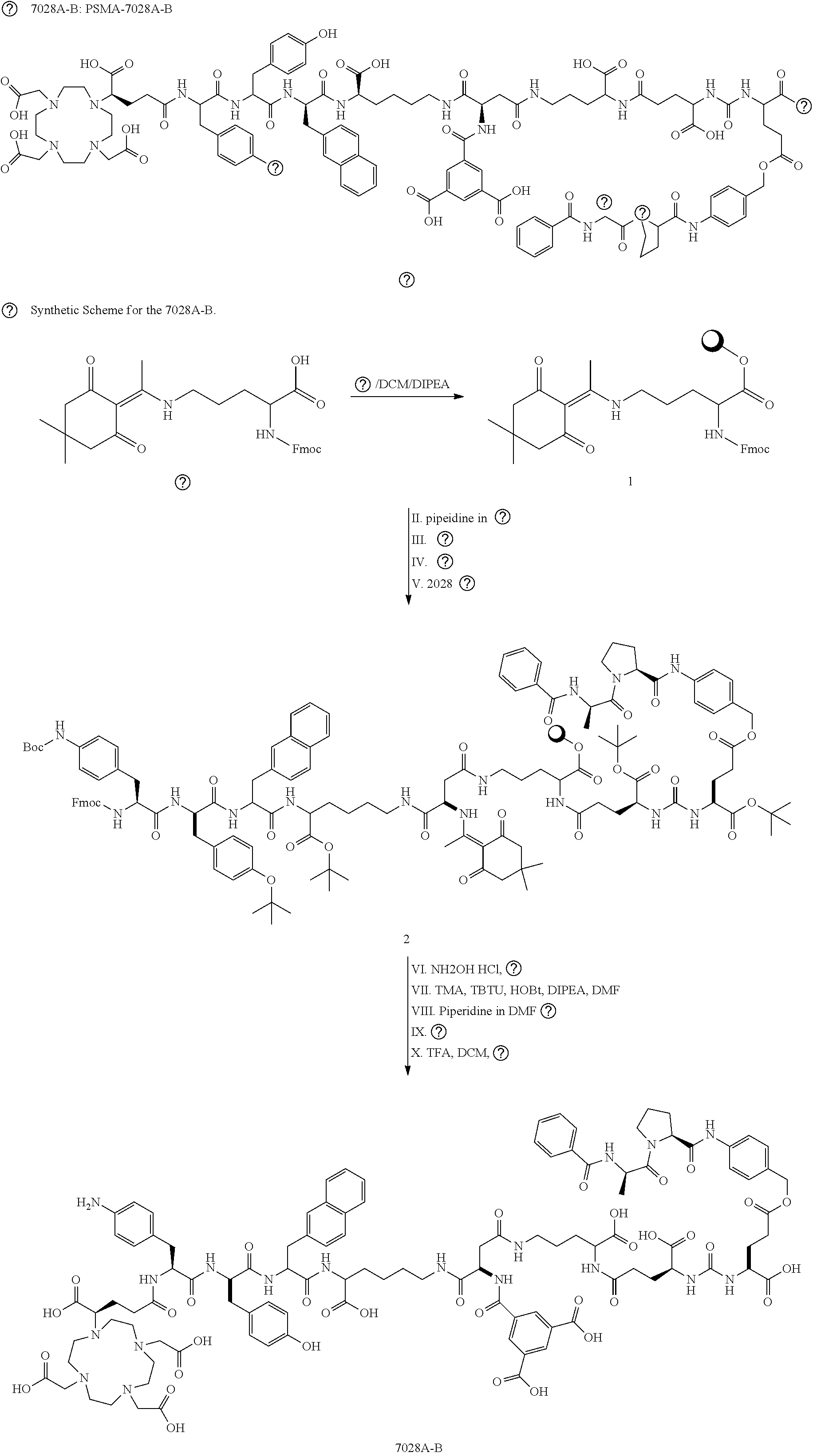

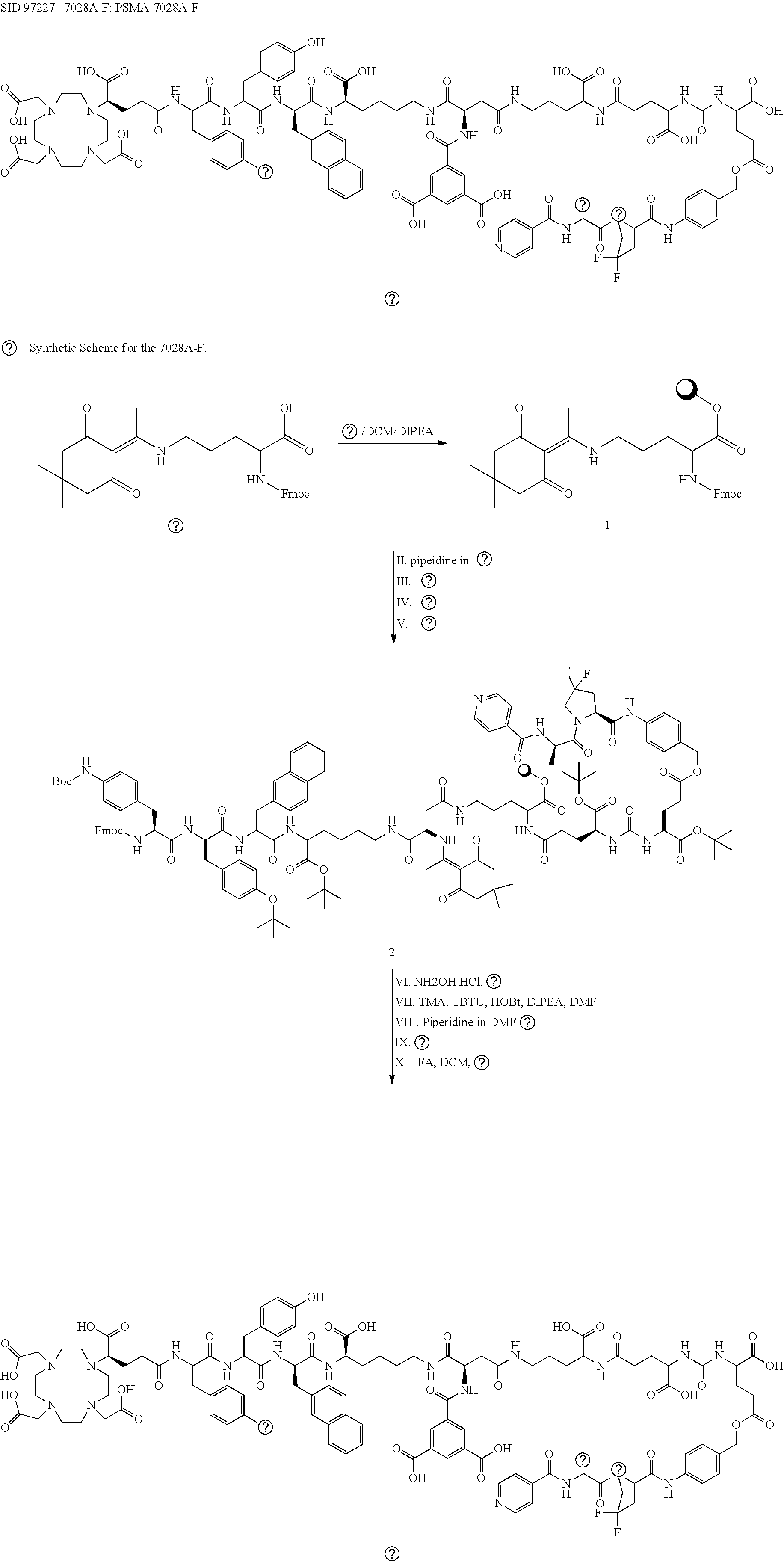

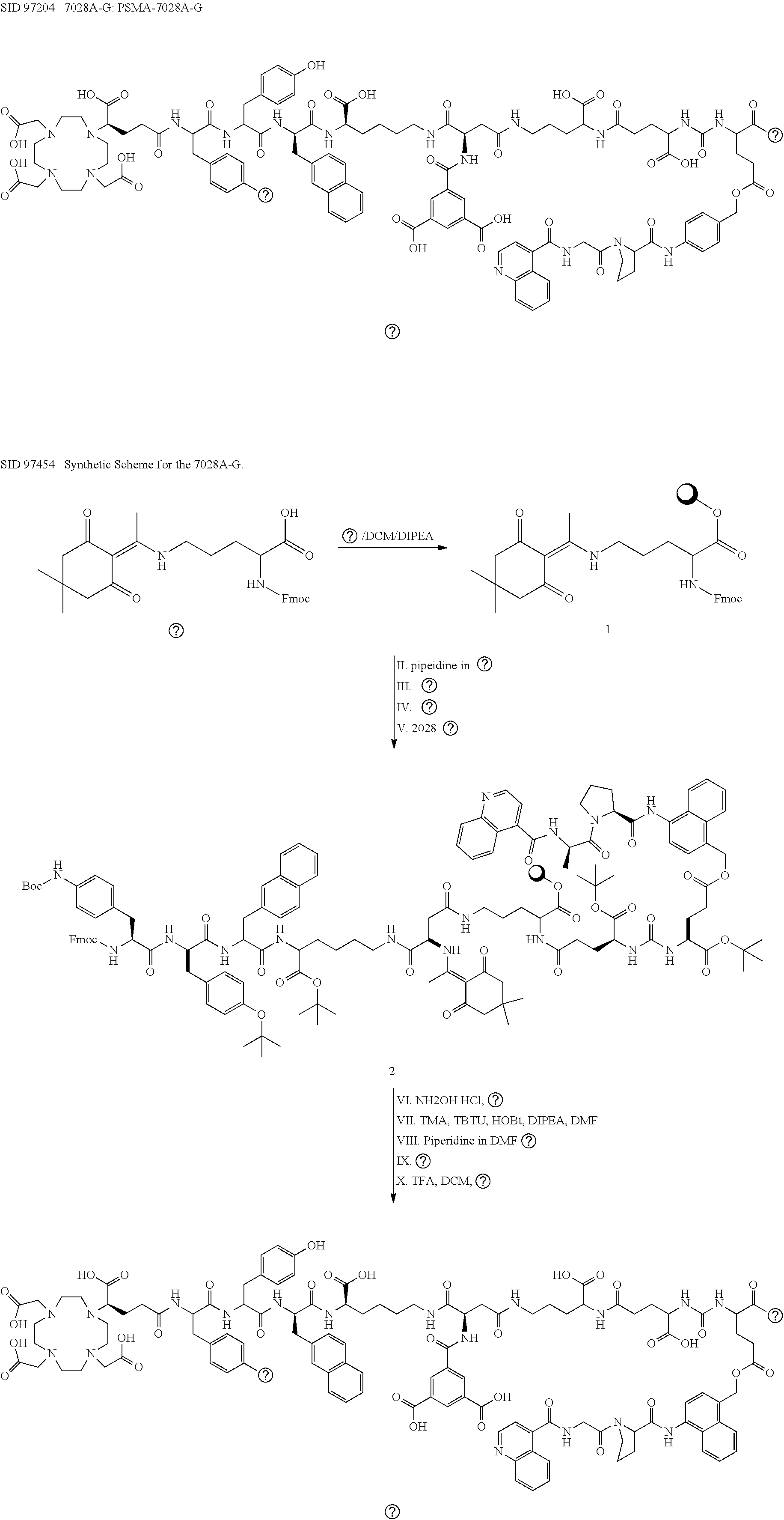

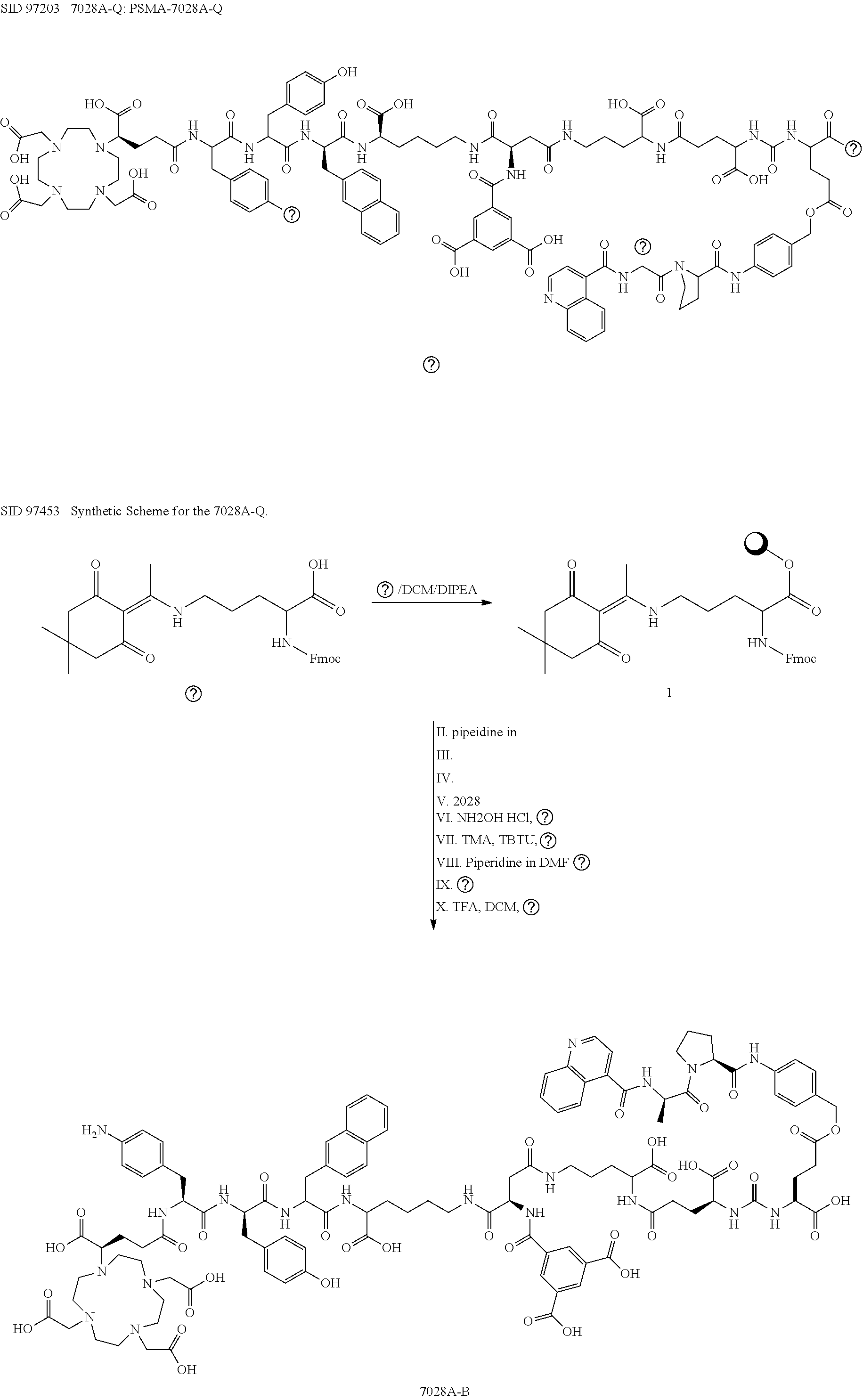

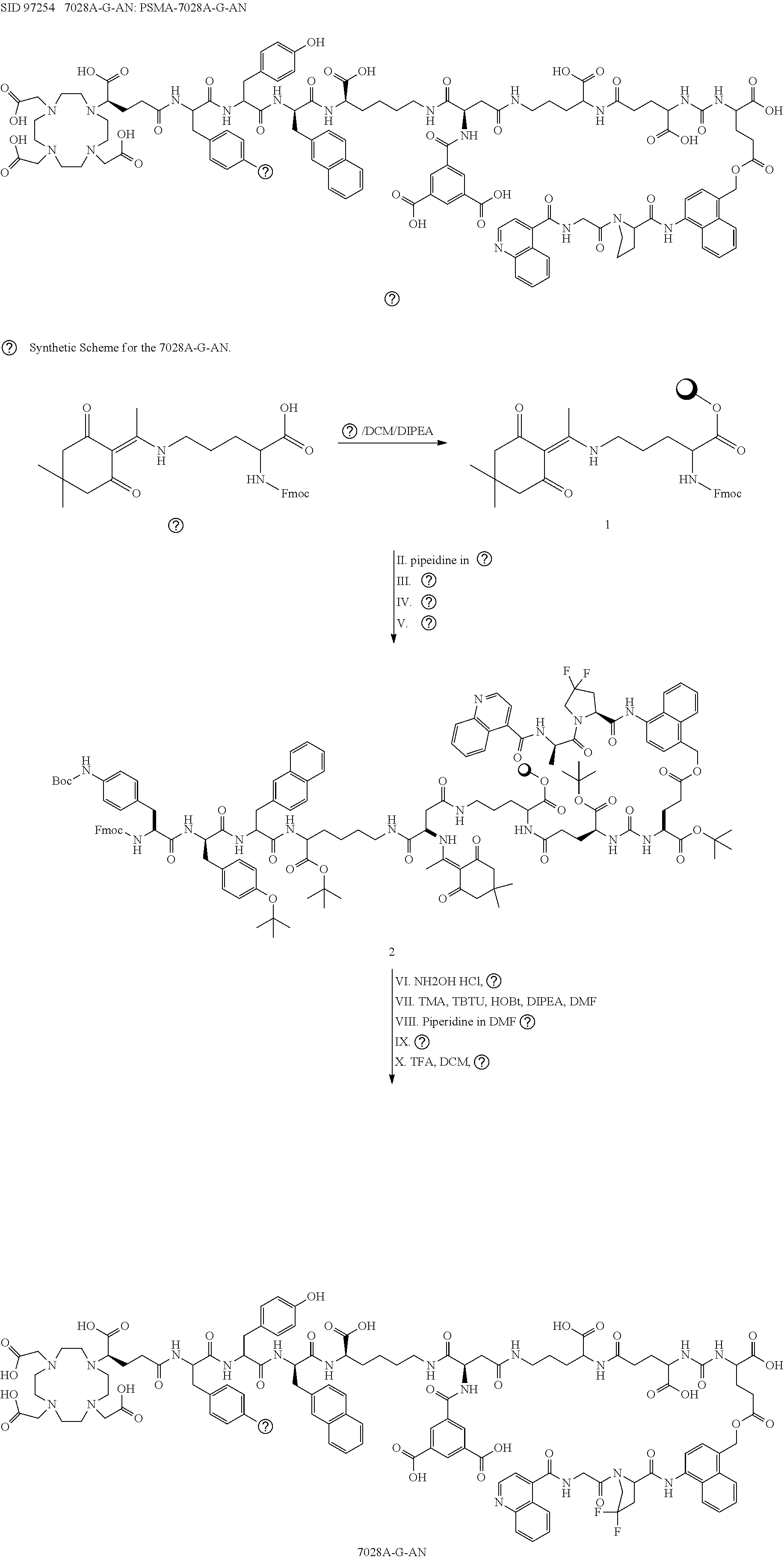

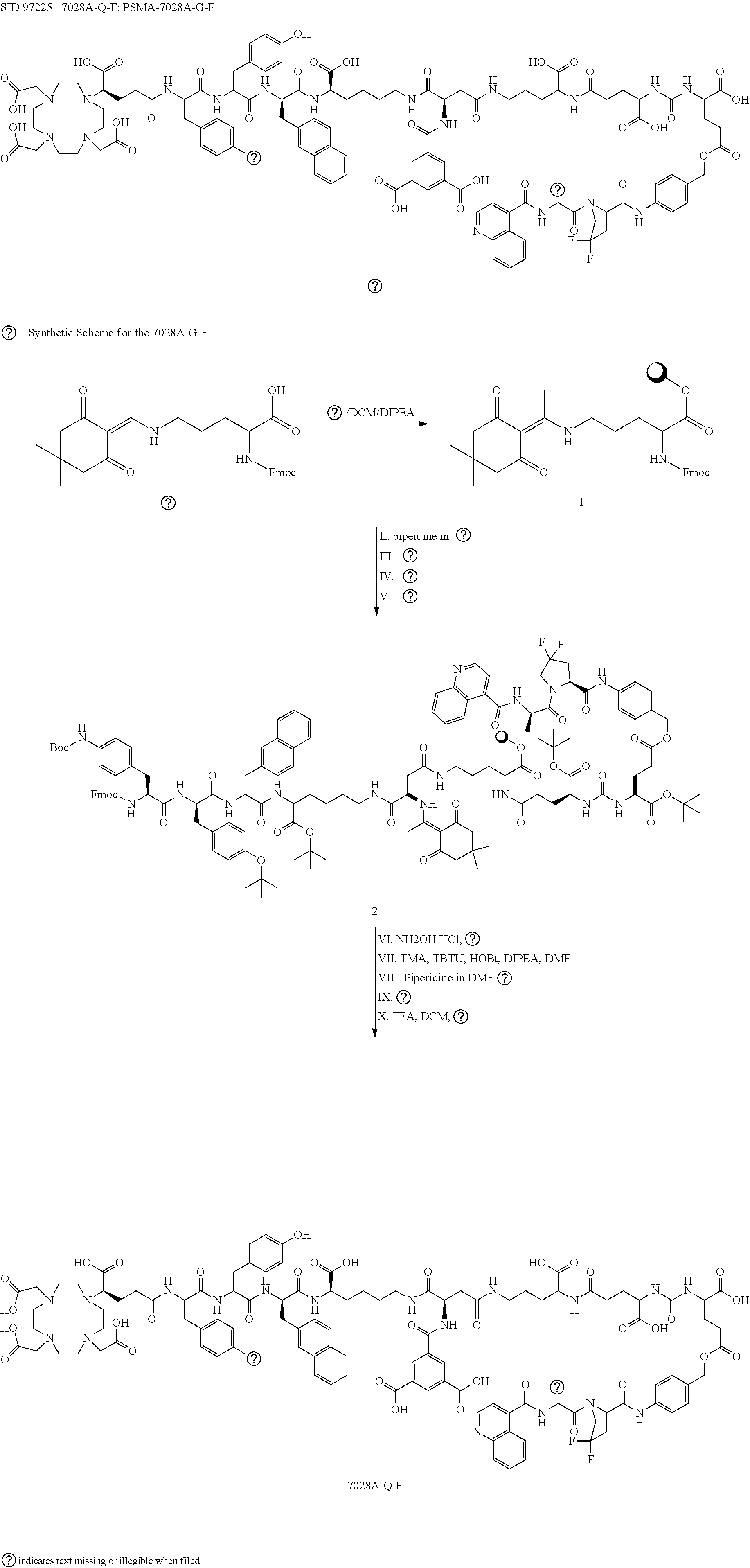

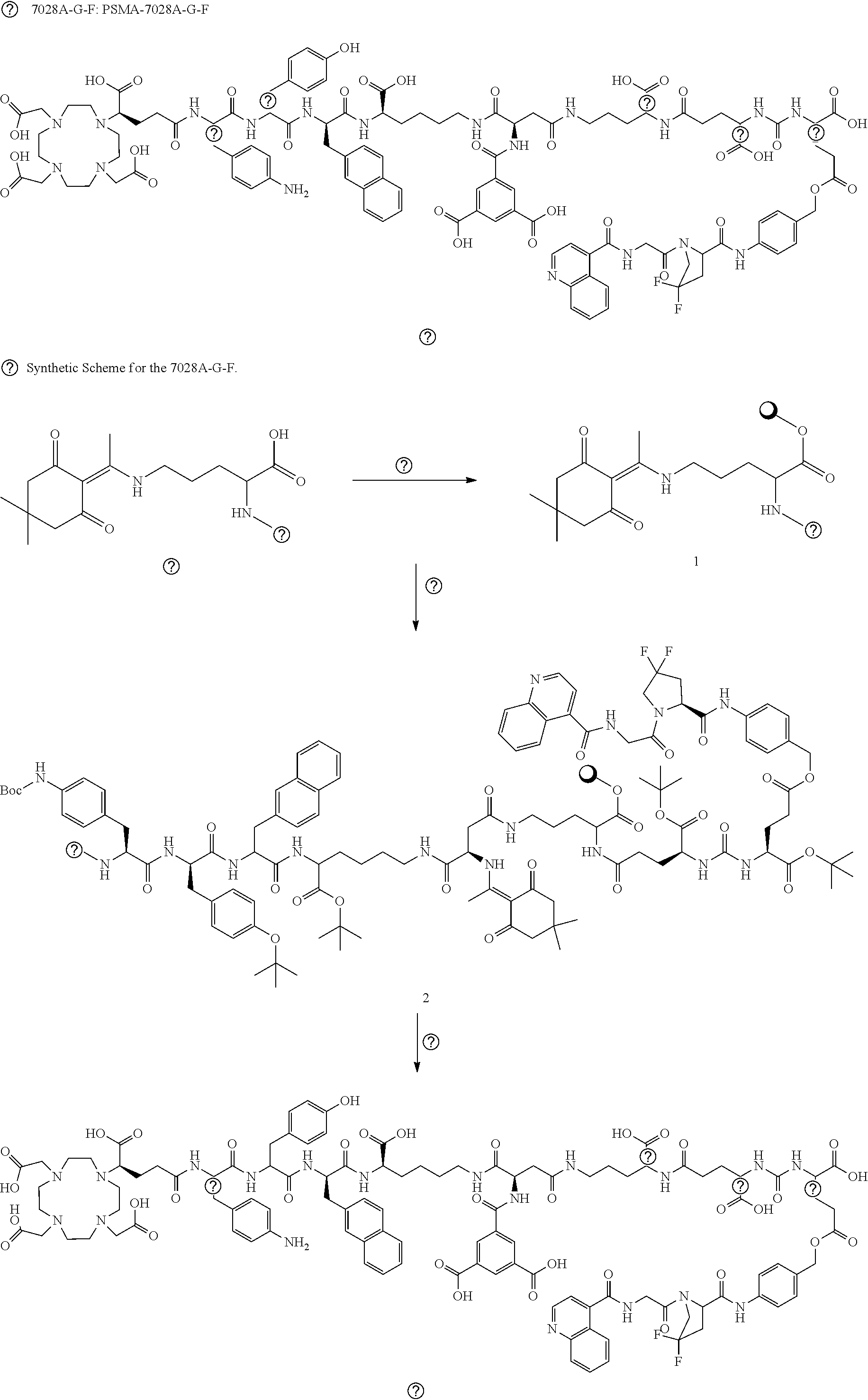

### Example 7: IC50 Assay of 7028P and FAP-Activated 7028A, 7028B and 7028C

Objective: The objective of this study was to demonstrate affinity of 7028P and 7028A/B/C for binding to prostate specific antigen (PSMA) by measuring the inhibition of PSMA enzyme activity. Enzyme activity was measured with Acetyl-Asp-Glu as the substrate and monitoring the production of the primary amino group generated by the enzymatic cleavage of the peptide bond. The amino group is detected using fluoroaldehyde o-phthaldedialdehyde which along with mercaptoethanol forms a fluorescent adduct with primary amines.

The structures of 7028P & 7028 A/B/C are shown below

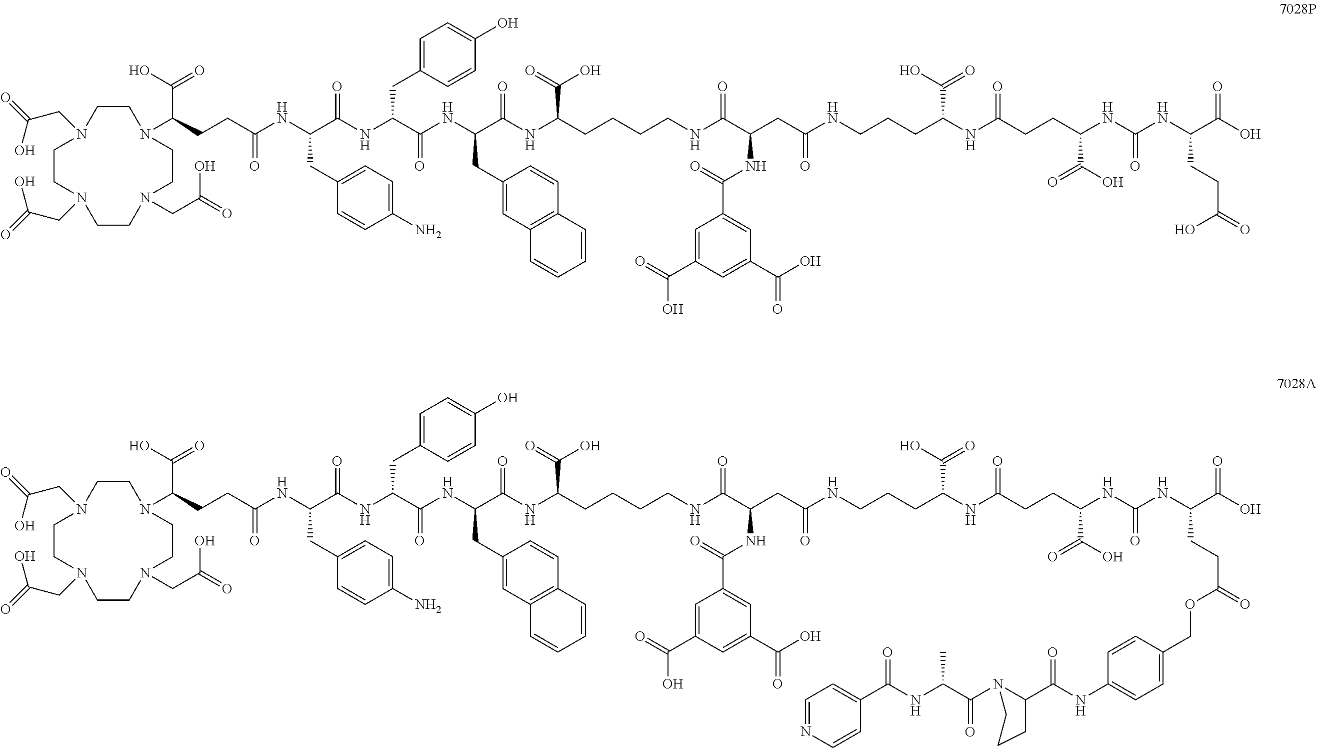

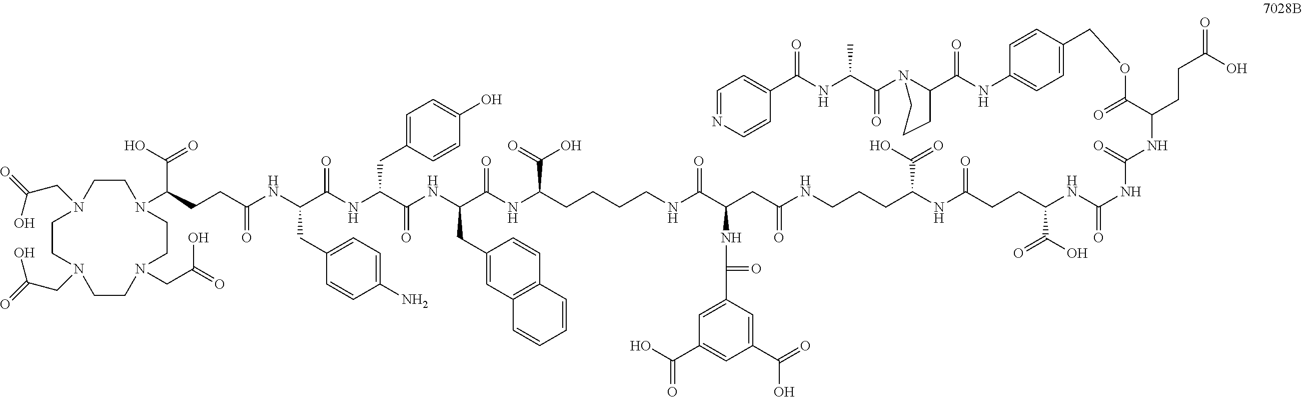 [SMILES: C[C@@H](NC(=O)c1ccncc1)C(=O)N1CCCC1C(=O)Nc1ccc(COC(=O)C(CCC(=O)O)NC(=O)NC(=O)N[C@@H](CCC(=O)N[C@H](CCCNC(=O)C[C@@H](NC(=O)c2cc(C(=O)O)cc(C(=O)O)c2)C(=O)NCCCC[C@@H](NC(=O)[C@@H](Cc2ccc3ccccc3c2)NC(=O)[C@@H](Cc2ccc(O)cc2)NC(=O)[C@H](Cc2ccc(N)cc2)NC(=O)CC[C@H](C(=O)O)N2CCN(CC(=O)O)CCN(CC(=O)O)CCN(CC(=O)O)CC2)C(=O)O)C(=O)O)C(=O)O)cc1]

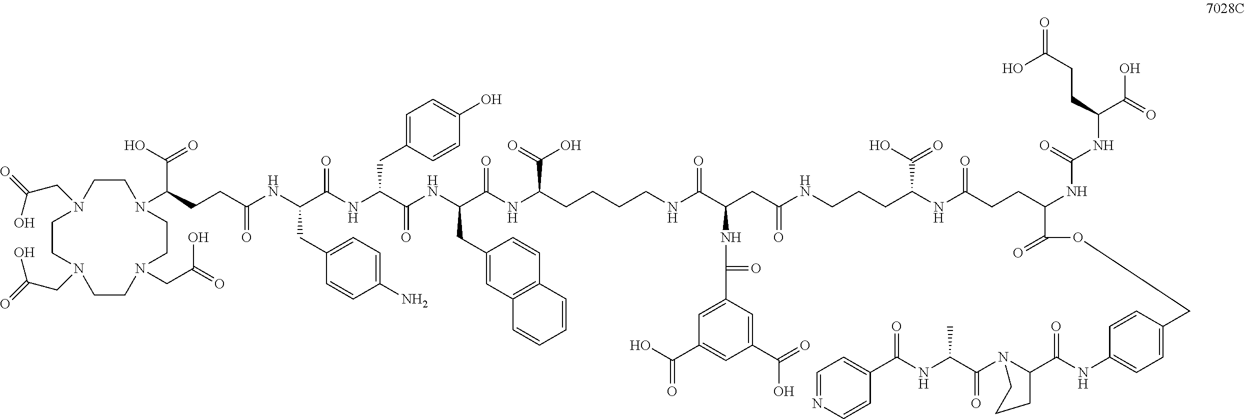 [SMILES: C[C@@H](NC(=O)c1ccncc1)C(=O)N1CCCC1C(=O)Nc1ccc(COC(=O)C(CCC(=O)N[C@H](CCCNC(=O)C[C@@H](NC(=O)c2cc(C(=O)O)cc(C(=O)O)c2)C(=O)NCCCC[C@@H](NC(=O)[C@@H](Cc2ccc3ccccc3c2)NC(=O)[C@@H](Cc2ccc(O)cc2)NC(=O)[C@H](Cc2ccc(N)cc2)NC(=O)CC[C@H](C(=O)O)N2CCN(CC(=O)O)CCN(CC(=O)O)CCN(CC(=O)O)CC2)C(=O)O)C(=O)O)NC(=O)N[C@@H](CCC(=O)O)C(=O)O)cc1]

### Methodology

### Materials:

rhPSMA (R&D systems, 4334-ZN-010
Assay buffer: 50 mM HEPES pH 7.5, 100 mM NaCl
Ac-Asp-Glu (Sigma, A5930)
2-PMPA (2-(Phosphonomethyl)-pentandioic acid, Sigma, SML1612)
Fluoroaldehyde o-phthaldedialdehyde (Fluoroaldehyde OPA, Thermo Fisher Scientific, 26025
7028P, 7028A, 7028B & 7028C (Tufts)
Corning 96 well flat bottom polystruyrene NBS (Fisher, 07-201-203

### Equipment:

Molecular Devices M2e plate reader
Procedures:
1. rhPSMA was dissolved in assay buffer at 0.4 μg/mL.
2. Ac-Asp-Glu was dissolved in assay buffer at 80 μM
3. Stock solutions of inhibitors (7028P, 7028A, 7028B & 7028C) were prepared at 100 μM in DMSO.
4. Inhibitor stock was diluted by addition of 160 μL to 240 μL of assay buffer for a 40 μM working stock. A series of 10× dilutions were fone to prepare 4× concentrated samples of each inhibitor as shown below.

| | |
|---|---|
| Inhibitor Conc. | Inhibitor Reaction |
| (4x rxn conc.) | Conc. |

| | | | |
|---|---|---|---|
| 4.00E−06 | M | 1.00E−06 | M |
| 4.00E−07 | M | 1.00E−07 | M |
| 4.00E−08 | M | 1.00E−08 | M |
| 4.00E−09 | M | 1.00E−09 | M |
| 4.00E−10 | M | 1.00E−10 | M |
| 4.00E−11 | M | 1.00E−11 | M |
| 4.00E−12 | M | 1.00E−12 | M |
| 4.00E−13 | M | 1.00E−13 | M |
| 4.00E−14 | M | 1.00E−14 | M |
| 4.00E−15 | M | 1.00E−15 | M |
| 4.00E−16 | M | 1.00E−16 | M |

5. Inhibitor and substrate were mixed by combining 100 μL inhibitor with 100 μL of 80 μM substrate.
6. The reaction was started by addition of an equal volume (200 μL) of 0.4 μg/mL rhPMSA. Reaction mictures were incubated at 37° C. for 60 minutes.
   a. Enzyme concentration in reaction: 0.2 μg/mL
   b. Substrate concentration in reaction: 20 μM.
7. A blank sample containing only assay buffer and substrate, and a no-inhibitor control reaction sample with no inhibitor (100 μL buffer with 100 μL of 80 μM substrate and 200μΛ of 0.4 mg/μL rhPSMA) were prepared and reated as the inhibitor samples.
8. The reaction was stopped by heating in a boiling water bath for 5 minutes.
9. 100 μL of each sample was put in the wells of a 96 well plate in triplicate.
10. 100 μL of Fluoroaldehyde OPA reagent was added to each well and mixed.
11. Fluorescence was measured with excitation 330 nm and emission 450 nm.
12. The data was normalized to the largest value and the IC50 was determined using the “log(inhibitor) vs. response (3 parameter)” equation in Prism 9.

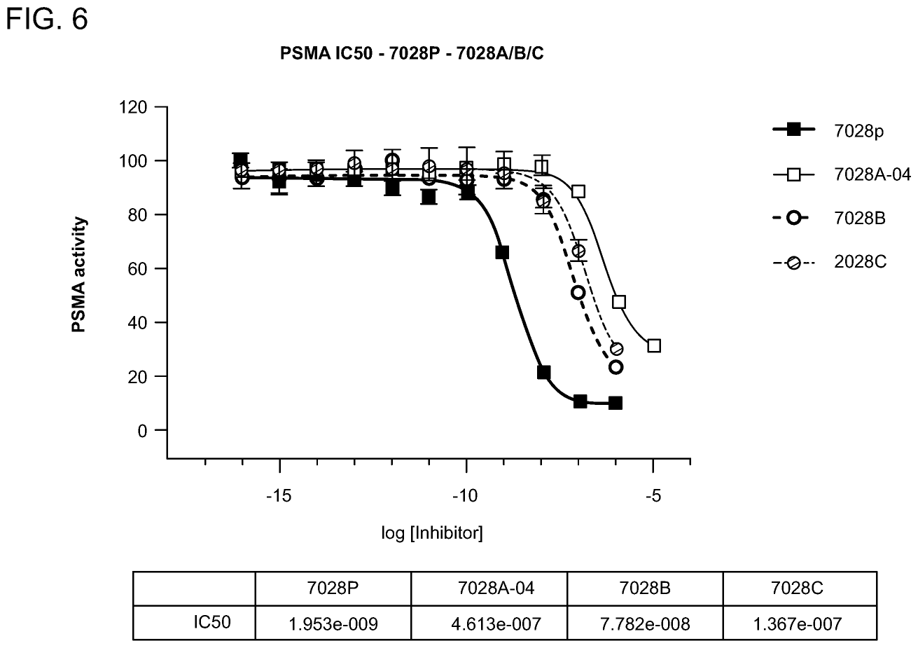

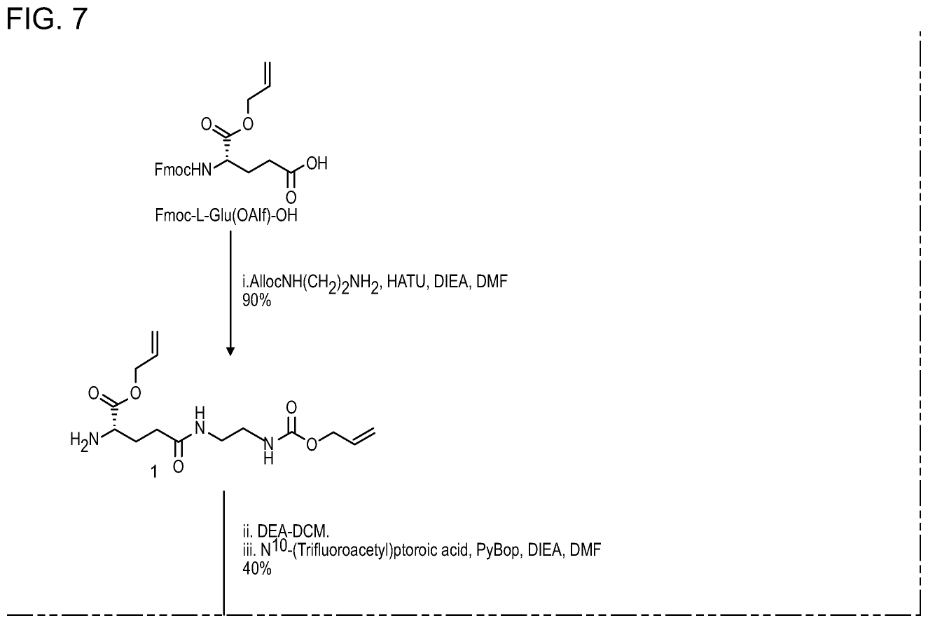

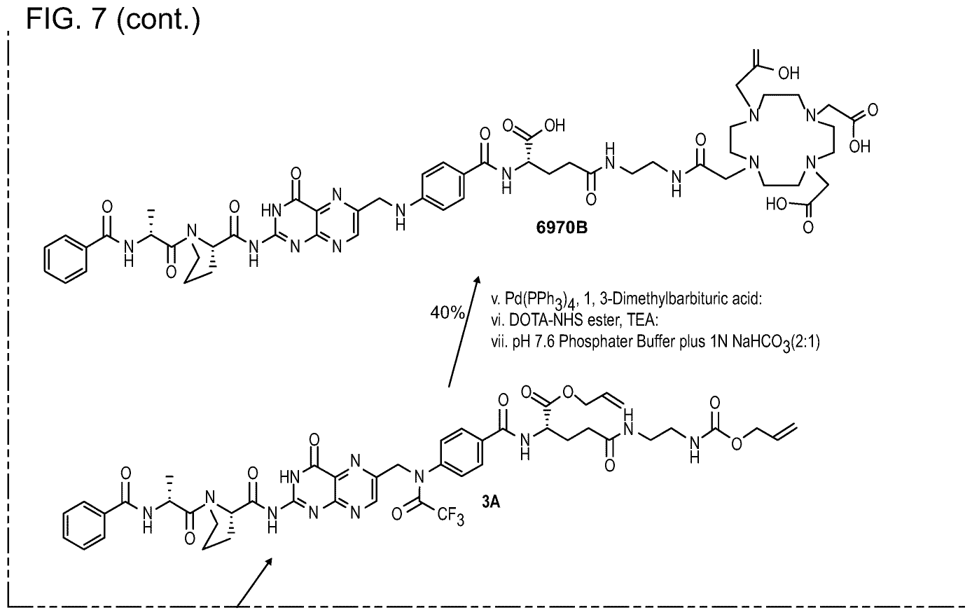

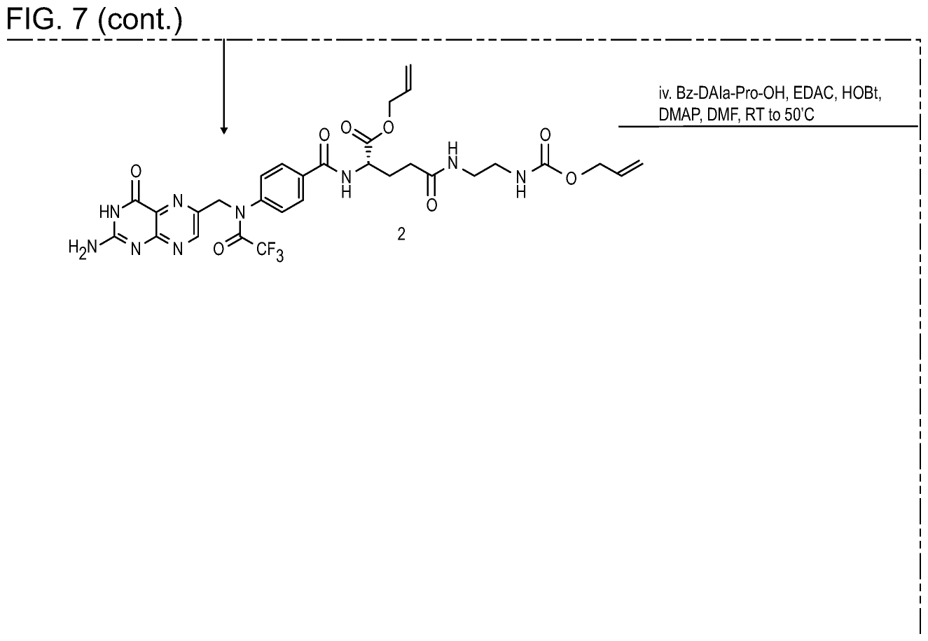

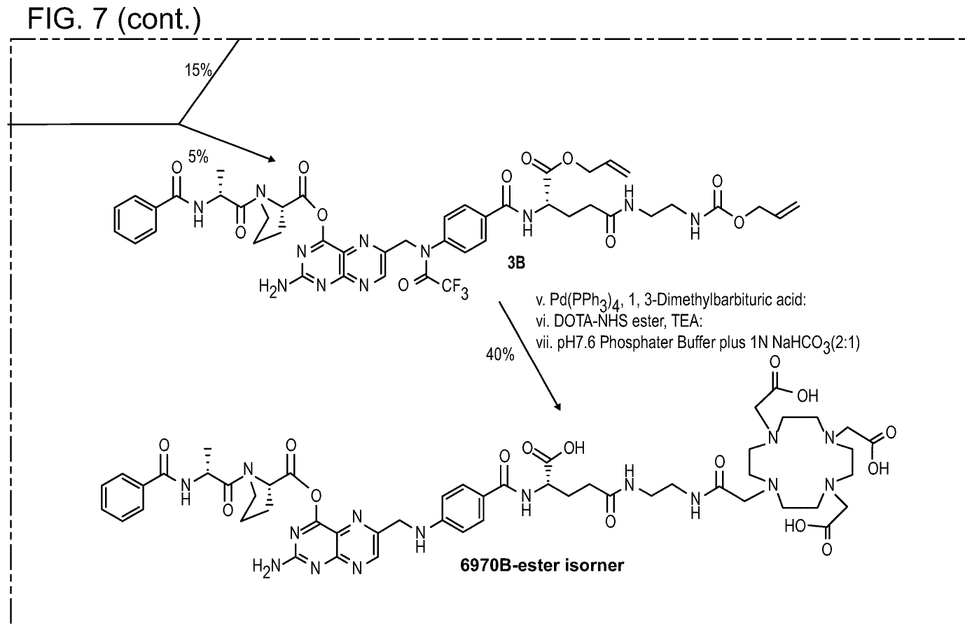

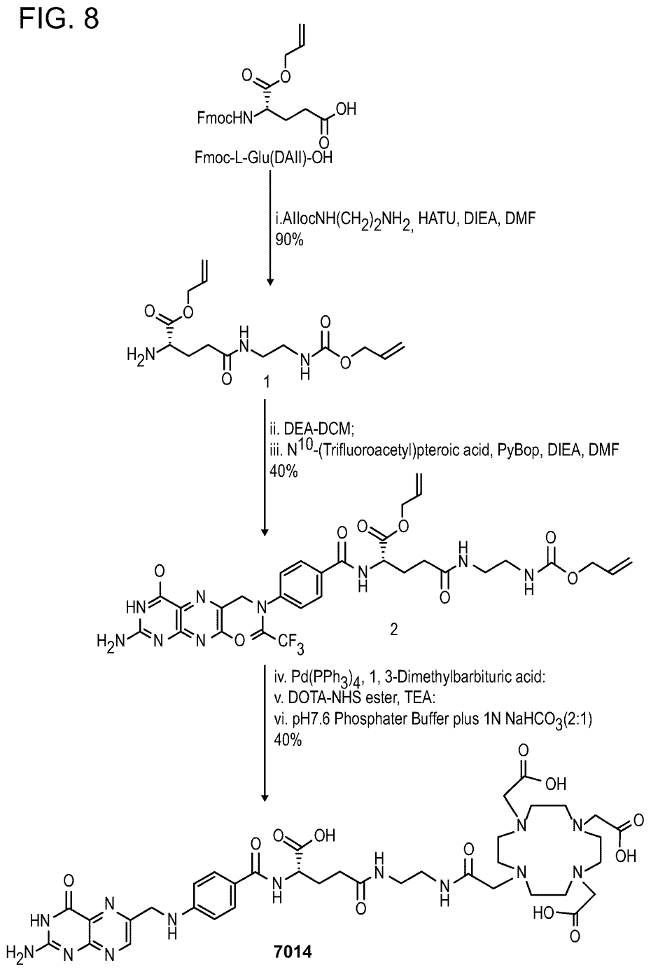

Results are depicted in FIG. 6.

### Example 8: Additional Syntheses and Compounds (FAP-Activated Folate/Methotrexate [MTX] Prodrug Complexes)

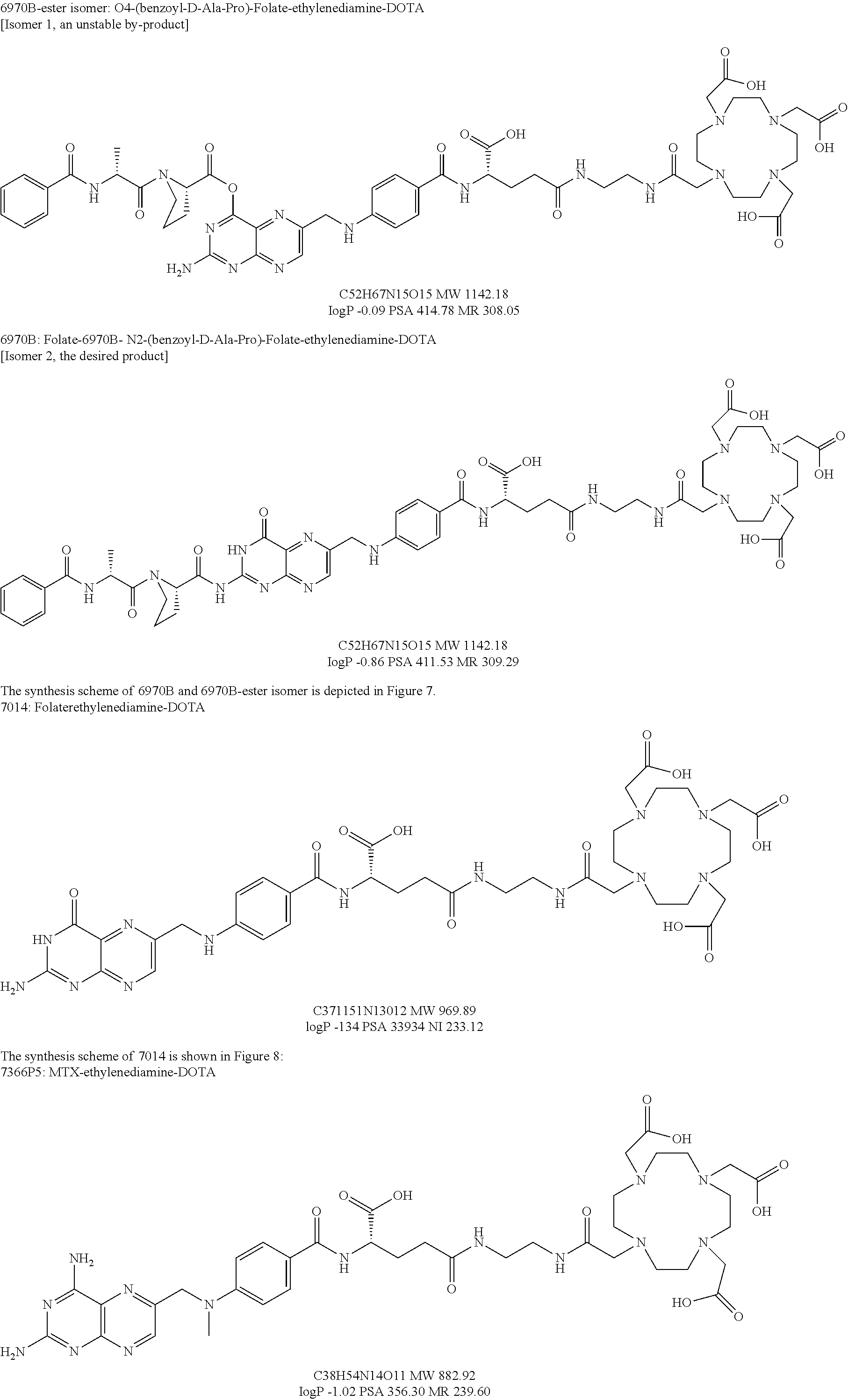

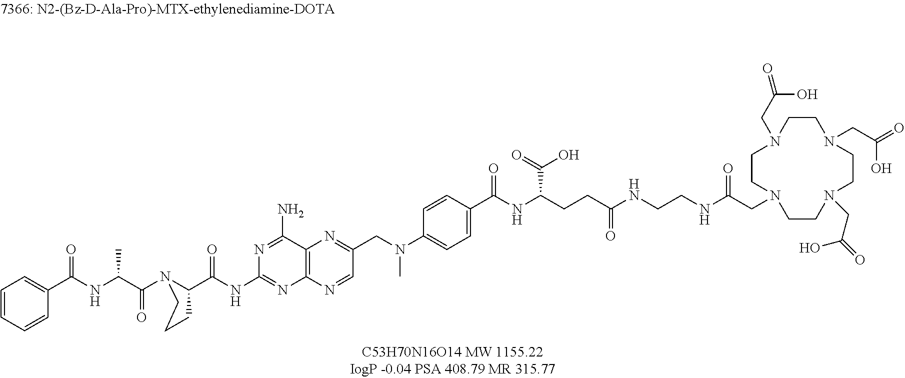

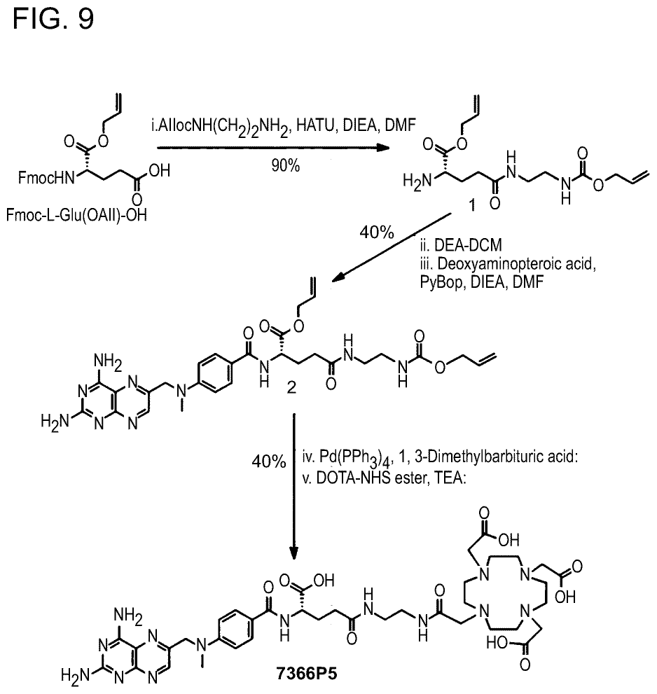

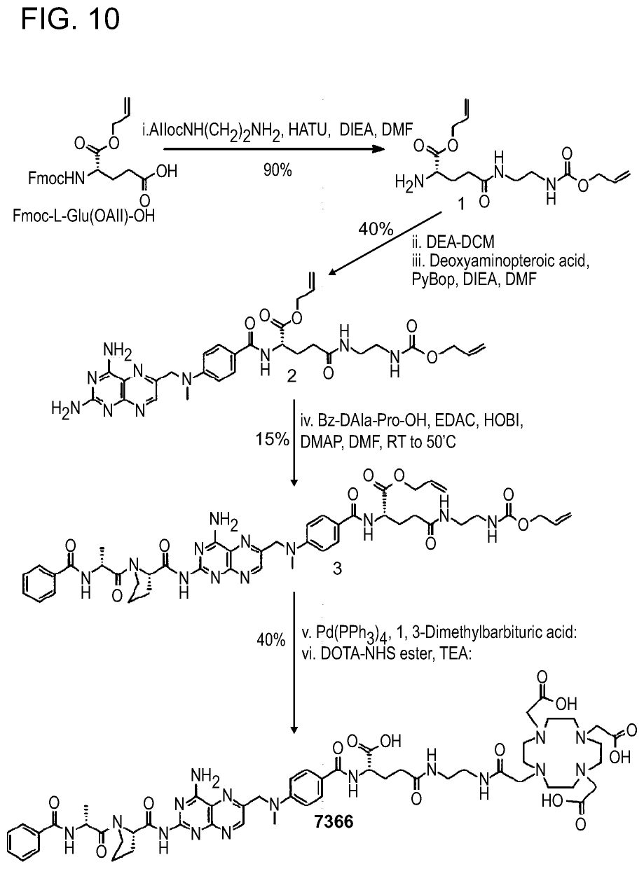

The synthesis scheme of 7366P5 is shown in FIG. 9, and 7366 is shown in FIG. 10.

### Example 9: FAP Activation of 6970B Isomer 1&2 and 7366

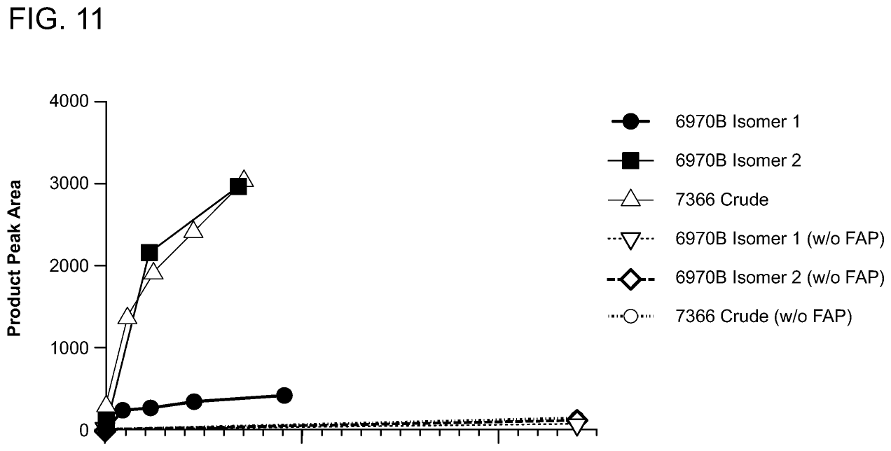

Results of FAP Activation of 6970B Isomer 1&2 and 7366100 uM Substrate, 50 nM FAP are shown in FIG. 11.

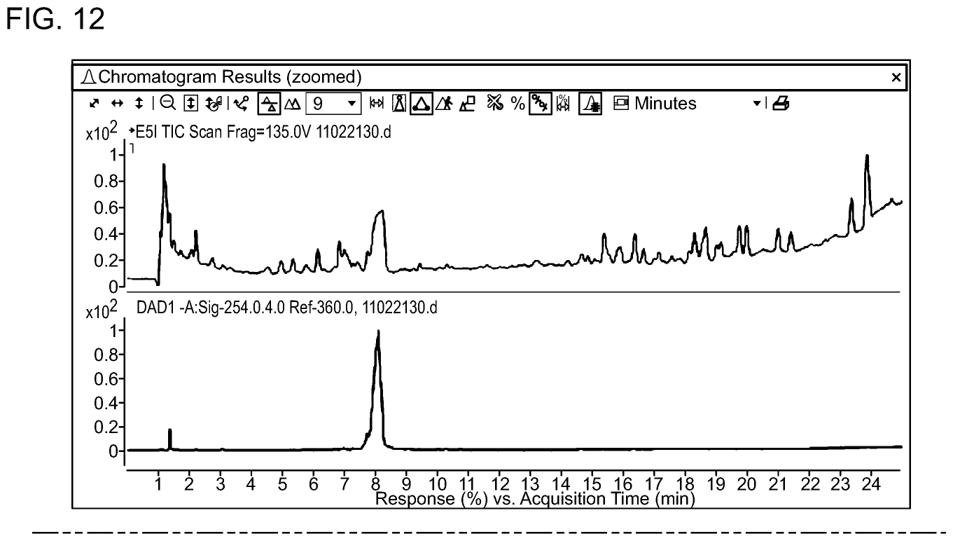

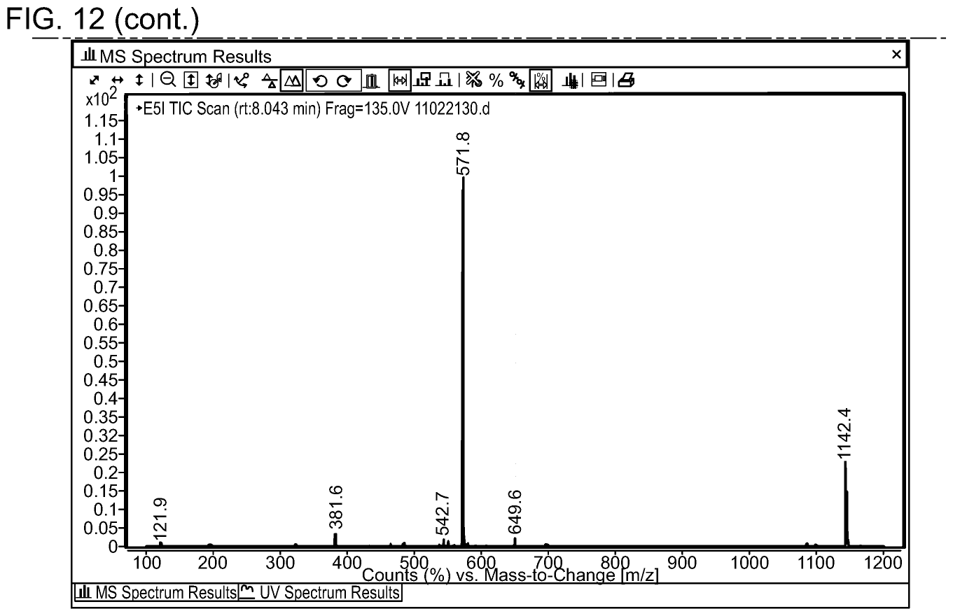

LC/MS spectra of 6970B Isomer 1@0.1 mM in FAP assay buffer is shown in FIG. 12.

### Instrumentation:

Agilent 1290 HPLC/6460 Triple Quad LC/MS
column: Zorbax Eclipse Plus C18, 4.6×50 mm 1.8 Um
HPLC method:
Mobile Phase A: 0.1% TFA in water
Mobile Phase B: 0.08% TFA in ACN
Flow rate: 0.5 mL/min
Gradient: 0-3 min, 10% B; 25 min, 98% B;

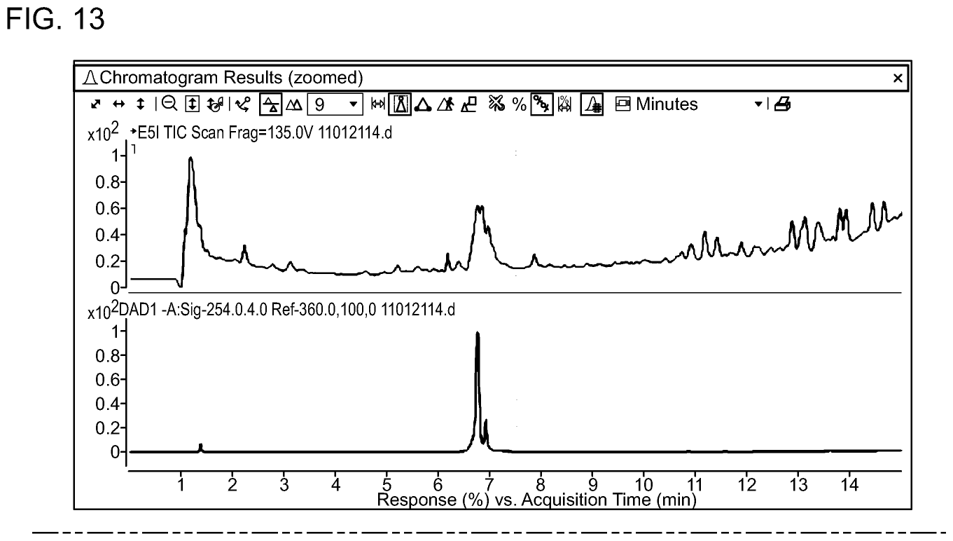

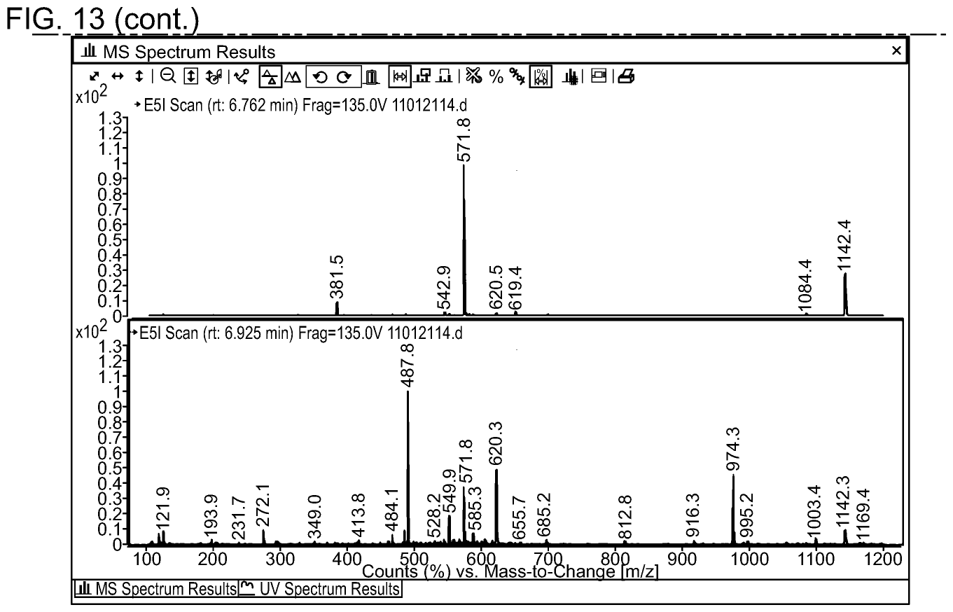

### LC/MS Spectra of 6970B Isomer 2 (0.1 mM in FAP Assay Buffer) is Shown in FIG. 13

### Instrumentation:

Agilent 1290 HPLC/6460 Triple Quad LC/MS
column: Zorbax Eclipse Plus C18, 4.6×50 mm 1.8 Um
HPLC method:
Mobile Phase A: 0.1% TFA in water
Mobile Phase B: 0.08% TFA in ACN
Flow rate: 0.5 mL/min
Gradient: 0-3 min, 10% B; 25 min, 98% B.

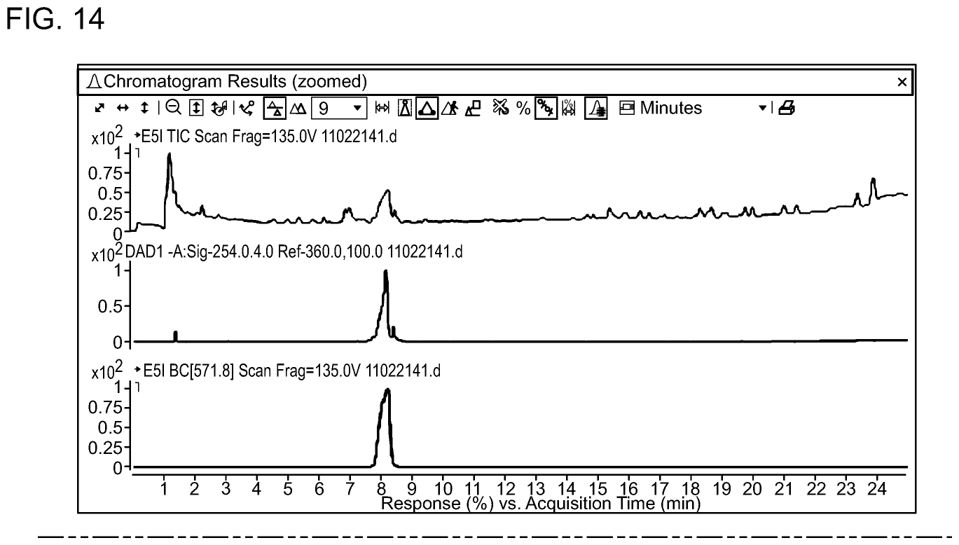

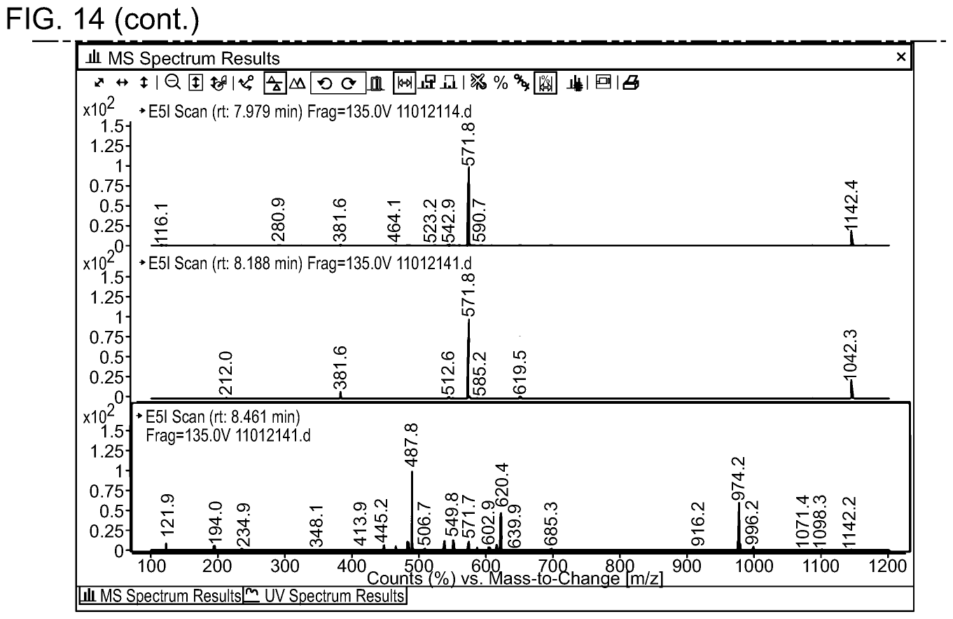

### LC/MS Spectra of 6970B Mixture Containing Isomer 1&2 is Shown in FIG. 14 Co-Inject 6970B Isomer 1 and Isomer 2

### Instrumentation:

Agilent 1290 HPLC/6460 Triple Quad LC/MS
column: Zorbax Eclipse Plus C18, 4.6×50 mm 1.8 Um
HPLC method:
Mobile Phase A: 0.1% TFA in water
Mobile Phase B: 0.08% TFA in ACN
Flow rate: 0.5 mL/min
Gradient: 0-3 min, 10% B; 25 min, 98% B.

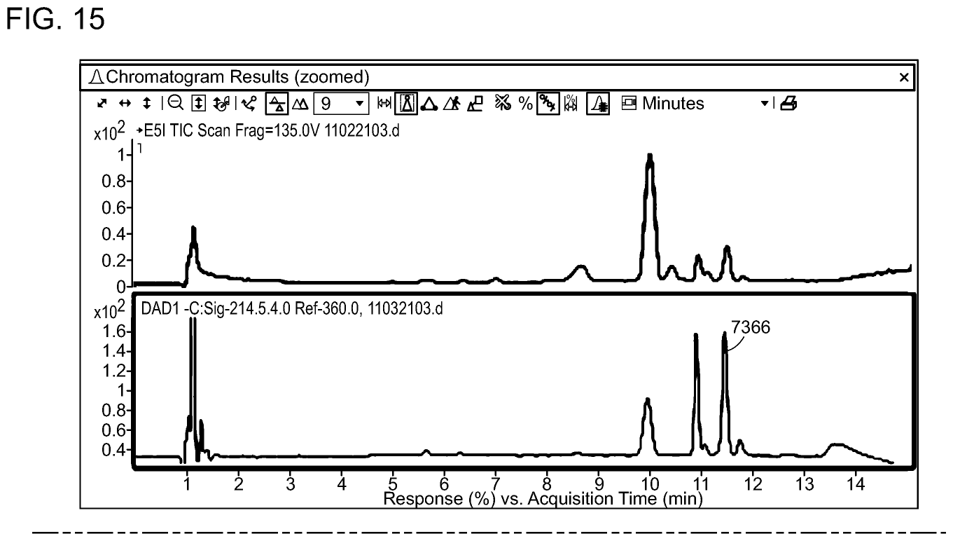

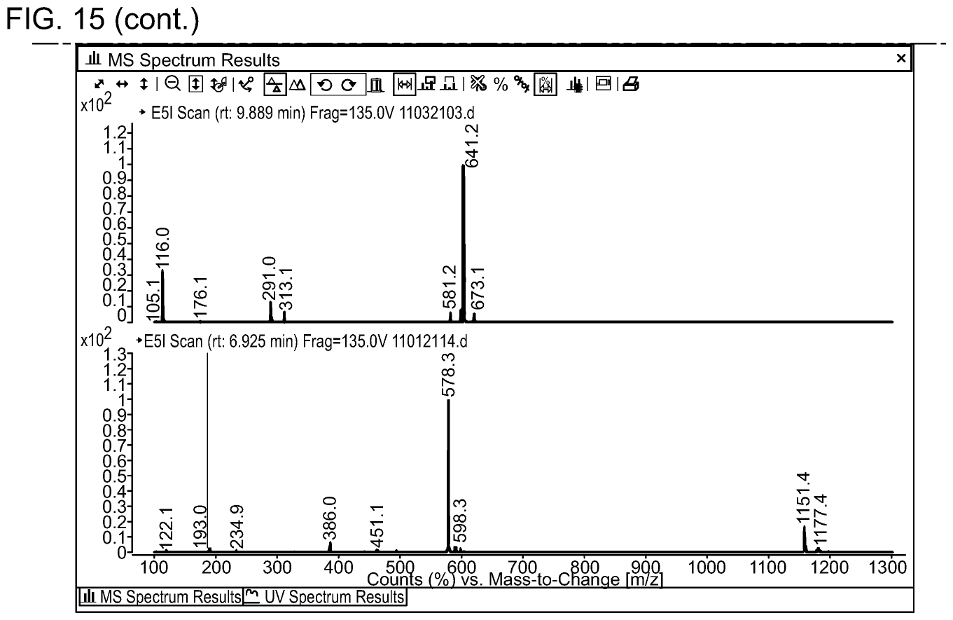

### LC/MS Spectra of 7366 is Shown in FIG. 15.

7366@0.1 mM in FAP buffer, T=0 min
Peak assignment:
Peak @12.6 min, MV.578.4/115.4, 7366, Ref.LC/MS (Peak 1)

### Instrumentation:

Agilent 1290 H PLC/6460 Triple Quad LC/MS
column: Zorbax Eclipse Plus C18, 4.6×50 mm 1.8 Um
H PLC method:
Mobile Phase A: 0.1% TFA in water
Mobile Phase B: 0.1% TFA in ACN
Flow rate: 0.5 mL/min
Gradient: 0-3 min, 10% B; 12 min, 26% B; 12-15 min, 98% B.

1-73. (canceled)

74. A fibroblast activation protein (FAP)-activated theranostic prodrug represented by the Formula II 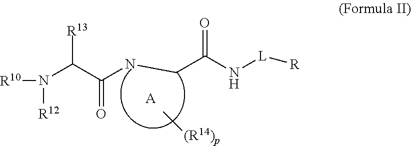
or a pharmaceutically acceptable salt thereof, wherein:
A represents a 5 to 8 membered heterocyclic ring;
R¹⁰ is R¹¹(C═X)—;
R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, —(C₆-C₁₄)aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl, wherein R¹¹ is optionally substituted with one or more substituents independently selected from the group consisting of halo, hydroxy, alkoxy, carboxy, cyano, amino, nitro, and thio; or R¹¹ is -(AA)ₙ-(C₁-C₁₀)alkyl, -(AA)ₙ-(C₁-C₁₀)alkoxy, -(AA)ₙ-(C₁-C₁₀)alkyl-C(O)—(C₁-C₁₀)alkyl, -(AA)ₙ-(C₃-C₈)cycloalkyl, -(AA)ₙ-(C₃-C₈)cycloalkyl(C₁-C₁₀)alkyl, -(AA)ₙ-(C₆-C₁₄)aryl, -(AA)ₙ-(C₆-C₁₄)aryl(C₁-C₁₀)alkyl, -(AA)ₙ-5-10-membered heteroaryl, or -(AA)ₙ-5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
R¹² is hydrogen or (C₁-C₆)alkyl;
R¹³ is hydrogen, or a (C₁-C₆)alkyl (which may be straight or branched chain);
R¹⁴ is, independently for each occurrence, —(C₁-C₆)alkyl, —OH, —NH₂, or halogen;
AA is, independently for each occurrence, an amino acid residue;
n is an integer from 1 to 5;
p is an integer from 0-6;
L is a bond, or after cleavage by FAP to release NH₂-L-R, is a self-eliminating linker;
R is represented by 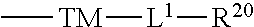
TM represents:
i) etarfolatide, vintafolide, leucovorin and methotrexate;
ii) octreotate, octreotide, pentetreotide, lanreotide, vapreotide, pasireotide, seglitide, benereotide, KE-108, SDZ-222-100, Sst3-ODN-8, CYN-154806, JR11, J2156, SRA-880, ACQ090, P829, SSTp-58, SSTp-86, BASS or somatoprim;
iii) cyclo(-Arg-Gly-Asp-D-Phe-Val-) [“c(RGDfV)” ], c(RGDfK), c(RGDfC), c(RADfC), c(RADfK), c(RGDfE), c(RADfE), RGDSK, RADSK, RGDS, c(RGDyC), c(RADyC), c(RGDyE), c(RGDyK), c(RADyK), H-E[c(RGDyK)]₂, EMD 12194, DMP728, DMP757 and SK&F107260;
L¹ represents a bond or a linker; and
R²⁰ represents a chelating agent.

75. The FAP-activated theranostic prodrug of claim 74, which is represented by Formula III 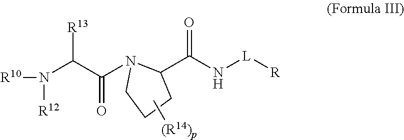
or a pharmaceutically acceptable salt thereof.

76. The FAP-activated theranostic prodrug of claim 75, which is represented by Formula IV 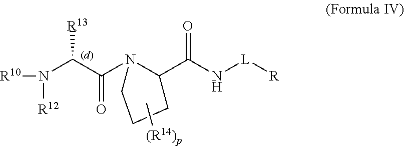
or a pharmaceutically acceptable salt thereof.

77. The FAP-activated theranostic prodrug of claim 74, wherein
(i) R¹³ is methyl;
(ii) R¹² is H; and/or
(iii) p is 0.

78. The FAP-activated theranostic prodrug of claim 75, which is represented by Formula VI: 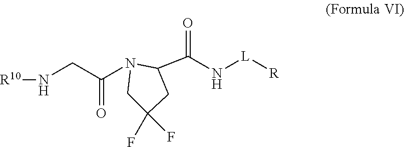
or a pharmaceutically acceptable salt thereof.

79. A fibroblast activation protein (FAP)-activated theranostic prodrug, represented by Formula VII: 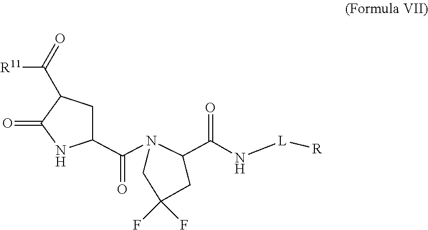
or a pharmaceutically acceptable salt thereof, wherein:
R is a ligand-targeted theranostic moiety, including a ligand for binding to a cellular target and a chelating agent for chelating a radioactive moiety;
R¹¹ is —(C₁-C₁₀)alkyl, —(C₁-C₁₀)alkoxy, —(C₁-C₁₀)alkyl-C(O)—OH, —(C₁-C₁₀)alkenyl-C(O)—OH, —(C₁-C₁₀)alkyl-C(O)—(C₁-C₁₀)alkyl, —(C₃-C₈)cycloalkyl, —(C₃-C₈)cycloalkyl(C₁-C₁₀)alkyl, —(C₆-C₁₄)aryl, —(C₆-C₁₄)aryl(C₁-C₁₀)alkyl, —O—(C₁-C₄)alkyl-(C₆-C₁₄)aryl, 5-10-membered heteroaryl, or 5-10-membered heteroaryl(C₁-C₁₀)alkyl, wherein R¹¹ is optionally substituted with one or more substituents independently selected from the group consisting of halo, hydroxy, alkoxy, carboxy, cyano, amino, nitro, and thio; and
L is a bond, or after cleavage by FAP to release NH₂-L-R, is a self-eliminating linker.

80. The FAP-activated theranostic prodrug of claim 79, wherein X is O.

81. The FAP-activated theranostic prodrug of claim 79, wherein R¹¹ is —(C₁-C₁₀)alkyl, —(C₁-C₁₀)alkoxy, —(C₃-C₈)cycloalkyl, —(C₆-C₁₄)aryl, —(C₆-C₁₄)aryl(C₁-C₁₀)alkyl, or 5-10-membered heteroaryl.

82. The FAP-activated theranostic prodrug of claim 81, wherein R¹¹ is

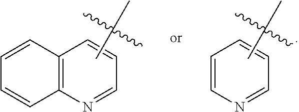

83. The FAP-activated theranostic prodrug of claim 79, wherein n equals 1, and AA is a serine residue.

84. The FAP-activated theranostic prodrug of claim 79, wherein n is 1 or 2.

85. The FAP-activated theranostic prodrug of claim 79, wherein R¹¹ is (C₁-C₁₀)alkyl, (C₁-C₁₀)alkoxy, (C₁-C₁₀)alkyl-C(O)—(C₁-C₁₀)alkyl, (C₃-C₈)cycloalkyl, (C₃-C₈)cycloalkyl(C₁-C₁₀)alkyl, (C₆-C₁₄)aryl, (C₆-C₁₄)aryl(C₁-C₁₀)alkyl, 5-10-membered heteroaryl, or 5-10-membered heteroaryl(C₁-C₁₀)alkyl, wherein R¹¹ is optionally substituted with one or more substituents independently selected from the group consisting of halo, hydroxy, carboxy, cyano, amino, nitro, and thio;
R¹² is hydrogen;
R¹³ is a (C₁-C₆)alkyl;
R¹⁴ is absent or p is 2 and R¹⁴ is a halogen for each occurrence; and
L is a bond, or —N(H)-L- is a self-eliminating linker.

86. The FAP-activated theranostic prodrug of claim 74, wherein —C(O)—R¹¹ forms an acyl of a carboxylic acid.

87. The FAP-activated theranostic prodrug of claim 86, wherein —C(O)—R¹¹ is formyl, acetyl, propionyl, butyryl, oxalyl, malonyl, succinyl, glutaryl, adipoyl, acryloyl, maleoyl, fumaroyl, glycoloyl, lactoyl, pyruvoyl, glyceroyl, maloyl, oxaloacetyl, benzoyl, trifluoroacetyl or methoxysuccinyl group.

88. The FAP-activated theranostic prodrug of claim 79, wherein R¹¹ is —(CH₂)ₘ—R^{11a}, where R^{11a} is a 5-10-membered aryl or heteroaryl group, and m is an integer from 1 to 6.

89. The FAP-activated theranostic prodrug of claim 88, wherein the aryl of R^{11a} is selected from the group consisting of benzyl, naphthalenyl, phenanthrenyl, phenolyl, and anilinyl, and the heteroaryl is selected from the group consisting of pyrryl, furyl, thiophenyl (a.k.a. thienyl), imidazolyl, oxazolyl, thiazolyl, triazolyl, pyrazolyl, pyridinyl, pyrazinyl, pyridazinyl, and pyrimidinyl.

90. The FAP-activated theranostic prodrug of claim 74, wherein
(i) L is a self-eliminating linker, wherein the self-eliminating linker is selected from the group consisting of 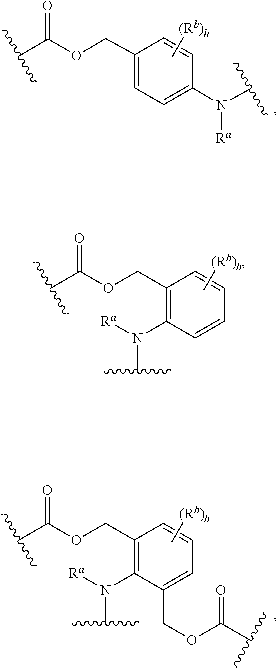 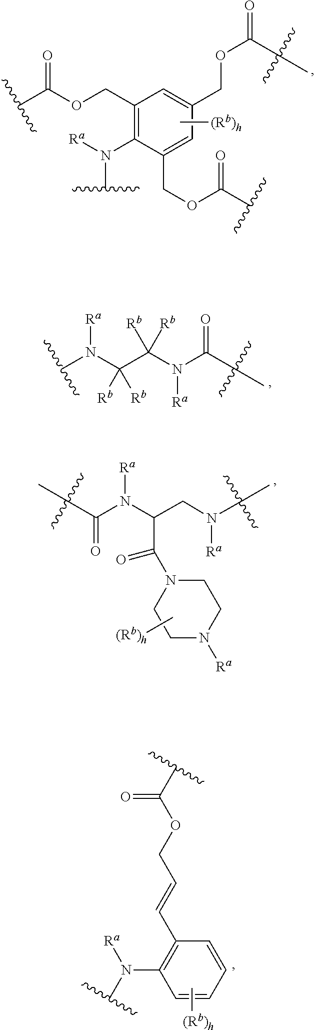 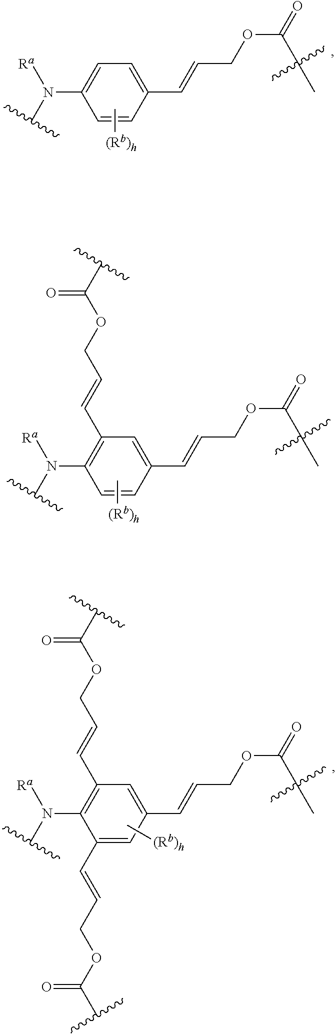 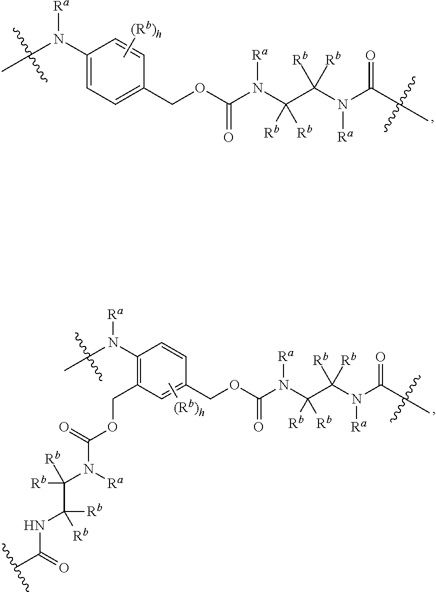 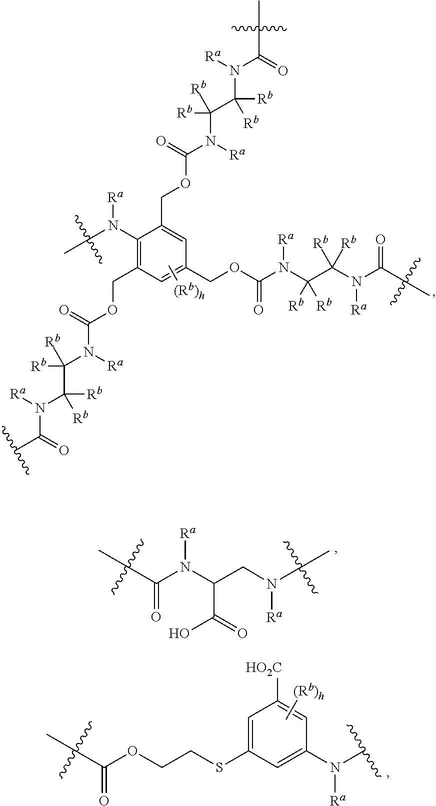 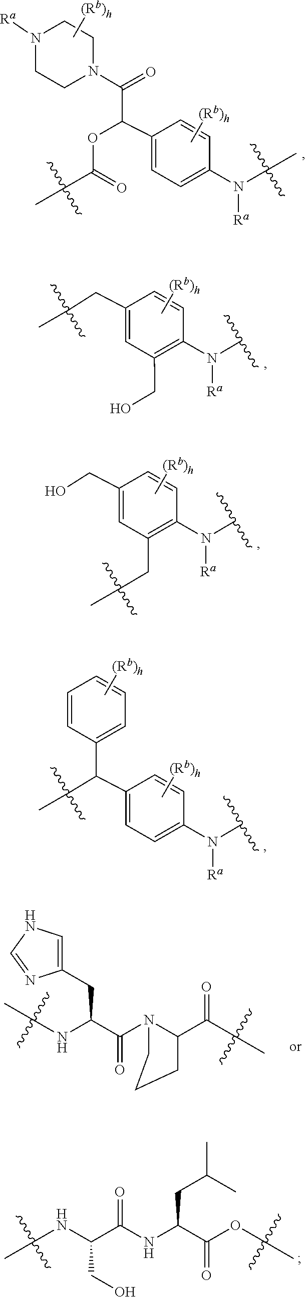
R^{a} is hydrogen, alkyl, alkenyl, alkynyl, carbocyclyl, heterocyclyl, aryl, or heteroaryl;
R^{b} is halogen, alkyl, alkenyl, alkynyl, carbocyclyl, heterocyclyl, aryl, or heteroaryl;
h is an integer from 0-8, as valency permits; and
i is an integer from 1-6; or
(ii) L is 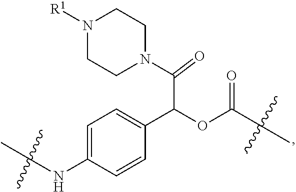
wherein R¹ is hydrogen, unsubstituted or substituted C₁₋₃ alkyl, or unsubstituted or substituted heterocyclyl; or
(iii) L is selected from 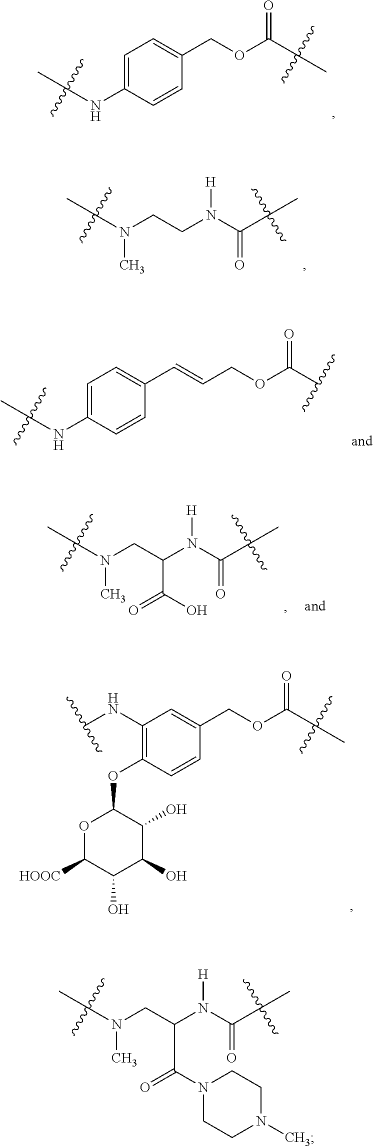
or
(iv) L is selected from 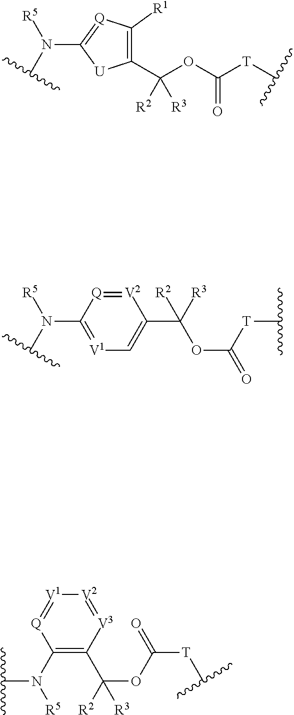
wherein
U is O, S or NR⁶;
Q is CR⁴ or N;
V¹, V² and V³ are independently CR⁴ or N provided that for formula II and III at least one of Q, V¹ and V² is N;
T is NH, NR⁶, O or S;
R¹, R², R³ and R⁴ are independently selected from H, F, Cl, Br, I, OH, —N(R⁵)₂, —N(R⁵)₃⁺, C₁-C₈ alkylhalide, carboxylate, sulfate, sulfamate, sulfonate, —SO₂R⁵, —S(═O)R⁵, —SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ halosubstituted alkyl, polyethyleneoxy, phosphonate, phosphate, C₁-C₈ alkyl, C₁-C₈ substituted alkyl, C₂-C₈ alkenyl, C₂-C₈ substituted alkenyl, C₂-C₈ alkynyl, C₂-C₈ substituted alkynyl, C₆-C₂₀aryl, C₆-C₂₀ substituted aryl, C₁-C₂₀ heterocycle, and C₁-C₂₀ substituted heterocycle; or when taken together, R² and R³ form a carbonyl (═O), or spiro carbocyclic ring of 3 to 7 carbon atoms; and
R⁵ and R⁶ are independently selected from H, C₁-C₈ alkyl, C1-C₈substituted alkyl, C₂-C₈ alkenyl, C₂-C₈ substituted alkenyl, C₂-C₈alkynyl, C₂-C₈ substituted alkynyl, C₆-C₂₀aryl, C₆-C₂₀ substituted aryl, C₁-C₂₀ heterocycle, and C₁-C₂₀ substituted heterocycle; where C₁-C₈ substituted alkyl, C₂-C₈ substituted alkenyl, C₂-C₈ substituted alkynyl, C₆-C₂₀ substituted aryl, and C₂-C₂₀ substituted heterocycle are independently substituted with one or more substituents selected from F, Cl, Br, I, OH, —N(R⁵)₂, —N(R⁵)₃⁺, C₁-C₈ alkylhalide, carboxylate, sulfate, sulfamate, sulfonate C₁-C₈ alkylsulfonate, C₁-C₈ alkylamino, 4-dialkylaminopyridinium, C₁-C₈ alkylhydroxyl, C₁-C₈ alkylthiol, —SO₂R⁵, —S(═O)R⁵, SR⁵, —SO₂N(R⁵)₂, —C(═O)R⁵, —CO₂R⁵, —C(═O)N(R⁵)₂, —CN, —N₃, —NO₂, C₁-C₈ alkoxy, C₁-C₈ trifluoroalkyl, C₁-C₈ alkyl, C₃-C₁₂ carbocycle, C₆-C₂₀ aryl, C₂-C₂₀ heterocycle, polyethyleneoxy, phosphonate, and phosphate; or
(v) L is selected from —NH—(C₂)₄—C(═O)— or —NH—(CH₂)₃—C(═O)—, p-aminobenzyloxycarbonyl (PABC), 2,4-bis(hydroxymethyl)aniline, or benzyloxycarbonyl.

91. The FAP-activated theranostic prodrug of claim 74, wherein the ligand-targeted theranostic moiety (R) is 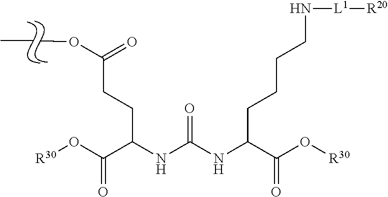
wherein
R³⁰ represents, independently for each occurrence, a hydrogen or a lower alkyl.

92. The FAP-activated theranostic prodrug of claim 74, wherein -L¹-R²⁰ is represented by 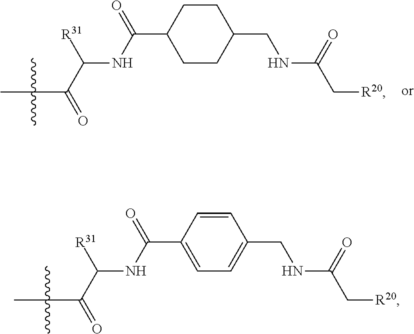
wherein R³¹ is —(CH₂)_{p′}-aryl or is —(CH₂)_{p′}-heteroaryl, and p′ is 0, 1, 2, 3 or 4.

93. The FAP-activated theranostic prodrug of claim 92, wherein R³¹ is —CH₂-aryl, where the aryl group is a C₆ to C₁₂ aryl, and is a monocyclic or bicyclic fused ring.

94. The FAP-activated theranostic prodrug of claim 74, wherein -L¹-R²⁰ is represented by

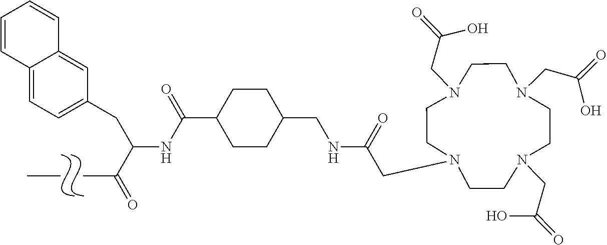

95. The FAP-activated theranostic prodrug of claim 79, wherein the ligand-targeted theranostic moiety (R) comprises 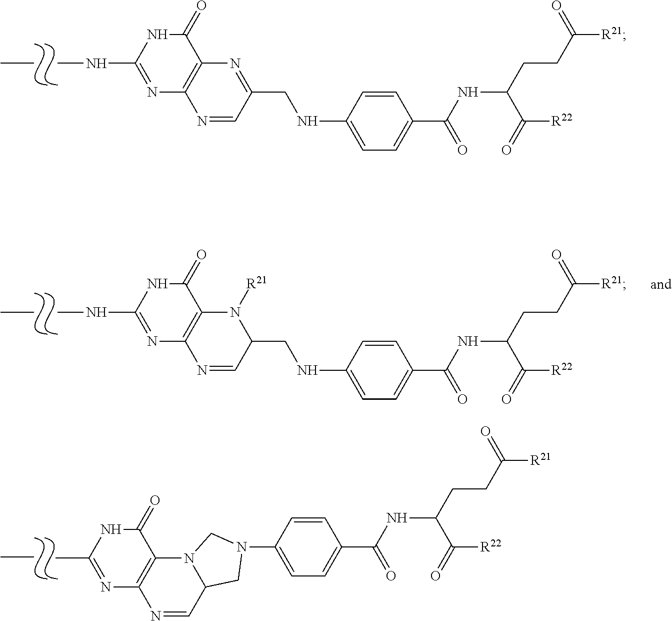
wherein
R²¹ represents H, and R²² represents —NH—(CH₂)_{q}—R²⁰, —NH—(CH₂)_{q}—NH—C(O)—(CH₂)_{q}—R²⁰ or —NH—(CH₂)_{q}—C(O)—(CH₂)_{q}—R²⁰; or
R²² represents H, and R²¹ represents —NH—(CH₂)_{q}—R²⁰ or —NH—(CH₂)_{q}—C(O)—(CH₂)_{q}—R²⁰; or
one of R²¹ or R²² represents H, and the other is selected from the group 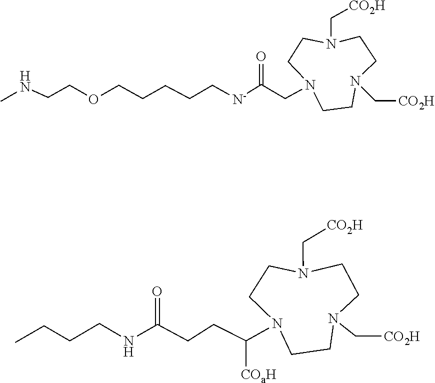
R²³ represents H, —CH₃, —CH₂CH₃, or —CO₂H;
and
q, independently for each occurrence, is 0, 1, 2, 3 or 4.

96. The FAP-activated theranostic prodrug of claim 95, wherein
(i) R²¹ represents —NH—CH₂—R²⁰, —NH—CH₂—C(O)—R²⁰, —NH—C(O)—CH₂—R²⁰, —NH—CH₂—C(O)—CH₂—R²⁰ or —NH—(CH₂)₂—NH—C(O)—CH₂—R²⁰ and R²² represents H; or
(ii) R²¹ represents H, and R²² represents —NH—CH₂—R²⁰, —NH—CH₂—C(O)—R²⁰, —NH—C(O)—CH₂—R²⁰, —NH—CH₂—C(O)—CH₂—R²⁰, or —NH—(CH₂)₂—NH—C(O)—CH₂—R²⁰.

97. The FAP-activated theranostic prodrug of claim 74, wherein the ligand-targeted theranostic moiety (R) is

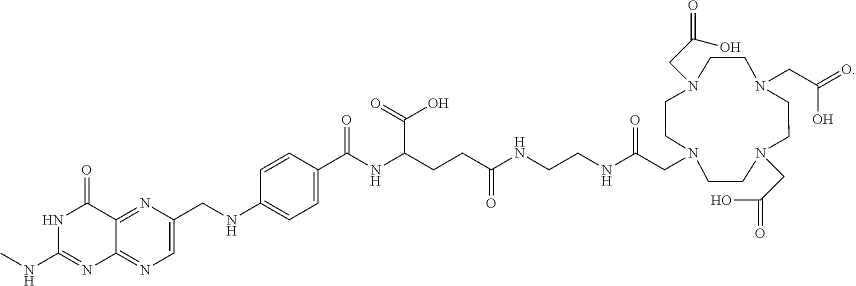 [SMILES: CNc1nc2ncc(CNc3ccc(C(=O)NC(CCC(=O)NCCNC(=O)CN4CCN(CC(=O)O)CCN(CC(=O)O)CCN(CC(=O)O)CC4)C(=O)O)cc3)nc2c(=O)[nH]1]

98. The FAP-activated theranostic prodrug of claim 74, wherein R is selected from the group consisting of:

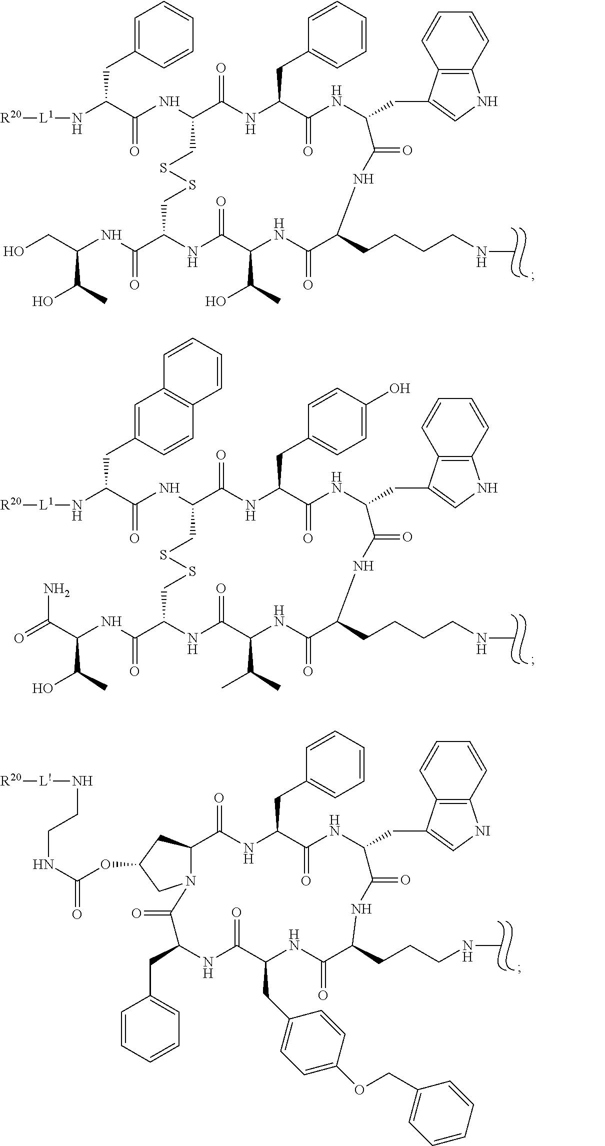

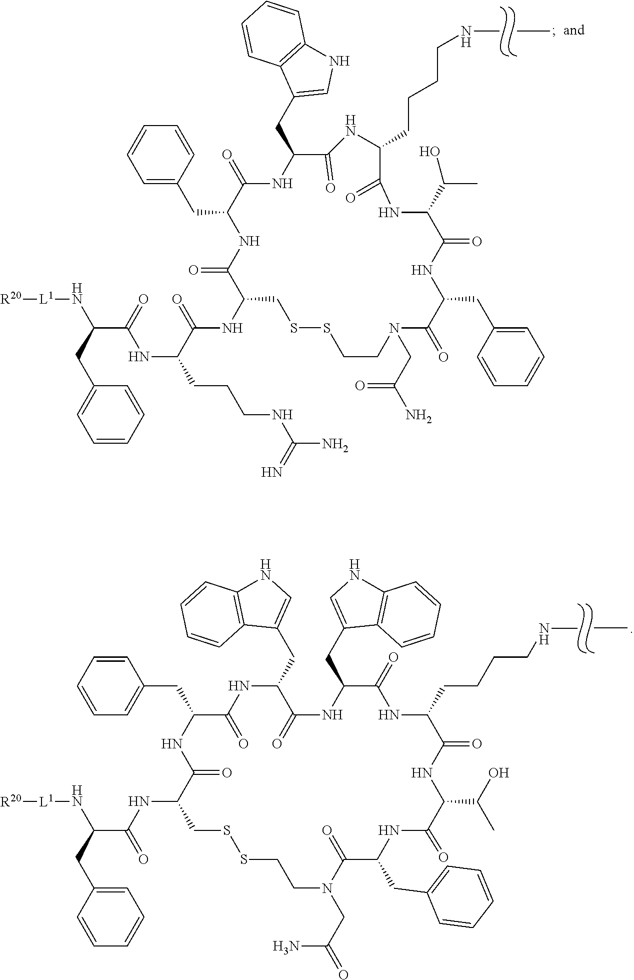

99. The FAP-activated theranostic prodrug of claim 74, wherein R is selected from the group consisting of:

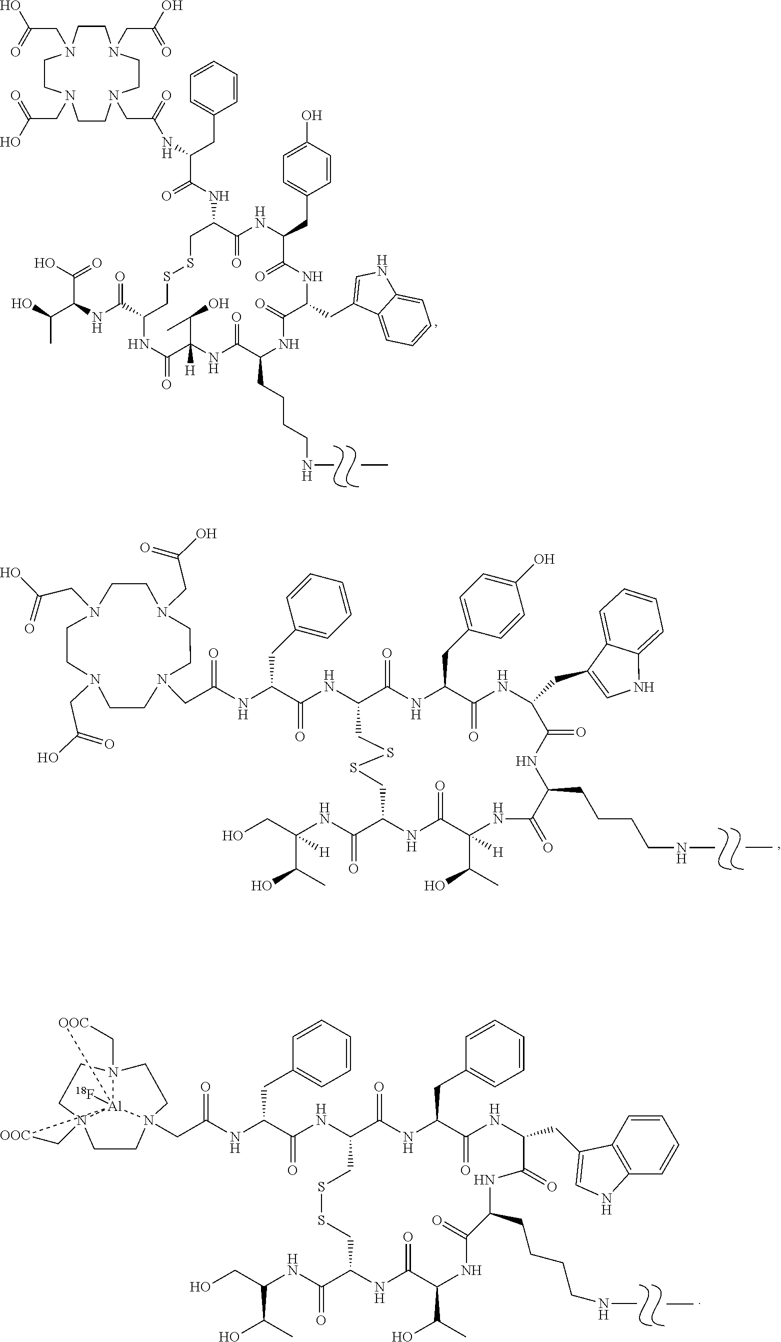

100. The FAP-activated theranostic prodrug of claim 74, having a structure selected from: 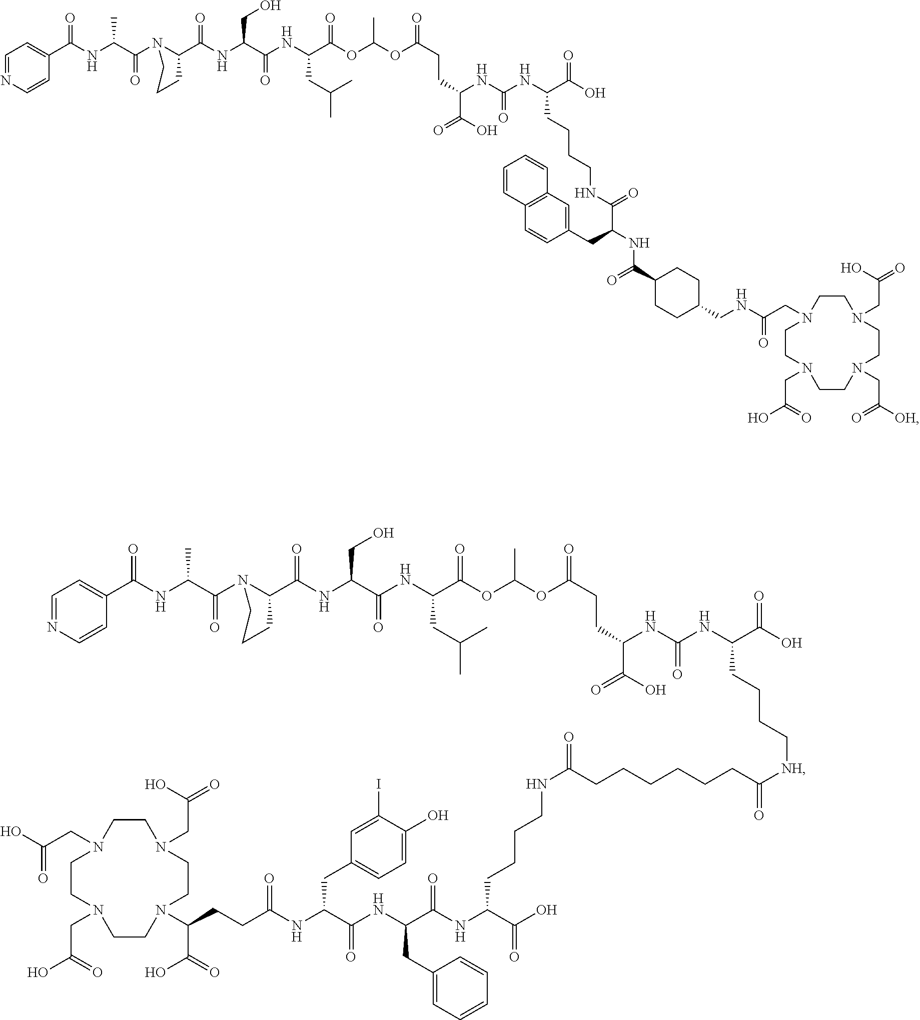 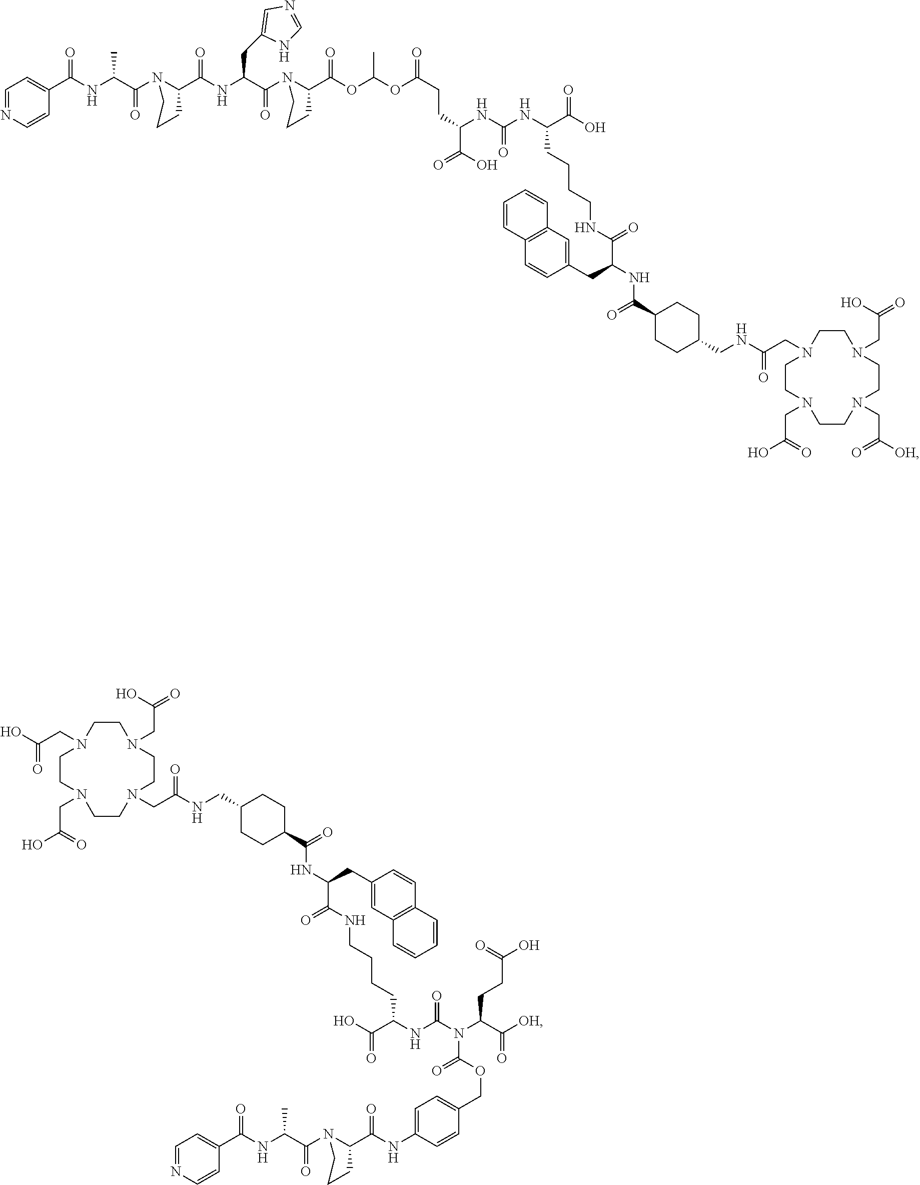 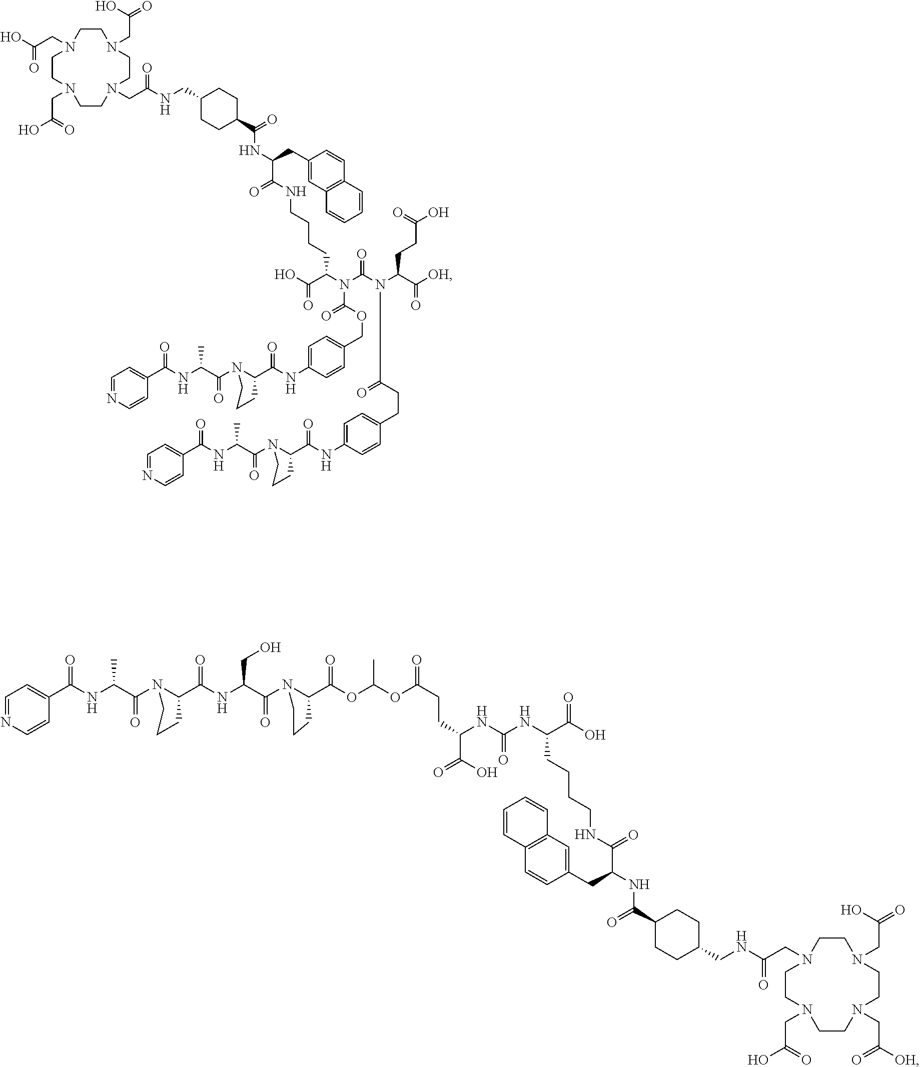 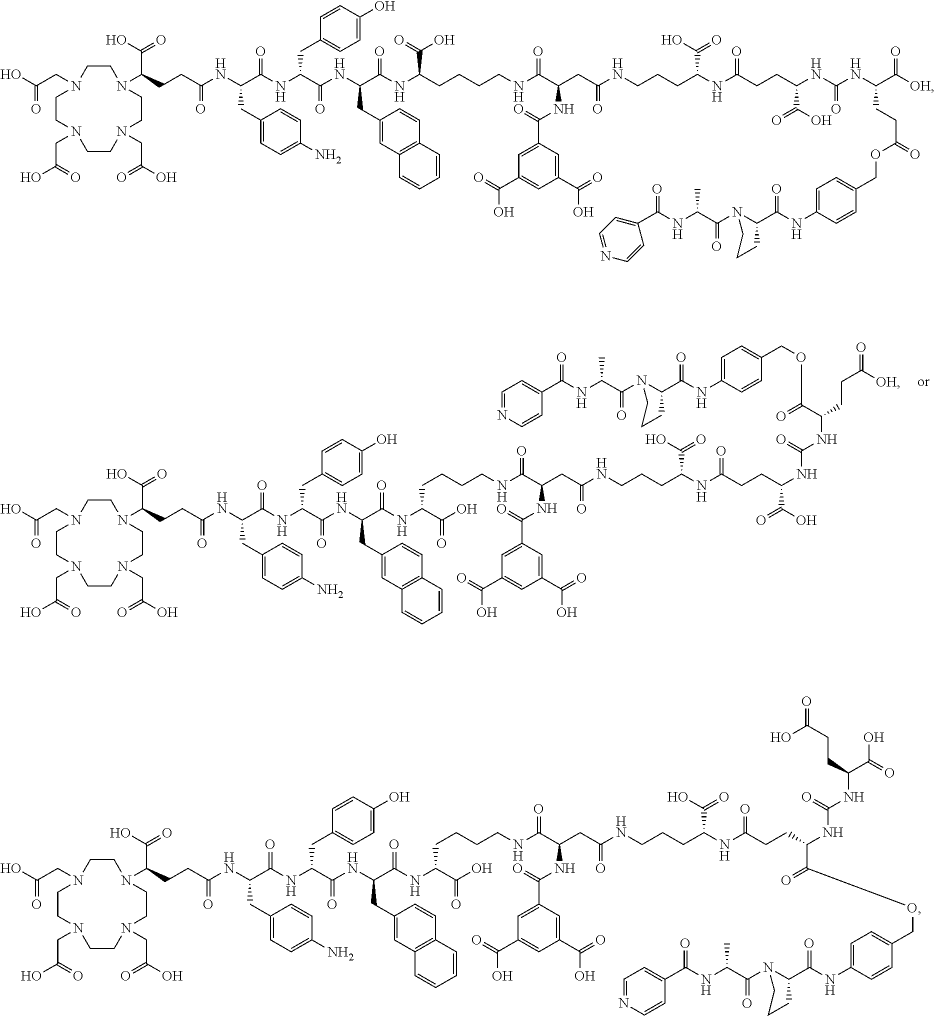
or a pharmaceutically acceptable salt thereof,
which may optionally include a radioisotope chelated thereto.

101. The FAP-activated theranostic prodrug of claim 74, having a structure selected from: 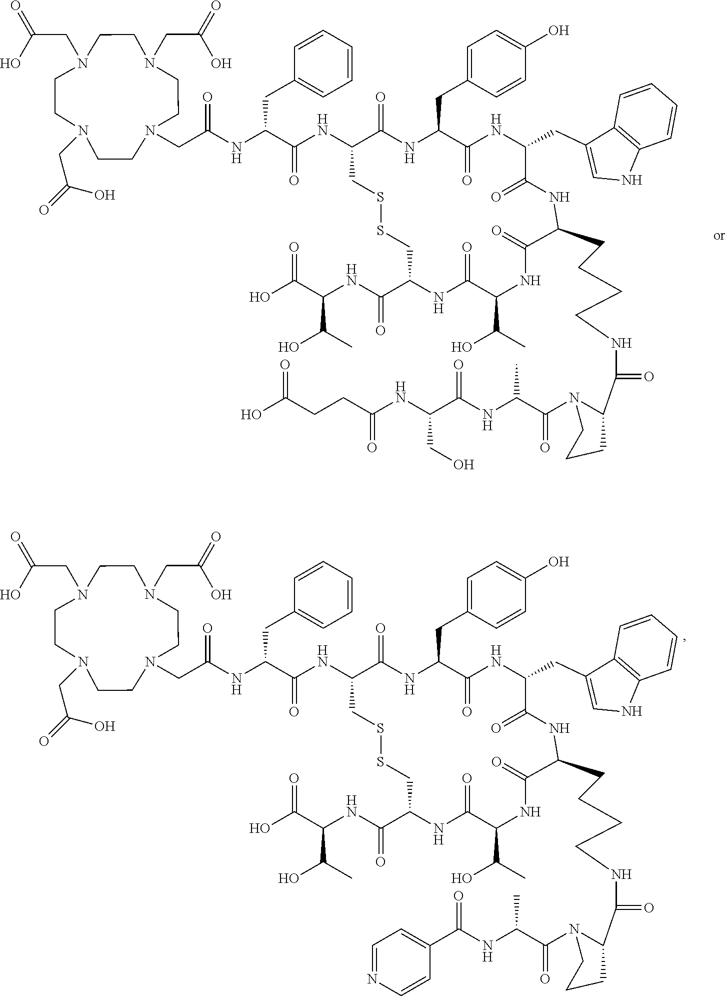
or a pharmaceutically acceptable salt thereof,
which may optionally include a radioisotope chelated thereto.

102. The FAP-activated theranostic prodrug of claim 74, wherein the prodrug comprises a radioactive isotope chelated thereto, wherein the radioactive isotope is selected from the group consisting of ¹⁸F, ⁴³K, ⁴⁷Sc, ⁵¹Cr, ⁵⁷Co, ⁵⁸Co, ⁵⁹Fe, ⁶⁴Cu, ⁶⁷Cu, ⁶⁷Ga, ⁶⁸Ga, ⁷¹Ge, ⁷²As, ⁷²Se, ⁷⁵Br, ⁷⁶Br, ⁷⁷As, ⁷⁷Br, ⁸¹Rb, ⁸⁸Y, ⁹⁰Y, ⁹⁷Ru, ^{99m}Tc, ¹⁰⁰Pd, ^{101m}Rh, ¹⁰³Pb, ¹⁰⁵Rh, ¹⁰⁹Pd, ¹¹¹Ag, ¹¹¹In, ¹¹³In, ¹¹⁹Sb, ¹²¹Sn, ¹²³I, ¹²⁴I, ¹²⁵I, ¹²⁷Cs, ¹²⁸Ba, ¹²⁹Cs, ¹³¹Cs, ¹³¹I, ¹³⁹La, ¹⁴⁰La, ¹⁴²Pr, ¹⁴³Pr, ¹⁴⁹Pm, ¹⁵¹Eu, ¹⁵³Eu, ¹⁵³Sm, ¹⁵⁹Gd, ¹⁶¹Tb, ¹⁶⁵Dy, ¹⁶⁶Ho, ¹⁶⁹Eu, ¹⁷⁵Yb, ¹⁷⁷Lu, ¹⁸⁶Re, ¹⁸⁸Re, ¹⁸⁹Re, ¹⁹¹Os, ¹⁹³Pt, ¹⁹⁴Ir, ¹⁹⁷Hg, ¹⁹⁸Au, ¹⁹⁹Ag, ¹⁹⁹Au, ²⁰¹Tl, ²⁰³Pb, ²¹¹At, ²¹²Bi, ²¹²Pb, ²¹³Bi, ²²⁵Ac and ²²⁷Th.

103. The FAP-activated theranostic prodrug of claim 74, wherein the prodrug has a k_{cat}/Kₘ for cleavage by FAP at least 10-fold greater than for cleavage by prolyl endopeptidase.

104. A pharmaceutical composition, comprising an FAP-activated theranostic prodrug of claim 74, or a pharmaceutically acceptable salt thereof, and a pharmaceutically acceptable carrier.

105. A method of treating a disorder characterized by fibroblast activation protein (FAP) upregulation, comprising administering to a subject in need thereof a therapeutically effective amount of the FAP-activated theranostic prodrug of claim 74, or a pharmaceutically acceptable salt thereof.

106. The method of claim 105, wherein the disorder characterized by FAP upregulation is cancer.
